SEQUENCE LISTING (26 sequences)

SEQ ID NO: 1 - DNA, 1089 bp, Artificial Sequence
  Synthetic  Polynucleotide
gtcgacgccgccaccatgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttgataagaattc

SEQ ID NO: 2 - DNA, 1089 bp, Artificial Sequence
  Synthetic  Polynucleotide
gtcgacgccgccaccatgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctggctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctgataagaattc

SEQ ID NO: 3 - DNA, 1089 bp, Artificial Sequence
  Synthetic  Polynucleotide
gtcgacgccgccaccatgctgttcaacctgcgaatcctgctgaacaacgccgcttttcggaacgggcacaactttatggtccgcaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgccactgctgcagggcaaaagcctggggatgatcttcgaaaagcgcagtactcggaccagactgtcaaccgagactggattcgctctgctgggaggacacccttgttttctgacaactcaggacattcacctgggagtgaacgagtccctgaccgacactgctcgcgtcctgagctctatggccgacgctgtgctggctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtcaccatgaaaaccgctaaggtcgcagccagcgattggacctttctgcactgcctgcccagaaagcccgaagaggtggacgacgaggtcttctactctccacgctccctggtgtttcccgaagctgagaataggaagtggacaattatggcagtgatggtcagcctgctgactgattattcacctcagctccagaaaccaaagttctgataagaattc

SEQ ID NO: 4 - DNA, 1089 bp, Artificial Sequence
  Synthetic  Polynucleotide
gtcgacgccgccaccatgctgttcaacctgagaatcctgctgaacaacgccgcctttcggaacggccacaacttcatggtccgcaacttccgctgcggccagccactgcagaacaaggtgcagctgaagggcagagacctgctgaccctgaagaacttcaccggcgaggagatcaaatacatgctgtggctgagcgccgatctgaagttcagaatcaagcagaagggcgagtacctgccactgctgcagggcaaaagcctgggcatgatcttcgaaaagcgctccacccggaccagactgagcaccgagaccggcttcgctctgctgggaggccacccttgcttcctgacaacccaggacatccacctgggcgtgaacgagtccctgaccgacaccgccagagtgctgagctctatggccgacgccgtgctggctcgggtgtacaaacagtccgacctggataccctggccaaggaagcttccatcccaattattaacggcctgtccgacctgtatcaccccatccagattctggccgattacctgaccctgcaggagcactatagcagcctgaagggcctgacactgtcttggatcggcgacggaaataatatcctgcactctattatgatgtctgccgccaagtttggaatgcacctgcaggctgctacccctaaaggctacgaacccgatgcctctgtgacaaagctggctgaacagtacgccaaagagaacggcacaaagctgctgctgaccaacgaccctctggaggccgctcacggaggcaacgtgctgatcaccgatacctggatttctatgggacaggaggaagagaagaagaagcggctgcaggccttccagggctaccaggtcaccatgaaaaccgctaaggtggccgccagcgattggacctttctgcactgcctgcccagaaagcccgaagaggtggacgacgaggtgttctacagcccccggagcctggtgtttcccgaagctgagaatcggaaatggacaattatggctgtgatggtgtccctgctgactgattattctcctcaactgcagaaacctaaattttgataagaattc

SEQ ID NO: 5 - DNA, 1089 bp, Artificial Sequence
  Synthetic  Polynucleotide
gtcgacgccgccaccatgctgttcaacctgcgcatcctgctgaacaacgccgccttccgcaacggccacaacttcatggtcagaaacttccgctgcggccagcccctgcaaaacaaagtgcagctgaagggccgcgacctgctgaccctgaagaacttcaccggcgaggagatcaaatacatgctgtggctgagcgccgacctgaagttccgcatcaagcagaagggcgagtacctgccactgctgcaaggcaagagcctgggcatgatcttcgagaagcgcagcacccgcacccgcctgagcaccgagaccggcttcgccctgctgggcggccacccctgcttcctgacaacacaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaatatcctgcacagtattatgatgagcgccgccaagttcggaatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatctctatgggccaggaggaggagaagaagaagcgcctgcaggccttccagggctaccaggtcactatgaagaccgccaaggtggccgccagcgactggaccttcctgcactgcctgccccgcaagcccgaggaggtggacgacgaggtgttctacagcccccgcagcctggtgttccccgaggccgagaaccgcaagtggactattatggccgtgatggtctccctgctgaccgactacagcccccagctgcagaagcccaagttctgataagaattc

SEQ ID NO: 6 - DNA, 1089 bp, Artificial Sequence
  Synthetic  Polynucleotide
gtcgacgccgccaccatgctgttcaacctgcggatcctgctgaacaacgccgccttcagaaacggccacaacttcatggtccgaaacttcagatgcggccagcctctgcagaacaaggtgcagctgaagggcagagatctgctgaccctgaagaacttcaccggcgaagagatcaaatacatgctgtggctgagcgccgacctgaagttccggattaagcagaagggcgagtacctgccactgctgcagggaaagtctctgggcatgatcttcgagaagcggagcaccagaaccagactgagcaccgagacaggctttgccctgctcggaggacacccctgctttctgacaacacaggatatccacctgggcgtgaacgagagcctgaccgatacagccagagtgctgagcagcatggctgatgccgtgctggccagagtgtacaagcagagcgatctggacaccctggccaaagaggccagcattcccatcatcaacggcctgagcgacctgtatcaccccatccagatcctggccgactacctgacactgcaagagcactacagcagcctgaagggactgaccctgtcttggatcggcgacggcaacaacatcctgcactctattatgatgagcgccgccaagttcggaatgcacctgcaggccgctacacccaagggctatgagcctgatgccagcgtgacaaagctggccgagcagtacgccaaagagaacggcacaaagctgctgctgaccaacgatcccctggaagctgcccacggcggcaatgtgctgatcaccgatacctggatctctatgggccaagaggaagagaagaagaagcggctgcaggccttccagggctaccaagtgacaatgaagaccgccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtccctgctgaccgactactctccccagctgcagaagcccaagttctgataagaattc

SEQ ID NO: 7 - DNA, 1089 bp, Artificial Sequence
  Synthetic  Polynucleotide
gtcgacgccgccaccatgctgttcaacctgcggatcctgctgaacaacgccgccttccggaacggccacaacttcatggtccggaacttccgctgtggccagcccctgcagaacaaggtgcagctgaagggcagggacctgctgaccctgaagaacttcaccggcgaagagatcaaatacatgctgtggctgagcgccgacctgaagttccggatcaagcagaagggcgagtacctgccactgctgcagggcaagtctctgggcatgatcttcgagaagcggagcacccggacccggctgtctaccgagacaggatttgccctgctgggcggccacccttgctttctgacaacacaggatatccacctgggcgtgaacgagagcctgaccgacacagccagagtgctgagcagcatggccgacgccgtgctggccagagtgtacaagcagagcgacctggacaccctggccaaagaggccagcatccccatcatcaacggcctgtccgacctgtaccaccccatccagatcctggcagactacctcacactgcaggaacactacagctccctgaagggcctgacactgagctggatcggcgacggcaacaatatcctgcactctattatgatgagcgccgccaagttcggaatgcacctgcaggccgccacccccaagggctacgagcctgacgccagcgtgaccaagctggccgagcagtacgccaaagagaacggcaccaagctgctgctgaccaacgaccctctggaagccgcccacggcggcaacgtgctgatcaccgatacctggatctctatgggccaggaagaggaaaagaagaagcggctgcaggccttccagggctaccaagtgacaatgaagaccgccaaagtggccgccagcgactggaccttcctgcactgcctgcccagaaagcccgaagaggtggacgacgaggtgttctacagcccccggtccctggtgtttcccgaggccgagaaccggaagtggacaattatggctgtgatggtgtctctgctgaccgactactccccccagctgcagaagcccaagttctgataagaattc

SEQ ID NO: 8 - DNA, 1086 bp, Artificial Sequence
  Synthetic  Polynucleotide
gtcgacgccgccaccatgctctttaatctgagaatcctgcttaacaacgccgccttccgcaacggacacaacttcatggtccggaacttcagatgcggccagcctctccaaaacaaggtccagcttaagggcagagatctgctcaccctcaaaaacttcaccggagaggaaatcaagtatatgctgtggctgtctgccgatcttaagttccggatcaaacagaagggggagtaccttccgctgctgcaagggaagtcactcggaatgatcttcgagaagcgctccactcggaccaggctcagcaccgaaactggatttgcactcctgggtggtcatccctgtttcctgaccacccaagatatccacctgggcgtgaacgaatccctgaccgacacagctcgcgtgctgtcctccatggccgacgctgtgttggcccgggtctacaagcagagcgacctggacactctggccaaggaagcctccattccgatcatcaatgggctgtccgacctgtaccacccaattcagatcctggcggattacttgaccctgcaagagcactacagctccctgaagggactgaccctctcctggattggcgacgggaacaacatcctccactcgattatgatgtcggcggcgaagttcggcatgcatctgcaagccgccactcctaagggttacgaaccggacgcaagcgtgaccaagctcgccgaacagtacgcgaaggaaaacggcactaagctgctgctgaccaacgaccccctggaagccgctcacggcggaaacgtgctgattacggacacctggatcagcatgggacaggaggaggagaagaagaagcggctgcaggcgttccagggataccaggtcaccatgaaaactgccaaagtggcagcctcagactggactttcctgcactgcctgcctcggaagccagaggaggtggacgatgaagtgttctactcccctcgctccctggtgttcccggaggcggaaaacaggaagtggaccatcatggccgtgatggtgtcattgttgaccgattactcgccgcaactgcagaagcccaagttttgagaattc

SEQ ID NO: 9 - DNA, 1089 bp, Artificial Sequence
  Synthetic  Polynucleotide
gtcgacgccgccaccatgctgtttaatctgagaatcctgctgaacaatgctgctttccgcaatgggcacaactttatggtccgaaacttccgatgtggacagcctctgcagaacaaggtgcagctgaagggccgggacctgctgaccctgaagaatttcacaggcgaggagatcaaatacatgctgtggctgagcgccgatctgaagtttaggatcaagcagaagggcgagtatctgccactgctgcagggcaagtccctgggcatgatcttcgagaagcggagcacccggacaagactgagcaccgagacaggattcgcactgctgggaggacacccatgctttctgacaacacaggacatccacctgggcgtgaacgagtctctgaccgacacagcacgggtgctgagctccatggcagatgccgtgctggccagagtgtacaagcagagcgacctggataccctggccaaggaggcctccatccccatcatcaatggcctgtctgacctgtatcacccaatccagatcctggccgattacctgaccctgcaggagcactattctagcctgaagggcctgacactgagctggatcggcgacggcaacaatatcctgcacagcatcatgatgtccgccgccaagtttggaatgcacctgcaggcagcaaccccaaagggatacgagcccgatgcctccgtgacaaagctggccgagcagtatgccaaggagaacggcaccaagctgctgctgacaaatgacccactggaggcagcacacggaggaaacgtgctgatcaccgatacatggatctctatgggccaggaggaggagaagaagaagcggctgcaggccttccagggctaccaggtgaccatgaagacagccaaggtggccgcctctgattggacctttctgcactgtctgccccggaagcctgaggaggtggacgatgaggtgttctattcccctcggagcctggtgttcccagaggcagagaatcgcaagtggacaatcatggccgtgatggtctccctgctgactgactactccccacagctgcagaagcccaagttttgataagaattc

SEQ ID NO: 10 - DNA, 1089 bp, Artificial Sequence
  Synthetic  Polynucleotide
gtcgacgccgccaccatgctcttcaatctccgaatactcctgaataatgcggcattcagaaatgggcacaattttatggtgcgaaattttcgatgtgggcagcccttgcaaaataaagtacaacttaaaggcagagatcttcttacgctcaaaaactttaccggcgaagagattaagtatatgttgtggctgtctgctgaccttaaattccgaataaaacagaaaggcgaataccttccgctcctccaagggaaatctcttgggatgattttcgagaagagatctacgcgcacgcggctttcaacggaaactggattcgcactcttgggcggccacccatgttttctgactactcaagatattcacttgggagtaaatgagtcacttactgacacggcaagagtgctctctagcatggctgatgcagtgctggctagagtctataaacaatccgacctggatacactcgccaaggaagcttcaataccaataatcaacgggttgtctgacttgtaccaccctatccaaatcttggccgattatttgacactccaggaacactactcaagtctgaagggacttacgcttagctggataggtgatggtaacaacatccttcatagcattatgatgtcagccgccaaattcggcatgcatctgcaagcagcgactcccaagggctatgagcctgatgcctcagtcaccaaactggcggagcagtacgctaaagagaatgggacgaagcttttgctgacgaacgatcccctggaggcggctcacgggggaaatgtgcttatcacggatacctggataagtatggggcaggaggaagaaaaaaaaaagcgattgcaagcctttcaaggttaccaggttacaatgaaaactgcgaaagtcgccgcatctgactggacttttctgcactgtcttccgagaaagccggaagaggtggacgacgaagtgttctactctccgcgctctctcgtgtttcctgaagcagagaaccgaaagtggaccataatggcggtaatggtcagcctcttgactgattattcccctcagctgcagaagccaaagttttgataagaattc

SEQ ID NO: 11 - DNA, 1089 bp, Artificial Sequence
  Synthetic  Polynucleotide
gtcgacgccgccaccatgctgttcaacctgcgcatcctgctgaacaacgccgccttccgcaacggccacaacttcatggtcagaaacttccgctgcggccagcccctgcaaaacaaagtgcagctgaagggccgcgacctgctgaccctgaagaacttcaccggcgaggagatcaaatacatgctgtggctgagcgccgatctgaagttcagaatcaagcagaagggcgagtacctgccactgctgcaaggcaagagcctgggcatgatcttcgagaagcgcagcacccgcacccgcctgagcaccgagaccggcttcgctctgctgggaggccacccttgcttcctgacaacccaggacatccacctgggcgtgaacgagagcctgaccgacaccgcccgcgtgctgagcagcatggccgacgccgtgctggctcgggtgtacaaacagtccgacctggataccctggccaaggaagcttccatccccatcatcaacggcctgagcgacctgtaccaccccatccagatcctggccgactacctgaccctgcaggagcactacagcagcctgaagggcctgaccctgagctggatcggcgacggcaacaatatcctgcactctattatgatgtctgccgccaagtttggaatgcacctgcaggctgctacccctaaaggctacgaacccgatgcctctgtgacaaagctggctgaacagtacgccaaagagaacggcacaaagctgctgctgaccaacgaccctctggaggccgctcacggaggcaacgtgctgatcaccgacacctggatctctatgggccaggaggaagagaagaagaagcggctgcaggccttccagggctaccaggtcaccatgaaaaccgctaaggtggccgccagcgattggaccttcctgcactgcctgccccgcaagcccgaggaggtggacgacgaggtgttctacagcccccgcagcctggtgttccccgaggccgagaaccgcaagtggactattatggccgtgatggtgtccctgctgactgattattctcctcaactgcagaaacctaaattttgataagaattc

SEQ ID NO: 12 - DNA, 1089 bp, Artificial Sequence
  Synthetic  Polynucleotide
gtcgacgccgccaccatgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaaatacatgctgtggctgagcgccgatctgaagttcagaatcaagcagaagggcgagtacctgcctctgctccagggcaaaagcctggggatgatcttcgaaaagcgcagtactcggaccagactgtcaaccgagactggcttcgctctgctgggaggccacccttgcttcctgacaacccaggacattcacctgggagtgaacgagtccctgaccgacactgctcgcgtcctgagctctatggccgacgccgtgctggctcgggtgtacaaacagtccgacctggataccctggccaaggaagcttccatccccatcatcaacggcctgagcgacctgtaccaccccatccagatcctggccgactacctgaccctgcaggagcactacagcagcctgaagggcctgaccctgagctggatcggcgacggcaacaatatcctgcactctattatgatgtctgccgccaagtttggaatgcacctgcaggctgctacccctaaaggctacgaacccgatgcctctgtgacaaagctggctgaacagtacgccaaagagaacggcacaaagctgctgctgaccaacgaccctctggaggccgctcacggaggcaacgtgctgatcaccgatacctggattagtatgggacaggaggaagagaagaagaagcggctgcaggccttccagggctaccaggtcaccatgaaaaccgctaaggtggccgccagcgattggacctttctgcactgcctgcccagaaagcccgaagaggtggacgacgaggtcttctactctcccagaagcctggtgtttcccgaagctgagaataggaagtggacaattatggcagtgatggtgtccctgctgactgattattctcctcaactgcagaaacctaaattttgataagaattc

SEQ ID NO: 13 - DNA, 1065 bp, Artificial Sequence
  Synthetic  Polynucleotide
atgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaaatacatgctgtggctgagcgccgatctgaagttcagaatcaagcagaagggcgagtacctgcctctgctccagggcaaaagcctggggatgatcttcgaaaagcgcagtactcggaccagactgtcaaccgagactggcttcgctctgctgggaggccacccttgcttcctgacaacccaggacattcacctgggagtgaacgagtccctgaccgacactgctcgcgtcctgagctctatggccgacgccgtgctggctcgggtgtacaaacagtccgacctggataccctggccaaggaagcttccatccccatcatcaacggcctgagcgacctgtaccaccccatccagatcctggccgactacctgaccctgcaggagcactacagcagcctgaagggcctgaccctgagctggatcggcgacggcaacaatatcctgcactctattatgatgtctgccgccaagtttggaatgcacctgcaggctgctacccctaaaggctacgaacccgatgcctctgtgacaaagctggctgaacagtacgccaaagagaacggcacaaagctgctgctgaccaacgaccctctggaggccgctcacggaggcaacgtgctgatcaccgatacctggattagtatgggacaggaggaagagaagaagaagcggctgcaggccttccagggctaccaggtcaccatgaaaaccgctaaggtggccgccagcgattggacctttctgcactgcctgcccagaaagcccgaagaggtggacgacgaggtcttctactctcccagaagcctggtgtttcccgaagctgagaataggaagtggacaattatggcagtgatggtgtccctgctgactgattattctcctcaactgcagaaacctaaattttga

SEQ ID NO: 14 - protein, 354 aa, Artificial Sequence
  Synthetic  Polypeptide
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 15 - protein, 354 aa, Artificial Sequence; identical to SEQ ID NO: 14
  Synthetic  Polypeptide

SEQ ID NO: 16 - protein, 354 aa, Artificial Sequence; identical to SEQ ID NO: 14
  Synthetic  Polypeptide

SEQ ID NO: 17 - protein, 354 aa, Artificial Sequence; identical to SEQ ID NO: 14
  Synthetic  Polypeptide

SEQ ID NO: 18 - protein, 354 aa, Artificial Sequence; identical to SEQ ID NO: 14
  Synthetic  Polypeptide

SEQ ID NO: 19 - protein, 354 aa, Artificial Sequence; identical to SEQ ID NO: 14
  Synthetic  Polypeptide

SEQ ID NO: 20 - protein, 354 aa, Artificial Sequence; identical to SEQ ID NO: 14
  Synthetic  Polypeptide

SEQ ID NO: 21 - protein, 354 aa, Artificial Sequence; identical to SEQ ID NO: 14
  Synthetic  Polypeptide

SEQ ID NO: 22 - protein, 354 aa, Artificial Sequence; identical to SEQ ID NO: 14
  Synthetic  Polypeptide

SEQ ID NO: 23 - protein, 354 aa, Artificial Sequence; identical to SEQ ID NO: 14
  Synthetic  Polypeptide

SEQ ID NO: 24 - protein, 354 aa, Artificial Sequence; identical to SEQ ID NO: 14
  Synthetic  Polypeptide

SEQ ID NO: 25 - protein, 354 aa, Artificial Sequence; identical to SEQ ID NO: 14
  Synthetic  Polypeptide

SEQ ID NO: 26 - protein, 354 aa, Artificial Sequence; identical to SEQ ID NO: 14
  Synthetic  Polypeptide

(19) United States
(12) Patent Application Publication
(10) Pub. No.: US 2020/0038462 A1
(43) Pub. Date: Feb. 6, 2020

(54) METHODS AND COMPOSITIONS OF OTC CONSTRUCTS AND VECTORS

(71) Applicant: Selecta Biosciences, Inc., Watertown, MA (US)

(72) Inventors: Peter Keller, Beverly, MA (US); Takashi Kei Kishimoto, Lexington, MA (US); Andres Muro, Watertown, MA (US); Giulia De Sabbata, Watertown, MA (US)

(73) Assignee: Selecta Biosciences, Inc., Watertown, MA (US)

(21) Appl. No.: 16/513,566

(22) Filed: Jul. 16, 2019

Related U.S. Application Data
(60) Provisional application No. 62/839,766, filed on Apr. 28, 2019.
(60) Provisional application No. 62/698,503, filed on Jul. 16, 2018.

(51) Int. Cl. A61K 35/76; C12N 15/86; A61K 47/69

(52) U.S. Cl. CPC A61K 35/76; B82Y 5/00; A61K 47/6937; C12N 15/86

(57) ABSTRACT

Provided herein are methods and compositions related to nucleic acids encoding ornithine transcarbamylase (OTC), such as nucleic acids comprising an OTC codon-optimized sequence, as well as related vectors, such as AAV vectors. Also, provided are methods for administering AAV vectors that comprise a sequence that encodes an enzyme associated with an urea cycle disorder and an expression control sequence, in combination with synthetic nanocarriers coupled to an immunosuppressant.

### RELATED APPLICATIONS

This application claims the benefit under 35 U.S.C. § 119(e) of U.S. Provisional Application Ser. No. 62/698,503, filed on Jul. 16, 2018 and U.S. Provisional Application Ser. No. 62/839,766, filed Apr. 28, 2019, the entire contents of each of which are incorporated herein by reference.

### FIELD OF THE INVENTION

The invention relates to methods and compositions related to nucleic acids encoding ornithine transcarbamylase (OTC), such as nucleic acids comprising an OTC codon-optimized sequence, as well as related vectors, such as AAV vectors. Also, provided are methods for administering AAV vectors that comprise a sequence that encodes an enzyme associated with an urea cycle disorder and an expression control sequence, in combination with synthetic nanocarriers coupled to an immunosuppressant.

### SUMMARY OF THE INVENTION

Provided herein are methods and compositions related to nucleic acids encoding OTC, such as nucleic acids comprising an OTC codon-optimized sequence, as well as related vectors, such as AAV vectors. Also, provided herein are methods and compositions for administering AAV vectors that comprise a nucleic acid sequence that encodes an enzyme associated with an urea cycle disorder and an expression control sequence, in combination with synthetic nanocarriers coupled to an immunosuppressant. The administration may have a therapeutic benefit for any one of the purposes provided herein in any one of the methods or compositions provided herein.

In another aspect a method or composition as described in any one of the Examples is provided. In an embodiment, a composition that comprises any one of the vectors or nucleic acid sequences provided herein is provided.

In another aspect, any one of the compositions is for use in any one of the methods provided.

In another aspect, any one of the methods or compositions is for use in treating any one of the diseases or disorders described herein. In another aspect, any one of the methods or compositions is for use in reducing an immune response (i.e., humoral and/or cellular) to an AAV antigen and/or the expressed product of the AAV vector, increasing expression of the sequence encoding the enzyme, or for repeated administration of an AAV vector.

### BRIEF DESCRIPTION OF THE FIGURES

FIG. 1 shows transfection efficiency of three different constructs. A GFP plasmid was used to normalize for transfection efficiency (wt).

FIG. 2 shows the results when each construct was transfected in duplicate. The Western blot (top) is quantified by band intensity in the graph (bottom) in WB quantification.

FIG. 3 shows the band intensity of each construct from all experiments (n=4).

FIG. 4 shows features of the CO3 sequence.

FIG. 5 shows features of the CO21 sequence.

FIG. 6 shows a variety of different algorithms used for codon optimization analysis, including codon usage, cryptic splicing sites, ORFs in the antisense strand (ARF >50 bp), secondary structure, GC-content, and CpG islands.

FIG. 7 shows OTC mRNA expression levels in Huh7 cells transfected with pSMD2_hOTC constructs using real-time PCR, n=2.

FIGS. 8A-8B show OTC expression in HUH7 transfected with pSMD2_hOTC constructs; a Western blot analysis (FIG. 8A) and band quantification (FIG. 8B) are shown.

FIG. 9 shows hOTC subcellular localization by staining.

FIG. 10 shows the results from AAV batch 5.0E12 vgp/kg in C57Bl/6N. Three different constructs were tested: AAV8-CO1, AAV8-CO3, and AAV8-CO6. AAV8-OTC wild-type was used as a control.

FIG. 11 shows the results of OTC^{spf-ash} mice (5×10¹¹ Vg/Kg) experiments.

FIG. 12 shows a comparison of human and mouse OTC by Western blot.

FIG. 13 shows the urinary orotic acid of OTC^{spf-ash} mice having the same (5×10¹¹ Vg/Kg) concentration of virus in liver (n=2).

FIG. 14 shows the expression levels of a first group of AAV8-hOTC-CO variants in HUH7 hepatocellular carcinoma lines. Six different constructs were tested: AAV8-hOTC-CO1, AAV8-hOTC-CO2, AAV8-hOTC-CO3, AAV8-hOTC-CO6, AAV8-hOTC-CO7, AAV8-hOTC-CO9. AAV8-hOTC wild-type and empty AAV8 vector were used as controls (n=2, *=P<0.05).

FIG. 15 shows the expression levels of a second group of AAV8-hOTC-CO variants in HUH7 hepatocellular carcinoma lines. Five different constructs were tested: AAV8-hOTC-CO1, AAV8-hOTC-CO3, AAV8-hOTC-CO6-1, AAV8-hOTC-CO9-1, AAV8-hOTC-CO9-2. AAV8-hOTC wild-type was used as a control (n=2, *=P<0.05).

FIG. 16 shows a logo representation of the alignment of 566 OTC sequences in humans. The numbering corresponds to the human sequence for removing insertions relative to the human sequence. The size of the letters indicates the degree of sequence conservation.

FIG. 17 shows a schematic representation of the shuffled hOTC cDNA constructs to generate a third group of hOTC-CO variants. The hOTC-CO21 and hOTC-CO18 constructs were designed by shuffling the conserved regions of the hOTC-CO1, hOTC-CO3, and hOTC-CO6 constructs. The numbering corresponds to the amino acid sequence of the wild-type human OTC protein.

FIG. 18 shows the expression levels of AAV8-hOTC-CO constructs in HUH7 hepatocellular carcinoma lines. Five different constructs were tested: AAV8-hOTC-CO1, AAV8-hOTC-CO3, AAV8-hOTC-CO6, AAV8-hOTC-CO18, and AAV8-hOTC-CO21. AAV8-hOTC wild-type was used as a control (n=4, *=P<0.05).

FIG. 19 shows expression levels of OTC, catalytic activity of OTC, and viral genome copies/cell in male C57Bl/6N mice transduced with high dose AAV (5.0E12 viral genomes/kilogram (vg/kg)). Six different constructs were tested: AAV8-hOTC-CO1, AAV8-hOTC-CO2, AAV8-hOTC-CO3, AAV8-hOTC-CO6, AAV8-hOTC-CO7, and AAV8-hOTC-CO9. AAV8-hOTC wild-type was used as a control (n=3, *=P<0.05).

FIG. 20 shows the results from male C57Bl/6N mice transduced with AAV (5.0E12 vg/kg). Three different constructs were tested: AAV8-hOTC-CO1, AAV8-hOTC-CO3, and AAV8-hOTC-CO6. AAV8-hOTC wild-type was used as a control (n=3, *=P<0.05).

FIG. 21 shows expression levels of OTC, catalytic activity of OTC, and viral genome copies/cell in male C57Bl/6N mice transduced with AAV (1.25E12 vg/kg). Six different constructs were tested: AAV8-hOTC-CO1, AAV8-hOTC-CO2, AAV8-hOTC-CO3, AAV8-hOTC-CO6, AAV8-hOTC-CO7, and AAV8-hOTC-CO9. AAV8-hOTC wild-type was used as a control (n=3, *=P<0.05, **=P<0.01, ***=P<0.001). Expression levels, catalytic activity of OTC, and viral genome copies/cell are shown.

FIG. 22 shows expression levels of OTC, catalytic activity of OTC, and viral genome copies/cell in female C57Bl/6N mice transduced with AAV (5.0E12 vg/kg). Six different constructs were tested: AAV8-hOTC-CO1, AAV8-hOTC-CO2, AAV8-hOTC-CO3, AAV8-hOTC-CO6, AAV8-hOTC-CO7, and AAV8-hOTC-CO9. AAV8-hOTC wild-type was used as a control.

FIG. 23 shows mRNA levels of AAV8-hOTC-CO constructs in male and female C57Bl/6N mice treated with 1.25E12 vg/kg or 5.0E12 vg/kg constructs. Six different constructs were tested: AAV8-hOTC-CO1, AAV8-hOTC-CO2, AAV8-hOTC-CO3, AAV8-hOTC-CO6, AAV8-hOTC-CO7, and AAV8-hOTC-CO9. AAV8-hOTC wild-type was used as a control.

FIG. 24 shows expression levels of OTC, catalytic activity of OTC, and viral genome copies/cell in male C57Bl/6N mice transduced with AAV (1.25E12 vg/kg). Three different constructs were tested: AAV8-hOTC-CO1, AAV8-hOTC-CO3, and AAV8-hOTC-006. AAV8-hOTC wild-type was used as a control (n=2, *=P<0.05).

FIG. 25 shows expression levels of OTC, catalytic activity of OTC, and viral genome copies/cell in C57Bl/6N mice transduced with AAV (1.25E12 vgp/kg). Three different constructs were tested: AAV8-hOTC-CO1, AAV8-hOTC-CO3, and AAV8-hOTC-CO21. AAV8-hOTC wild-type was used as a control (n=4, *=P<0.05).

FIG. 26 shows urinary orotic acid of OTC^{spf-ash} mice treated with 5.0E11 vg/kg. Three different constructs were tested: AAV8-hOTC-CO1, AAV8-hOTC-CO3, and AAV8-hOTC-CO21. AAV8-hOTC wild-type was used as a control (n=4).

FIG. 27 shows plasma ammonia (NH4) levels of OTC^{spf-ash} mice treated with 5.0E11 vg/kg. Three different constructs were tested: AAV8-hOTC-CO1, AAV8-hOTC-CO3, and AAV8-hOTC-CO21. AAV8-hOTC wild-type and C57Bl/6N wild-type mice were used as controls (n=4).

FIG. 28 shows expression levels of OTC, catalytic activity of OTC, and viral genome copies/cell in OTC^{spf-ash} mice transduced with AAV (5.0E11 vgp/kg). Three different constructs were tested: AAV8-hOTC-CO1, AAV8-hOTC-CO3, and AAV8-hOTC-0006. AAV8-hOTC wild-type and C57Bl/6N wild-type mice were used as controls (n=4, *=P<0.05, **=P<0.01, ***=P<0.001).

FIG. 29 shows expression levels of OTC, catalytic activity of OTC, and viral genome copies/cell in OTC^{spf-ash} mice transduced with AAV (5.0E11 vgp/kg). Two different constructs were tested: AAV8-hOTC-CO1 and AAV8-hOTC-CO3. AAV8-hOTC wild-type was used as a control (n=4).

FIG. 30 shows urinary orotic acid and catalytic activity of OTC^{spf-ash} mice treated with 5.0E11 vg/kg. Two different constructs were tested: AAV8-hOTC-CO1 and AAV8-hOTC-CO3. AAV8-hOTC wild-type and C57Bl/6N wild-type mice were used as controls (n=5).

FIG. 31 shows expression levels of OTC, catalytic activity of OTC, and viral genome copies/cell in OTC^{spf-ash} mice transduced with AAV (1.0E12 vgp/kg). Two different constructs were tested: AAV8-hOTC-CO1 and AAV8-hOTC-CO3. AAV8-hOTC wild-type was used as a control (n=5).

FIG. 32 shows expression levels of OTC, catalytic activity of OTC, and viral genome copies/cell in female OTC^{spf-ash} mice transduced with AAV (5.0E11 vgp/kg). Two different constructs were tested: AAV8-hOTC-CO1 and AAV8-hOTC-CO3. AAV8-hOTC wild-type was used as a control (n=5).

FIG. 33 shows expression levels of OTC, catalytic activity of OTC, and viral genome copies/cell in female OTC^{spf-ash} mice transduced with AAV (1.0E12 vgp/kg). Two different constructs were tested: AAV8-hOTC-CO1 and AAV8-hOTC-CO3. AAV8-hOTC wild-type was used as a control (n=5).

FIG. 34 shows expression levels of OTC, catalytic activity of OTC, and viral genome copies/cell in male OTC^{spf-ash} mice transduced with AAV (1.0E12 vgp/kg). Two different constructs were tested: AAV8-hOTC-CO3 and AAV8-hOTC-CO21. AAV8-hOTC wild-type was used as a control (n=5, *=P<0.05, **=P<0.01)

FIG. 35 shows the urinary orotic acid of OTC^{spf-ash} male mice having the same (1.0E12 vgp/kg) concentration of virus in liver (n=5).

FIG. 36 shows the urinary orotic acid, OTC enzymatic activity, and OTC protein levels in OTC^{spf-ash} mice injected with one of three doses (2.5E11 vgp/kg, 5.0E11 vgp/kg, 1.0E12 vgp/kg) of AAV8-hOTC wild-type or AAV8-hOTC-CO21.

FIG. 37 shows the urinary orotic acid levels in OTC^{spf-ash} male mice treated with 5.0E11 vg/kg AAV8-hOTC-wt or AAV8-hOTC-CO21 (n=5).

FIG. 38 shows protein expression, and catalytic activity of OTC^{spf-ash} male mice treated with 2.5E11 vg/kg of AAV8-hOTC-wt or AAV8-hOTC-CO21 (n=5, *=P<0.05).

FIG. 39 shows protein expression, and catalytic activity of OTC^{spf-ash} male mice treated with 2.5E11 vg/kg of AAV8-hOTC-wt or AAV8-hOTC-CO21 (n=5, *=P<0.05).

FIG. 40 shows the urinary orotic acid in OTC^{spf-ash} male mice treated with 2.5E11 vg/kg of AAV8-OTC-wt or AAV8-hOTC-CO21.

FIG. 41 shows the urinary orotic acid and OTC enzymatic activity in OTC^{spf-ash} male mice treated with one of three doses (2.5E11, 5.0E11, or 1.0E12 vg/kg) of AAV8-hOTC-wt or AAV8-hOTC-CO21.

FIG. 42 shows behavioral test results, plasma ammonia (NH4) levels, and urinary orotic acid levels in OTC^{spf-ash} mice injected with 5E11 vgp/kg AAV8-hOTC wild-type or AAV8-hOTC-CO21 viruses. B6EiC3Sn-WT (WT-CH3) mice were used as a control (n=4, *=P<0.05, **=P<0.01, ***=P<0.001).

FIG. 43 shows the urinary orotic acid of OTC^{spf-ash} mice injected with 5E11 vgp/kg or 1E12 vgp/kg of AAV8-hOTC-CO21.

FIG. 44 shows the behavioral test results, plasma ammonia (NH4) levels, urinary orotic acid levels, protein expression levels, and OTC enzymatic activity in OTC^{spf-ash} mice injected with 5E11 vpg/kg AAV8-hOTC wild-type or AAV8-hOTC-CO21 viruses. Bi6EiC3Sn-WT (WT-CH3) or C57Bl/6N wild-type (C57-WT) mice were used as a control (n=4, *=P<0.05, **=P<0.01, ***=P<0.001).

FIG. 45 shows OTC expression and enzymatic activity in human hepatocytes expressing AAV8-hOTC-CO21 and AAV8-hOTC-Δenhancer-CO21 (AAV8-hOTC-Δ-CO21). Untreated OTC^{spf-ash} mice were used as a control.

FIG. 46 shows the urinary orotic acid and OTC expression of OTC^{spf-ash} mice injected with AAV8-hOTC-CO21 and AAV8-hOTC-Δ-CO21. Untreated OTC_{spf-ash} mice were used as a control.

FIG. 47 shows the urinary orotic acid and anti-AAV8 antibody (Nab) of juvenile (P30) OTC^{spf-ash} mice injected with 5.0E11 vgp/kg AAV8-hOTC-CO21 virus. Untreated OTC^{spf-ash} mice were used as a control.

FIG. 48 shows the levels of anti-AAV8 IgG antibody and the expression of hFIX in C57BL/6 mice injected with 4.0E12 vg/kg AAV8-luciferase (AAV8-luc) and 8 mg/kg SVP [Rapa] or SVP [empty], followed by 4.0E12 vg/kg AAV8-hFIX and 8 vg/kg SVP [Rapa] or SVP [empty] (n=5/group).

FIG. 49 shows the levels of anti-AAV8 IgG antibody and expression of hFIX in Macaca fascicularis non-human primates injected with 2.0E12 vg/kg AAV8-Gaa and 3 mg/kg SVP [Rapa] or SVP [empty], followed by 2.0E12 vg/kg AAV8-hFIX and 3 mg/kg SVP [Rapa] or SVP [empty] (n=2 SVP[Rapa]+AAV, n=1 SVP[Empty]+AAV).

FIG. 50 shows the level of anti-AAV8 IgG antibody in OTC^{spf-ash} mice two weeks after injection with AAV8-OTC CO21 alone (“AAV”, closed circles), AAV8-OTC CO21+empty nanoparticle control (“AAV+NPc”, closed squares), AAV8-OTC CO21+4 mg/kg SVP-Rapamycin (“AAV+SVP4”, closed triangles), AAV8-OTC CO21+8 mg/kg SVP-Rapamycin (“AAV+SVP8”, inverted closed triangles), or AAV8-OTC CO21+12 mg/kg SVP-Rapamycin (“AAV+SVP12”, closed diamonds).

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes. Such incorporation by reference is not intended to be an admission that any of the incorporated publications, patents and patent applications cited herein constitute prior art.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to “a DNA molecule” includes a mixture of two or more such DNA molecules or a plurality of such DNA molecules, reference to “an immunosuppressant” includes a mixture of two or more such immunosuppressant molecules or a plurality of such immunosuppressant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### A. INTRODUCTION

Urea cycle defects (UCDs) are generally caused by genetic disorders resulting in a deficiency of one of the six enzymes in the urea cycle, leading to an accumulation of ammonia in blood. Despite dietary protein restriction and ammonia scavenging drugs, children with UCDs develop disabilities related to hyperammonemia, and many die in the first two decades of life. There is no definitive treatment for the most common UCD, ornithine transcarbamylase deficiency (OTCd), apart from liver transplantation, an invasive procedure limited by organ availability and life-long immunosuppression treatment. Ornithine TransCarbamylase deficiency (OTCd) is a monogenic, X-linked, urea cycle disease with an estimated prevalence of 15,000-60,000 live births. The most severe OTC deficiency patients manifest symptoms immediately after birth, with severe ammonia crisis that can lead to coma and premature death. A second group of patients is characterized by a late onset manifestation, including delayed development and intellectual disability, due to a partial residual activity of the enzyme (Campbell et al., 1973; Wraith, 2001; Gordon, 2003).

As examples, a series of ssAAV vector constructs expressing human OTC transgene under the transcriptional control of a liver-specific promoter were developed. The wt-hOTC was Codon-Optimized (CO) with different algorithms. These candidate vectors were packaged into AAV8 and used to transduce OTCspf-ash (5×10¹¹ and 1×10¹² vgp/kg) mice. By measuring the number of viral genome copies per cell, protein levels, catalytic activity, urinary orotic acid levels, and plasma ammonia levels, CO-hOTC constructs that were particularly efficient in correcting the phenotype of OTCspf-ash mice were identified. Compositions comprising such constructs are provided herein in some aspects. Such constructs can be used in any one of the methods and compositions provided herein.

In addition, it is noted that while viral vectors are promising therapeutics for a variety of applications such as transgene expression, cellular and humoral immune responses against the viral vector can diminish efficacy and/or reduce the ability to use such therapeutics in a repeat administration context. These immune responses include antibody, B cell and T cell responses and can be specific to viral antigens of the viral vector, such as viral capsid or coat proteins or peptides thereof.

It has been found that adeno-associated virus (AAV) vectors encoding the OTC gene for administration in combination with biodegradable synthetic nanocarriers containing an immunosuppressant, such as rapamycin, can be made and used to prevent immune responses, such as antibody responses, for example to an immunogenic therapeutic enzyme. In studies, the synthetic nanocarriers comprising immunosuppressant blocked humoral and cellular immune responses to AAV, which for OTCd could have two benefits: 1) ability to treat patients at an early age, while maintaining the possibility to re-dose later in life to maintain therapeutic expression levels, and 2) minimize use of steroids, which may trigger metabolic crisis. Thus, provided herein are methods and compositions for treating a subject with a recombinant AAV vector comprising any one of the constructs provided herein in combination with synthetic nanocarriers comprising an immunosuppressant.

Thus, the inventors have surprisingly and unexpectedly discovered that the problems and limitations noted above can be overcome by practicing the invention disclosed herein. Methods and compositions are provided that offer solutions to the aforementioned obstacles to effective use of the viral vectors for treatment.

The invention will now be described in more detail below.

### B. DEFINITIONS

“Additional therapeutic” refers to any therapeutic agent that is in addition to the viral vector and/or synthetic nanocarriers comprising an immunosuppressant. In some embodiments, the additional therapeutic is a steroid, such as a corticosteroid.

“Administering” or “administration” or “administer” means giving or dispensing a material to a subject in a manner that is pharmacologically useful. The term is intended to include “causing to be administered”. “Causing to be administered” means causing, urging, encouraging, aiding, inducing or directing, directly or indirectly, another party to administer the material. Any one of the methods provided herein may comprise or further comprise a step of administering concomitantly an AAV vector and synthetic nanocarriers comprising an immunosuppressant. In some embodiments, the concomitant administration is performed repeatedly. In still further embodiments, the concomitant administration is simultaneous administration. “Simultaneous” means administration at the same time or substantially at the same time where a clinician would consider any time between administrations virtually nil or negligible as to the impact on the desired therapeutic outcome. In some embodiments, simultaneous means that the administrations occur with 5, 4, 3, 2, 1 or fewer minutes.

“Amount effective” in the context of a composition or dosage form for administration to a subject as provided herein refers to an amount of the composition or dosage form that produces one or more desired results in the subject, for example, the reduction or elimination of an immune response against a viral vector or an expression product thereof and/or efficacious transgene expression. The amount effective can be for in vitro or in vivo purposes. For in vivo purposes, the amount can be one that a clinician would believe may have a clinical benefit for a subject. In any one of the methods provided herein, the composition(s) administered may be in any one of the amounts effective as provided herein.

Amounts effective can involve reducing the level of an undesired immune response, although in some embodiments, it involves preventing an undesired immune response altogether. Amounts effective can also involve delaying the occurrence of an undesired immune response. An amount effective can also be an amount that results in a desired therapeutic endpoint or a desired therapeutic result. Amounts effective, in some embodiments, result in a tolerogenic immune response in a subject to an antigen, such as a viral antigen of the viral vector and/or expressed product. Amounts effective also can result in increased transgene expression (the transgene being delivered by the viral vector). This can be determined by measuring transgene protein concentrations in various tissues or systems of interest in the subject. This increased expression may be measured locally or systemically. The achievement of any of the foregoing can be monitored by routine methods.

In some embodiments of any one of the compositions and methods provided, the amount effective is one in which the desired immune response, such as the reduction or elimination of an immune response, persists in the subject for at least 1 week, at least 2 weeks or at least 1 month. In other embodiments of any one of the compositions and methods provided, the amount effective is one which produces a measurable desired immune response, such as the reduction or elimination of an immune response. In some embodiments, the amount effective is one that produces a measurable desired immune response, for at least 1 week, at least 2 weeks or at least 1 month.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation.

“Attach” or “Attached” or “Couple” or “Coupled” (and the like) means to chemically associate one entity (for example a moiety) with another. In some embodiments, the attaching is covalent, meaning that the attachment occurs in the context of the presence of a covalent bond between the two entities. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of attaching.

“Average”, as used herein, refers to the arithmetic mean unless otherwise noted.

“Codon-optimized” refers to optimization of a nucleic acids sequence encoding a protein by changing codons generally without resulting in a change in the amino acid sequence but resulting in increased or more efficient expression. Codon-optimization is a technique used to improve protein expression of a protein coding gene, e.g., OTC, in an organism by increasing the transcriptional and translational efficiency of the gene. Decreased protein expression of a target gene in a living organism can be due to numerous factors, including, but not limited to: the presence of rare codons, GC content, mRNA structure, repeated sequences, and the presence of restriction enzyme cleavage sites. Different codon-optimization algorithms consider and weigh these factors to varying levels. Typically, multiple different codon-optimization algorithms will be used for a particular sequence and compared side-by-side.

In some embodiments, codon-optimization is be performed to alter the sequence of codons in a nucleic acid sequence, e.g., an mRNA sequence. In some embodiments, the a nucleic acid sequence is altered without altering the encoded amino acid sequence. Codons are 3 base pair blocks of a nucleotide sequence in an mRNA that are bound by a complementary transfer RNA (tRNA) during RNA translation into protein. In some instances, an mRNA sequence is altered to remove a rare codon. Rare codons codons are complementary to a tRNA that is either not present or is present at low levels in an organism in which the target gene is expressedThe presence of rare codons in a target gene can decrease or even block protein translation. In some embodiments, changing the nucleic acid sequence to remove a rare codons for a given organism without changing the amino acid sequence may improve protein expression.

In some instances, a nucleic acid sequence, e.g., an mRNA sequence is altered to increase or decrease the GC content of the nucleic acid sequence. The guanosine/cytosine (GC) content of a nucleic acid sequence is the percentage of nucleotides in the nucleic acid sequence that are G or C. Guanosine and cytosine are complementary and form 3 hydrogen bonds in double-stranded nucleotides, while adenine and thymine or adenine and uracil only form 2 hydrogen bonds. This increase in the number of hydrogen bonds increases the stability of the nucleic acid molecule. In some embodiments, changing the nucleic acid sequence to increase the GC content without changing the amino acid sequence may improve protein expression. In some embodiments, changing the nucleic acid sequence to decrease the GC content without changing the amino acid sequence may improve protein expression.

The structure of mRNA plays a critical role in regulating translation of mRNA into protein in an organism. When mRNA forms a secondary, tertiary, or quaternary structure, these structures may render the codons inaccessible to binding by tRNAs or ribosomes, inhibiting translation. Secondary and tertiary structures of mRNAs include stem loops and pseudoknots, with tertiary structures being more complex, three-dimensional mRNA forms than secondary structures. Quaternary structures of mRNAs include mRNA-mRNA homodimers and mRNA-mRNA heterodimers. In some embodiments, changing the a nucleic acid sequence, e.g., an mRNA sequence, to decrease or avoid the formation of mRNA secondary, tertiary, or quaternary structures without changing the amino acid sequence may improve protein expression.

In some embodiments, the presence of repeated sequences in a nucleic acid sequence, e.g., an mRNA sequence, decreases protein expression by inhibiting transcription and translation of the target gene. Repeated sequences decrease transcription and translation by exhausting available nucleotide and tRNA pools. Additionally, repeated sequences may also decrease translation by allowing formation of mRNA secondary and tertiary structures. In some embodiments, changing the nucleic acid sequence to remove or reduced repeated sequences without changing the amino acid sequence may improve protein expression.

In some embodiments, the presence of restriction enzyme cleavage sites in a nucleic acid sequence, e.g., an mRNA sequence, decreases protein expression by inhibiting transcription and translation of the nucleic acid, e.g., the mRNA. Restriction enzymes are proteins that cleave nucleic acids after binding at specific sequences. These cleaved nucleic acids may not be suitable substrates for transcription or translation. In some embodiments, changing the nucleic acid sequence to remove restriction enzyme cleavage sites without changing the amino acid sequence improves protein expression.

“Concomitantly” means administering two or more materials/agents to a subject in a manner that is correlated in time, preferably sufficiently correlated in time so as to provide a modulation in an immune response, and even more preferably the two or more materials/agents are administered in combination. In embodiments, concomitant administration may encompass administration of two or more materials/agents within a specified period of time, preferably within 1 month, more preferably within 1 week, still more preferably within 1 day, and even more preferably within 1 hour. In embodiments, the materials/agents may be repeatedly administered concomitantly; that is concomitant administration on more than one occasion.

“Dose” refers to a specific quantity of a pharmacologically and/or immunologically active material for administration to a subject for a given time. In general, doses of the synthetic nanocarriers comprising an immunosuppressant and/or viral vectors in the methods and compositions of the invention refer to the amount of the synthetic nanocarriers comprising an immunosuppressant and/or viral vectors. Alternatively, the dose can be administered based on the number of synthetic nanocarriers that provide the desired amount of an immunosuppressant, in instances when referring to a dose of synthetic nanocarriers that comprise an immunosuppressant. When dose is used in the context of a repeated dosing, dose refers to the amount of each of the repeated doses, which may be the same or different.

“Early disease onset” refers to the onset of the disease in a subject at an age that is earlier than the average age of disease onset or earlier than the expected age of disease onset. In some embodiments, early disease onset occurs in childhood. Early disease onset can be determined by a clinician.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier.

“Expression control sequences” are any sequences that can affect expression and can include promoters, enhancers, and operators. In one embodiment of any one of the methods or compositions provided, the expression control sequence is a promoter. In one embodiment of any one of the methods or compositions provided, the expression control sequence is a liver-specific promoter. “Liver-specific promoters” are those that exclusively or preferentially result in expression in cells of the liver.

“Identity” means the percentage of amino acid or residues or nucleic acid bases that are identically positioned in a one-dimensional sequence alignment. Identity is a measure of how closely the sequences being compared are related. In an embodiment, identity between two sequences can be determined using the BESTFIT program. Additionally, the percent identity can also be calculated using various, publicly available software tools developed by NCBI (Bethesda, Md.) that can be obtained through the internet (ftp:/ncbi.nlm.nih.gov/pub/). Exemplary tools include the BLAST system available at http://wwww.ncbi.nlm.nih.gov. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements (including full-length complements) of the foregoing nucleic acids also are embraced by the invention. “Immunosuppressant” means a compound that can cause a tolerogenic effect, preferably through its effects on APCs. A tolerogenic effect generally refers to the modulation by the APC or other immune cells systemically and/or locally, that reduces, inhibits or prevents an undesired immune response to an antigen in a durable fashion. In one embodiment, the immunosuppressant is one that causes an APC to promote a regulatory phenotype in one or more immune effector cells. For example, the regulatory phenotype may be characterized by the inhibition of the production, induction, stimulation or recruitment of antigen-specific CD4+ T cells or B cells, the inhibition of the production of antigen-specific antibodies, the production, induction, stimulation or recruitment of Treg cells (e.g., CD4+CD25highFoxP3+ Treg cells), etc. This may be the result of the conversion of CD4+ T cells or B cells to a regulatory phenotype. This may also be the result of induction of FoxP3 in other immune cells, such as CD8+ T cells, macrophages and iNKT cells. In one embodiment, the immunosuppressant is one that affects the response of the APC after it processes an antigen. In another embodiment, the immunosuppressant is not one that interferes with the processing of the antigen. In a further embodiment, the immunosuppressant is not an apoptotic-signaling molecule. In another embodiment, the immunosuppressant is not a phospholipid.

Immunosuppressants include, but are not limited to, statins; mTOR inhibitors, such as rapamycin or a rapamycin analog (i.e., rapalog); TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, retinoic acid, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide. Other exemplary immunosuppressants include, but are not limited, small molecule drugs, natural products, antibodies (e.g., antibodies against CD20, CD3, CD4), biologics-based drugs, carbohydrate-based drugs, RNAi, antisense nucleic acids, aptamers, methotrexate, NSAIDs; fingolimod; natalizumab; alemtuzumab; anti-CD3; tacrolimus (FK506), abatacept, belatacept, etc. “Rapalog”, as used herein, refers to a molecule that is structurally related to (an analog) of rapamycin (sirolimus). Examples of rapalogs include, without limitation, temsirolimus (CCI-779), everolimus (RAD001), ridaforolimus (AP-23573), and zotarolimus (ABT-578). Additional examples of rapalogs may be found, for example, in WO Publication WO 1998/002441 and U.S. Pat. No. 8,455,510, the rapalogs of which are incorporated herein by reference in their entirety.

The immunosuppressant can be a compound that directly provides the tolerogenic effect on APCs or it can be a compound that provides the tolerogenic effect indirectly (i.e., after being processed in some way after administration). Further immunosuppressants, are known to those of skill in the art, and the invention is not limited in this respect. In embodiments, the immunosuppressant may comprise any one of the agents provided herein.

“Load”, when coupled to a synthetic nanocarrier, is the amount of the immunosuppressant coupled to the synthetic nanocarrier based on the total dry recipe weight of materials in an entire synthetic nanocarrier (weight/weight). Generally, such a load is calculated as an average across a population of synthetic nanocarriers. In one embodiment, the load on average across the synthetic nanocarriers is between 0.1% and 99%. In another embodiment, the load is between 0.1% and 50%. In another embodiment, the load is between 0.1% and 20%. In a further embodiment, the load is between 0.1% and 10%. In still a further embodiment, the load is between 1% and 10%. In still a further embodiment, the load is between 7% and 20%. In yet another embodiment, the load is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 40%, at least 50%, at least 60%, at least 70%, at least 80%, at least 90%, at least 95%, at least 96%, at least 97%, at least 98% or at least 99% on average across the population of synthetic nanocarriers. In yet a further embodiment, the load is 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20% on average across the population of synthetic nanocarriers. In some embodiments of the above embodiments, the load is no more than 25% on average across a population of synthetic nanocarriers. In embodiments, the load is calculated as may be described in the Examples or as otherwise known in the art.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1.

Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., effective diameter) may be obtained, in some embodiments, by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicia of the sample. The effective diameter, or mean of the distribution, is then reported. Determining the effective sizes of high aspect ratio, or non-spheroidal, synthetic nanocarriers may require augmentative techniques, such as electron microscopy, to obtain more accurate measurements. “Dimension” or “size” or “diameter” of synthetic nanocarriers means the mean of a particle size distribution, for example, obtained using dynamic light scattering.

“Pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Polynucleotide(s)” or “nucleic acid sequence(s)” or “nucleic acid(s)” are used interchangeably herein and may be, for example, DNA, RNA (such as, for example, mRNA) or cDNA. The AAV vectors and transgenes described herein comprise polynucleotides. In some embodiments, the polynucleotides encode the transgene, e.g., OTC.

In embodiments, the inventive compositions comprise a complement, such as a full-length complement, or a degenerate (due to degeneracy of the genetic code) encoding any of the polypeptides of the present invention.

Also provided herein are polynucleotides that hybridize to any of the polynucleotides of the present invention. Standard nucleic acid hybridization procedures can be used to identify related nucleic acid sequences of selected percent identity. The term “stringent conditions” as used herein refers to parameters with which the art is familiar. Such parameters include salt, temperature, length of the probe, etc. The amount of resulting base mismatch upon hybridization can range from near 0% (“high stringency”) to about 30% (“low stringency”). One example of high-stringency conditions is hybridization at 65° C. in hybridization buffer (3.5×SSC, 0.02% Ficoll, 0.02% polyvinyl pyrrolidone, 0.02% Bovine Serum Albumin, 2.5 mM NaH2PO4(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68° C.

“Repeat dose” or “repeat dosing” or the like means at least one additional dose or dosing that is administered to a subject subsequent to an earlier dose or dosing of the same material. For example, a repeated dose of a viral vector is at least one additional dose of the viral vector after a prior dose of the same material. While the material may be the same, the amount of the material in the repeated dose may be different from the earlier dose. For example, in an embodiment of any one of the methods or compositions provided herein, the amount of the viral vector in the repeated dose may be less than the amount of the viral vector of the earlier dose. Alternatively, in an embodiment of any one of the methods or compositions provided herein, the repeated dose may be in an amount that is at least equal to the amount of the viral vector in the earlier dose. A repeat dose may be administered weeks, months or years after the prior dose. In some embodiments of any one of the methods provided herein, the repeat dose or dosing is administered at least 1 week after the dose or dosing that occurred just prior to the repeat dose or dosing. Repeat dosing is considered to be efficacious if it results in a beneficial effect for the subject. Preferably, efficacious repeat dosing results in a beneficial effect in conjunction with reduced immune response, such as to the viral vector.

A “reduced amount” refers to a dose of a therapeutic that is less than the amount of the therapeutic that has been administered, such as in a prior administration, to a subject or that would be selected for administration to the subject without the concomitant administration of an AAV vector and synthetic nanocarriers comprising an immunosuppressant as provided herein. In some embodiments of any one of the methods provided herein, the method may comprise or further comprise a step of selecting a reduced amount of a therapeutic as described herein. “Selecting” is intended to include “causing to select”. “Causing to select” means causing, urging, encouraging, aiding, inducing or directing or acting in coordination with an entity for the entity to select the aforementioned reduced amount.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like. As used herein, a subject may be in one need of any one of the methods or compositions provided herein. In some embodiments, a subject has or is suspected of having a UCD, e.g., OTCd. In some embodiments, a subject is at risk of developing a UCD, e.g., OTCd. In some embodiments, the subject is a pediatric or juvenile subject, e.g., is less than 18, less than 16, less than 15, less than 14, less than 13, less than 12, less than 11, less than 10, less than 9, less than 8, less than 7, less than 6, less than 5, less than 4, less than 3 years old, or less than 2 years old. In some embodiments, the subject is 1-10 years old. In some embodiments, the subject is an adult subject.

“Synthetic nanocarrier(s)” means a discrete object that is not found in nature, and that possesses at least one dimension that is less than or equal to 5 microns in size. Albumin nanoparticles are generally included as synthetic nanocarriers; however in certain embodiments the synthetic nanocarriers do not comprise albumin nanoparticles. In embodiments, synthetic nanocarriers do not comprise chitosan. In other embodiments, synthetic nanocarriers are not lipid-based nanoparticles. In further embodiments, synthetic nanocarriers do not comprise a phospholipid.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid attached virus-like particles disclosed in published US Patent Application 20060251677 to Bachmann et al., (9) the virus-like particles disclosed in WO2010047839A1 or WO2009106999A2, (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), (11) apoptotic cells, apoptotic bodies or the synthetic or semisynthetic mimics disclosed in U.S. Publication 2002/0086049, or (12) those of Look et al., Nanogel-based delivery of mycophenolic acid ameliorates systemic lupus erythematosus in mice” J. Clinical Investigation 123(4):1741-1749(2013).

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, synthetic nanocarriers may possess an aspect ratio greater than 1:1, 1:1.2, 1:1.5, 1:2, 1:3, 1:5, 1:7, or greater than 1:10.

“Urea cycle disorder” refers to any disorder or defect whereby there is a deficiency of an enzyme of the urea cycle. Generally, this is caused by a mutation that results in such a deficiency in a subject. Thus, an “enzyme associated with the urea cycle disorder” is an enzyme in which there is a deficiency that results in the disorder in the subject.

“Viral vector” means a vector construct with viral components, such as capsid and/or coat proteins, that has been adapted to comprise and deliver a transgene or nucleic acid material that encodes therapeutic, such as a therapeutic protein, which transgene or nucleic acid material can be expressed as provided herein. “Expressed” or “expression” or the like refers to the synthesis of a functional (i.e., physiologically active for the desired purpose) product after the transgene or nucleic acid material is transduced into a cell and processed by the transduced cell. Such a product is also referred to herein as an “expression product”. Viral vectors can be based on, without limitation, adeno-associated viruses, such as AAV8. Thus, an AAV vector provided herein is a viral vector based on an AAV, such as AAV8, and has viral components, such as a capsid and/or coat protein, therefrom that can package for delivery the transgene or nucleic acid material.

### C. COMPOSITIONS FOR USE IN THE INVENTIVE METHODS

As mentioned above, there is no definitive treatment for the most common UCD, ornithine transcarbamylase deficiency (OTCd), apart from liver transplantation, an invasive procedure limited by organ availability and life-long immunosuppression treatment. In addition, also as mentioned above, immune responses, such as humoral and cellular immune responses, against a viral vector can adversely impact its efficacy and can also interfere with its readministration. Importantly, the methods and compositions provided herein have been found to overcome the aforementioned obstacles by achieving strong expression of OTC and/or reducing immune responses to viral vectors encoding OTC, such as encoded by a codon-optimized sequence.

### Transgenes

The transgene or nucleic acid material, such as of the viral vectors, provided herein may encode any protein or portion thereof beneficial to a subject, such as one with a disease or disorder. Generally, the subject has or is suspected of having a disease or disorder whereby the subject's endogenous version of the protein is defective or produced in limited amounts or not at all. The subject may be one with any one of the diseases or disorders as provided herein, and the transgene or nucleic acid material is one that encodes any one of the therapeutic proteins or portion thereof as provided herein. In some embodiments, the transgene may be codon-optimized. The transgene or nucleic acid material provided herein may encode a functional version of any protein that through some defect in the endogenous version of which in a subject (including a defect in the expression of the endogenous version) results in a disease or disorder in the subject. Examples of such diseases or disorders include, but are not limited to, urea cycle enzyme defects, such as ornithine transcarbamylase synthetase deficiency (OTCd). It follows that therapeutic proteins encoded by the transgene or nucleic acid material includes ornithine transcarbamylase synthetase (OTC).

The sequence of a transgene or nucleic acid material may also include an expression control sequence. Expression control sequences include promoters, enhancers, and operators, and are generally selected based on the expression systems in which the expression construct is to be utilized. In some embodiments, promoter and enhancer sequences are selected for the ability to increase gene expression, while operator sequences may be selected for the ability to regulate gene expression. The transgene may also include sequences that facilitate, and preferably promote, homologous recombination in a host cell. The transgene may also include sequences that are necessary for replication in a host cell.

Exemplary expression control sequences include liver-specific promoter sequences, such as any one that may be provided herein. Generally, promoters are operatively linked upstream (i.e., 5′) of the sequence coding for a desired expression product. The transgene also may include a suitable polyadenylation sequence operably linked downstream (i.e., 3′) of the coding sequence.

Exemplary transgene sequences contemplated by this disclosure are presented in Table 1, following the Examples section. In some embodiments, the transgene sequence may be identical to one or more of the nucleic sequences in Table 1. The transgene sequence in some embodiments that of CO3 or CO21 as provided herein.

In some embodiments, the transgene sequence is a nucleic acid sequence that is at least 60%, at least 65%, at least 70%, at least 75%, at least 80%, at least 85%, at least 90%, at least 95%, at least 97%, or at least 99% identity to any one of the nucleic acid sequences of SEQ ID NO:1-13 (Table 1). Polynucleotides that encode these polypeptides are also contemplated as part embodiments of the present invention. In some embodiments, the transgene sequence is 90%, 91%, 92%, 93%, 94%, 95%, 96%, 97%, 98%, or 99% identical to one or more of the transgene sequences provided herein, such as that of CO3 or CO21.

In some embodiments, the transgene sequence encodes a polypeptide that is identical to one or more of the amino acid sequences in Table 1, e.g., SEQ ID NOs. 14-25. In some embodiments, the transgene sequence encodes an amino acid sequence that is at least 60%, at least 65%, at least 70%, at least 75%, at least 80%, at least 85%, at least 90%, at least 95%, at least 97%, or at least 99% identity to any one of the amino acid sequences of SEQ ID NO: 14-25 (Table 1).

Nucleic acids comprising any one of the sequences provided herein, or a portion thereof that encodes an OTC, is provided in one aspect. Compositions of such nucleic acids are also provided.

### Viral Vectors

Viruses have evolved specialized mechanisms to transport their genomes inside the cells that they infect; viral vectors based on such viruses can be tailored to transduce cells to specific applications. Examples of viral vectors that may be used as provided herein are known in the art or described herein. Suitable viral vectors include, for instance, adeno-associated virus (AAV)-based vectors.

The viral vectors provided herein can be based on adeno-associated viruses (AAVs). AAV vectors have been of particular interest for use in therapeutic applications such as those described herein. AAV is a DNA virus, which is not known to cause human disease. Generally, AAV requires co-infection with a helper virus (e.g., an adenovirus or a herpes virus), or expression of helper genes, for efficient replication. For a description of AAV-based vectors, see, for example, U.S. Pat. Nos. 8,679,837, 8,637,255, 8,409,842, 7,803,622, and 7,790,449, and U.S. Publication Nos. 20150065562, 20140155469, 20140037585, 20130096182, 20120100606, and 20070036757. The AAV vectors may be recombinant AAV vectors. The AAV vectors may also be self-complementary (sc) AAV vectors, which are described, for example, in U.S. Patent Publications 2007/01110724 and 2004/0029106, and U.S. Pat. Nos. 7,465,583 and 7,186,699.

The adeno-associated virus on which a viral vector is based may be of a specific serotype, such as AAV8. In some embodiments of any one of the methods or compositions provided herein, therefore, the AAV vector is an AAV8 vector.

### Synthetic Nanocarriers Comprising an Immunosuppressant

The viral vectors provided herein can be administered in combination with synthetic nanocarriers comprising an immunosuppressant. Generally, the immunosuppressant is an element that is in addition to the material that makes up the structure of the synthetic nanocarrier. For example, in one embodiment, where the synthetic nanocarrier is made up of one or more polymers, the immunosuppressant is a compound that is in addition and, in some embodiments, attached to the one or more polymers. In embodiments where the material of the synthetic nanocarrier also results in a tolerogenic effect, the immunosuppressant is an element present in addition to the material of the synthetic nanocarrier that results in a tolerogenic effect.

A wide variety of other synthetic nanocarriers can be used according to the invention, and in some embodiments, coupled to an immunosuppressant. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size or shape so that each synthetic nanocarrier has similar properties. For example, at least 80%, at least 90%, or at least 95% of the synthetic nanocarriers of any one of the compositions or methods provided, based on the total number of synthetic nanocarriers, may have a minimum dimension or maximum dimension that falls within 5%, 10%, or 20% of the average diameter or average dimension of the synthetic nanocarriers.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, synthetic nanocarriers can comprise one or more polymers. In some embodiments, the synthetic nanocarriers comprise one or more polymers that is a non-methoxy-terminated, pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated, pluronic polymers. In some embodiments, all of the polymers that make up the synthetic nanocarriers are non-methoxy-terminated, pluronic polymers. In some embodiments, the synthetic nanocarriers comprise one or more polymers that is a non-methoxy-terminated polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated polymers. In some embodiments, all of the polymers that make up the synthetic nanocarriers are non-methoxy-terminated polymers. In some embodiments, the synthetic nanocarriers comprise one or more polymers that do not comprise pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers do not comprise pluronic polymer. In some embodiments, all of the polymers that make up the synthetic nanocarriers do not comprise pluronic polymer. In some embodiments, such a polymer can be surrounded by a coating layer (e.g., liposome, lipid monolayer, micelle, etc.). In some embodiments, elements of the synthetic nanocarriers can be attached to the polymer.

Immunosuppressants can be coupled to the synthetic nanocarriers by any of a number of methods. Generally, the attaching can be a result of bonding between the immunosuppressants and the synthetic nanocarriers. This bonding can result in the immunosuppressants being attached to the surface of the synthetic nanocarriers and/or contained (encapsulated) within the synthetic nanocarriers. In some embodiments, however, the immunosuppressants are encapsulated by the synthetic nanocarriers as a result of the structure of the synthetic nanocarriers rather than bonding to the synthetic nanocarriers. In preferable embodiments, the synthetic nanocarrier comprises a polymer as provided herein, and the immunosuppressants are attached to the polymer.

When attaching occurs as a result of bonding between the immunosuppressants and synthetic nanocarriers, the attaching may occur via a coupling moiety. A coupling moiety can be any moiety through which an immunosuppressant is bonded to a synthetic nanocarrier. Such moieties include covalent bonds, such as an amide bond or ester bond, as well as separate molecules that bond (covalently or non-covalently) the immunosuppressant to the synthetic nanocarrier. Such molecules include linkers or polymers or a unit thereof. For example, the coupling moiety can comprise a charged polymer to which an immunosuppressant electrostatically binds. As another example, the coupling moiety can comprise a polymer or unit thereof to which it is covalently bonded.

In preferred embodiments, the synthetic nanocarriers comprise a polymer as provided herein. These synthetic nanocarriers can be completely polymeric or they can be a mix of polymers and other materials.

In some embodiments, the polymers of a synthetic nanocarrier associate to form a polymeric matrix. In some of these embodiments, a component, such as an immunosuppressant, can be covalently associated with one or more polymers of the polymeric matrix. In some embodiments, covalent association is mediated by a linker. In some embodiments, a component can be noncovalently associated with one or more polymers of the polymeric matrix. For example, in some embodiments, a component can be encapsulated within, surrounded by, and/or dispersed throughout a polymeric matrix. Alternatively or additionally, a component can be associated with one or more polymers of a polymeric matrix by hydrophobic interactions, charge interactions, van der Waals forces, etc. A wide variety of polymers and methods for forming polymeric matrices therefrom are known conventionally.

Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments, the polymer comprises a polyester, polycarbonate, polyamide, or polyether, or unit thereof. In other embodiments, the polymer comprises poly(ethylene glycol) (PEG), polypropylene glycol, poly(lactic acid), poly(glycolic acid), poly(lactic-co-glycolic acid), or a polycaprolactone, or unit thereof. In some embodiments, it is preferred that the polymer is biodegradable. Therefore, in these embodiments, it is preferred that if the polymer comprises a polyether, such as poly(ethylene glycol) or polypropylene glycol or unit thereof, the polymer comprises a block-co-polymer of a polyether and a biodegradable polymer such that the polymer is biodegradable. In other embodiments, the polymer does not solely comprise a polyether or unit thereof, such as poly(ethylene glycol) or polypropylene glycol or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids. Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids. In embodiments, the synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

Any immunosuppressant as provided herein can be, in some embodiments, coupled to synthetic nanocarriers. Immunosuppressants include, but are not limited to, statins; mTOR inhibitors, such as rapamycin or a rapamycin analog (rapalog); TGF-β signaling agents; TGF-β receptor agonists; histone deacetylase (HDAC) inhibitors; corticosteroids; inhibitors of mitochondrial function, such as rotenone; P38 inhibitors; NF-κβ inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cyclosporine A, siRNAs targeting cytokines or cytokine receptors and the like.

Examples of mTOR inhibitors include rapamycin and analogs thereof (e.g., CCL-779, RAD001, AP23573, C20-methallylrapamycin (C20-Marap), C16-(S)-butylsulfonamidorapamycin (C16-BSrap), C16-(S)-3-methylindolerapamycin (C16-iRap) (Bayle et al. Chemistry & Biology 2006, 13:99-107)), AZD8055, BEZ235 (NVP-BEZ235), chrysophanic acid (chrysophanol), deforolimus (MK-8669), everolimus (RAD0001), KU-0063794, PI-103, PP242, temsirolimus, and WYE-354 (available from Selleck, Houston, Tex., USA).

Compositions according to the invention can comprise pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, compositions are suspended in sterile saline solution for injection together with a preservative.

### D. METHODS OF USING AND MAKING THE COMPOSITIONS AND RELATED METHODS

Viral vectors can be made with methods known to those of ordinary skill in the art or as otherwise described herein. For example, viral vectors can be constructed and/or purified using the methods set forth, for example, in U.S. Pat. No. 4,797,368 and Laughlin et al., Gene, 23, 65-73 (1983).

AAV vectors may be produced using recombinant methods. Typically, the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, the viral vector may comprise inverted terminal repeats (ITR) of AAV serotypes, such as AAV8.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell can contain the required component(s) under the control of an inducible promoter. However, the required component(s) may be under the control of a constitutive promoter. The recombinant AAV vector, rep sequences, cap sequences, and helper functions required for producing the rAAV of the invention may be delivered to the packaging host cell using any appropriate genetic element. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

In some embodiments, recombinant AAV vectors may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (comprising a transgene) to be packaged into AAV particles, an AAV helper function vector, and an accessory function vector. Generally, an AAV helper function vector encodes AAV helper function sequences (rep and cap), which function in trans for productive AAV replication and encapsidation. Preferably, the AAV helper function vector supports efficient AAV vector production without generating any detectable wild-type AAV virions (i.e., AAV virions containing functional rep and cap genes). The accessory function vector can encode nucleotide sequences for non-AAV derived viral and/or cellular functions upon which AAV is dependent for replication. The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Other methods for producing viral vectors are known in the art. Moreover, viral vectors are available commercially.

In regard to synthetic nanocarriers coupled to immunosuppressants, methods for attaching components to synthetic nanocarriers may be useful.

In certain embodiments, the attaching can be via a covalent linker. In embodiments, immunosuppressants according to the invention can be covalently attached to the external surface via a 1,2,3-triazole linker formed by the 1,3-dipolar cycloaddition reaction of azido groups with immunosuppressant containing an alkyne group or by the 1,3-dipolar cycloaddition reaction of alkynes with immunosuppressants containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, or a sulfonamide linker.

An amide linker is formed via an amide bond between an amine on one component such as an immunosuppressant with the carboxylic acid group of a second component such as the nanocarrier. The amide bond in the linker can be made using any of the conventional amide bond forming reactions with suitably protected amino acids and activated carboxylic acid such N-hydroxysuccinimide-activated ester.

A disulfide linker is made via the formation of a disulfide (S—S) bond between two sulfur atoms of the form, for instance, of R1-S—S—R2. A disulfide bond can be formed by thiol exchange of a component containing thiol/mercaptan group(—SH) with another activated thiol group or a component containing thiol/mercaptan groups with a component containing activated thiol group.

A triazole linker, specifically a 1,2,3-triazole of the form

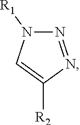

wherein R1 and R2 may be any chemical entities, is made by the 1,3-dipolar cycloaddition reaction of an azide attached to a first component with a terminal alkyne attached to a second component such as the immunosuppressant. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

A thioether linker is made by the formation of a sulfur-carbon (thioether) bond in the form, for instance, of R1-S—R2. Thioether can be made by either alkylation of a thiol/mercaptan (—SH) group on one component with an alkylating group such as halide or epoxide on a second component. Thioether linkers can also be formed by Michael addition of a thiol/mercaptan group on one component to an electron-deficient alkene group on a second component containing a maleimide group or vinyl sulfone group as the Michael acceptor. In another way, thioether linkers can be prepared by the radical thiol-ene reaction of a thiol/mercaptan group on one component with an alkene group on a second component.

A hydrazone linker is made by the reaction of a hydrazide group on one component with an aldehyde/ketone group on the second component.

A hydrazide linker is formed by the reaction of a hydrazine group on one component with a carboxylic acid group on the second component. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component with an aldehyde or ketone group on the second component.

An urea or thiourea linker is prepared by the reaction of an amine group on one component with an isocyanate or thioisocyanate group on the second component.

An amidine linker is prepared by the reaction of an amine group on one component with an imidoester group on the second component.

An amine linker is made by the alkylation reaction of an amine group on one component with an alkylating group such as halide, epoxide, or sulfonate ester group on the second component. Alternatively, an amine linker can also be made by reductive amination of an amine group on one component with an aldehyde or ketone group on the second component with a suitable reducing reagent such as sodium cyanoborohydride or sodium triacetoxyborohydride.

A sulfonamide linker is made by the reaction of an amine group on one component with a sulfonyl halide (such as sulfonyl chloride) group on the second component.

A sulfone linker is made by Michael addition of a nucleophile to a vinyl sulfone. Either the vinyl sulfone or the nucleophile may be on the surface of the nanocarrier or attached to a component.

The component can also be conjugated via non-covalent conjugation methods. For example, a negative charged immunosuppressant can be conjugated to a positive charged component through electrostatic adsorption. A component containing a metal ligand can also be conjugated to a metal complex via a metal-ligand complex.

In embodiments, the component can be attached to a polymer, for example polylactic acid-block-polyethylene glycol, prior to the assembly of a synthetic nanocarrier or the synthetic nanocarrier can be formed with reactive or activatible groups on its surface. In the latter case, the component may be prepared with a group which is compatible with the attachment chemistry that is presented by the synthetic nanocarriers' surface. In other embodiments, a peptide component can be attached to VLPs or liposomes using a suitable linker. A linker is a compound or reagent that capable of coupling two molecules together. In an embodiment, the linker can be a homobifuntional or heterobifunctional reagent as described in Hermanson 2008. For example, an VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a peptide component containing an acid group via the other end of the ADH linker on nanocarrier to produce the corresponding VLP or liposome peptide conjugate.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The component is prepared with the presence of either an alkyne (if the polymer contains an azide) or an azide (if the polymer contains an alkyne) group. The component is then allowed to react with the nanocarrier via the 1,3-dipolar cycloaddition reaction with or without a catalyst which covalently attaches the component to the particle through the 1,4-disubstituted 1,2,3-triazole linker.

If the component is a small molecule, it may be of advantage to attach the component to a polymer prior to the assembly of synthetic nanocarriers. In embodiments, it may also be an advantage to prepare the synthetic nanocarriers with surface groups that are used to attach the component to the synthetic nanocarrier through the use of these surface groups rather than attaching the component to a polymer and then using this polymer conjugate in the construction of synthetic nanocarriers.

For detailed descriptions of available conjugation methods, see Hermanson G T “Bioconjugate Techniques”, 2nd Edition Published by Academic Press, Inc., 2008. In addition to covalent attachment the component can be attached by adsorption to a pre-formed synthetic nanocarrier or it can be attached by encapsulation during the formation of the synthetic nanocarrier.

Synthetic nanocarriers may be prepared using a wide variety of methods known in the art. For example, synthetic nanocarriers can be formed by methods such as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

Materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. No. 6,632,671 to Unger issued Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be attached to the synthetic nanocarriers and/or the composition of the polymer matrix.

If synthetic nanocarriers prepared by any of the above methods have a size range outside of the desired range, synthetic nanocarriers can be sized, for example, using a sieve.

Elements of the synthetic nanocarriers may be attached to the overall synthetic nanocarrier, e.g., by one or more covalent bonds, or may be attached by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be attached to components directly or indirectly via non-covalent interactions. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such attachments may be arranged to be on an external surface or an internal surface of a synthetic nanocarrier. In embodiments, encapsulation and/or absorption is a form of attaching.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention may comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, compositions are suspended in sterile saline solution for injection with a preservative.

It is to be understood that the compositions of the invention can be made in any suitable manner, and the invention is in no way limited to compositions that can be produced using the methods described herein. Selection of an appropriate method of manufacture may require attention to the properties of the particular moieties being associated.

In some embodiments, compositions are manufactured under sterile conditions or are terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection.

Administration according to the present invention may be by a variety of routes, including but not limited to subcutaneous, intravenous, and intraperitoneal routes. The compositions referred to herein may be manufactured and prepared for administration, in some embodiments concomitant administration, using conventional methods.

The compositions of the invention can be administered in effective amounts, such as the effective amounts described elsewhere herein. In some embodiments, the synthetic nanocarriers comprising an immunosuppressant and/or viral vectors are present in dosage forms in an amount effective to reduce an immune response and/or allow for readministration of a viral vector to a subject. In some embodiments, the synthetic nanocarriers comprising an immunosuppressant and/or viral vectors are present in dosage forms in an amount effective to escalate or achieve efficacious transgene expression in a subject. Dosage forms may be administered at a variety of frequencies. In some embodiments, repeated administration of synthetic nanocarriers comprising an immunosuppressant with a viral vector is undertaken.

Aspects of the invention relate to determining a protocol for the methods of administration as provided herein. A protocol can be determined by varying at least the frequency, dosage amount of the viral vector and synthetic nanocarriers comprising an immunosuppressant and subsequently assessing a desired or undesired immune response. A preferred protocol for practice of the invention reduces an immune response against the viral vector and/or the expressed product and/or promotes transgene expression. The protocol comprises at least the frequency of the administration and doses of the viral vector and synthetic nanocarriers comprising an immunosuppressant.

Another aspect of the disclosure relates to kits. In some embodiments, the kit comprises any one or more of the compositions provided herein. Preferably, the composition(s) is/are in an amount to provide any one or more doses as provided herein. The composition(s) can be in one container or in more than one container in the kit. In some embodiments of any one of the kits provided, the container is a vial or an ampoule. In some embodiments of any one of the kits provided, the composition(s) are in lyophilized form each in a separate container or in the same container, such that they may be reconstituted at a subsequent time. In some embodiments of any one of the kits provided, the kit further comprises instructions for reconstitution, mixing, administration, etc. In some embodiments of any one of the kits provided, the instructions include a description of any one of the methods described herein. Instructions can be in any suitable form, e.g., as a printed insert or a label. In some embodiments of any one of the kits provided herein, the kit further comprises one or more syringes or other device(s) that can deliver the composition(s) in vivo to a subject.

### EXAMPLES

### Example 1: ssAAV Vector Construct Experiments In Vitro

A series of ssAAV vector constructs expressing the human OTC transgene under the transcriptional control of a liver-specific promoter were developed. The rAAV-hOTC vector (AAV2/8, i.e., an AAV2 virus engineered to have AAV8 capsid proteins) contains a human OTC (hOTC) expression cassette flanked by wild-type AAV2 inverted terminal repeats (ITRs). The backbone, promoter, and regulatory elements are based on the vector pSMD2 (Ronzitti, et al., 2016). Transcription of the hOTC transgene is driven by a hybrid promoter containing apolipoprotein E (ApoE) enhancer and human alpha-1-antitrypsin (hAAT) promoter and terminated by the hemoglobin beta (HBB) polyadenylation signal. The coding region and the promoter are separated by a human hemoglobin beta-derived synthetic intron (HBB2) that was modified by removal of alternative open reading frames longer than 50 base pairs and cryptic splicing sites (Ronzitti, et al., 2016).

The wt-hOTC was codon-optimized (CO) with different algorithms. The optimization process is aimed at improving translation and stability of the OTC mRNA by changing the nucleotide sequence while keeping the amino acid primary sequence unvaried. The wild-type OTC cDNA sequence and the codon-optimized (CO) LW4 sequence from WO 2015/138357 patent A2 (Wang L., Wilson J. M.) were also synthesized, to be used as comparison control (CO1). The nucleotide sequence of the different CO cDNAs differs with a range of 30-20% from the WT cDNA sequence. The vectors were then packaged into the AAV8 serotype and used to transduce Huh7 cells. Huh7 cells, co-transfected with the OTC constructs and the pGG2-eGFP plasmid (to normalize for transfection efficiency), were used to generate total RNA and proteins. mRNA, protein, and activity levels were analyzed using qRT-PCR and Western blotting.

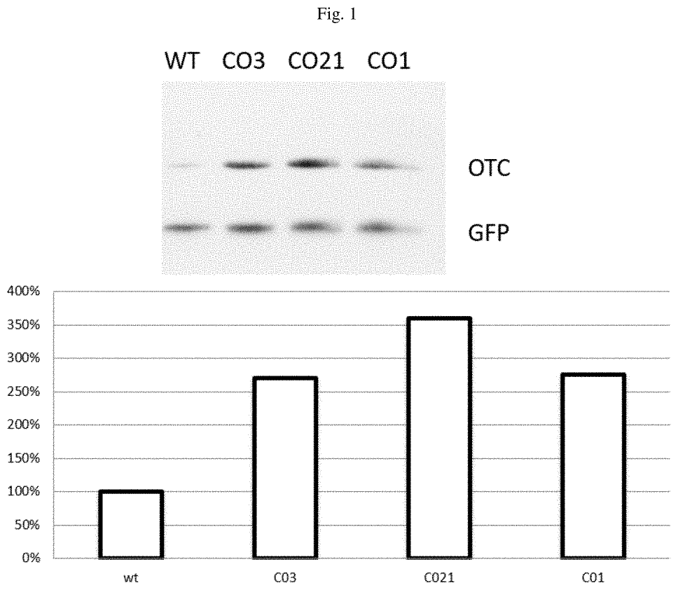

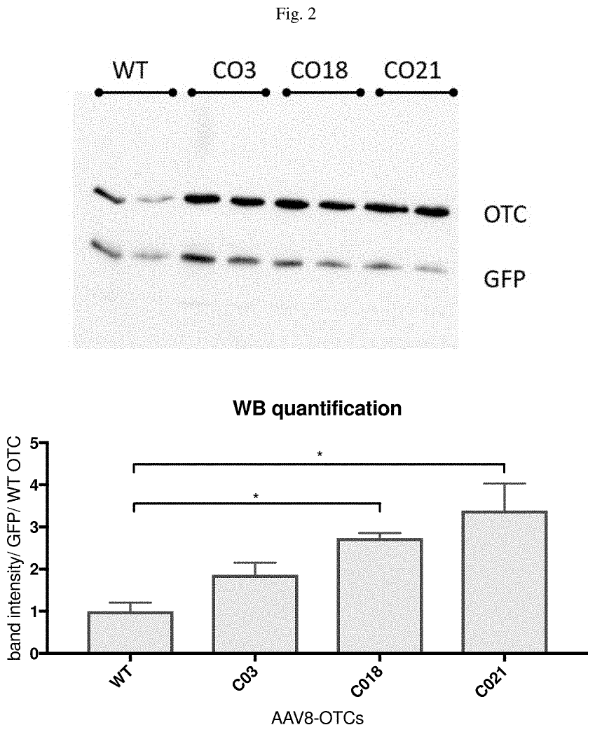

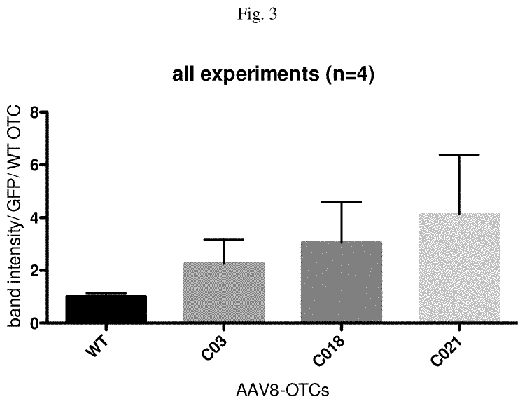

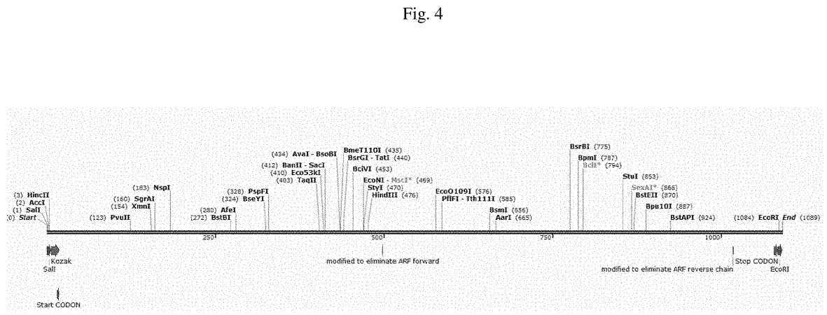

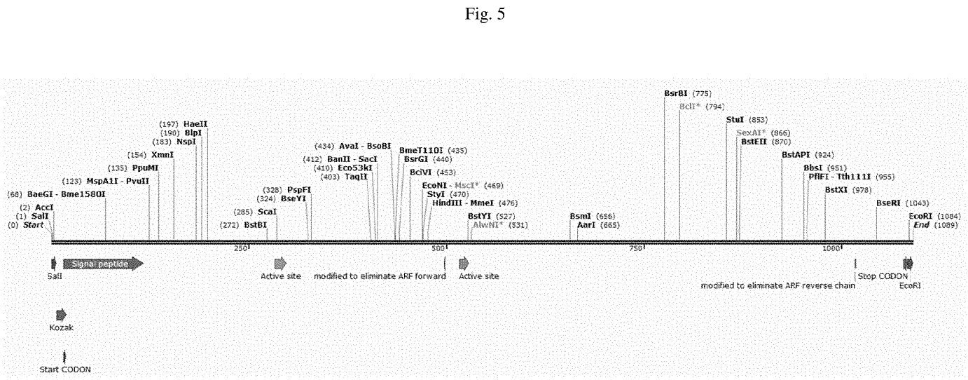

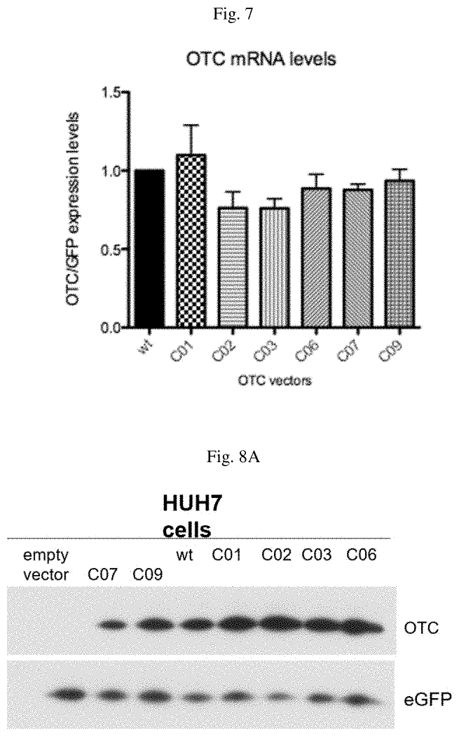

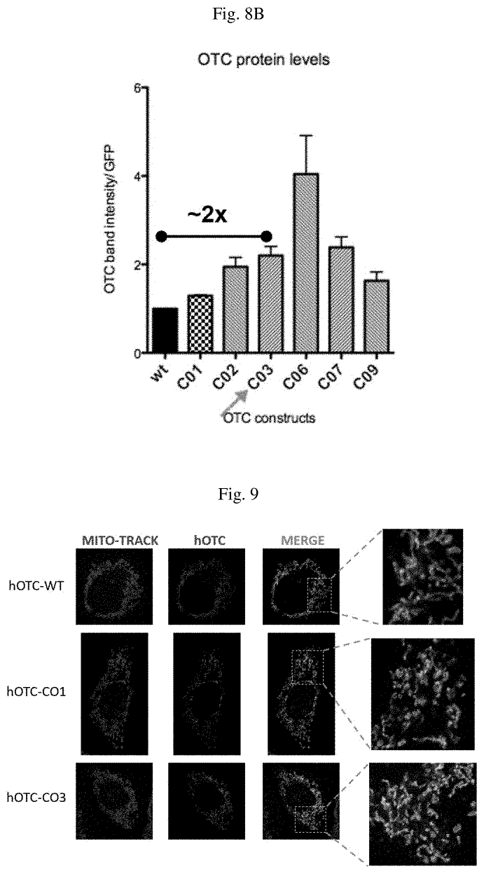

Transfection was demonstrated using a GFP plasmid to normalize for efficiency. The resulting DNA was amplified (top, FIG. 1) and the CO3 and CO21 constructs showed the greatest transfection efficiency (bottom, FIG. 1). Features of the CO3 and CO21 constructs are shown in FIGS. 4 and 5, respectively. When the constructs were run in duplicate and then quantified, significant differences were seen between the wild-type (GFP plasmid) and the CO18 and CO21 vectors (FIG. 2). The results from all of the experiments (n=4) were averaged and are presented in FIG. 3. CO3 was examined in the same manner; the results are shown in FIGS. 7-8B.

Treated cells were also stained to examine the subcellular localization of the OTC (FIG. 9).

All DNA preparations were done in parallel on the same day with the same DNA prep kit in order to avoid major differences in DNA quality and quantification.

### Example 2: ssAAV Vector Construct Experiments in Mice

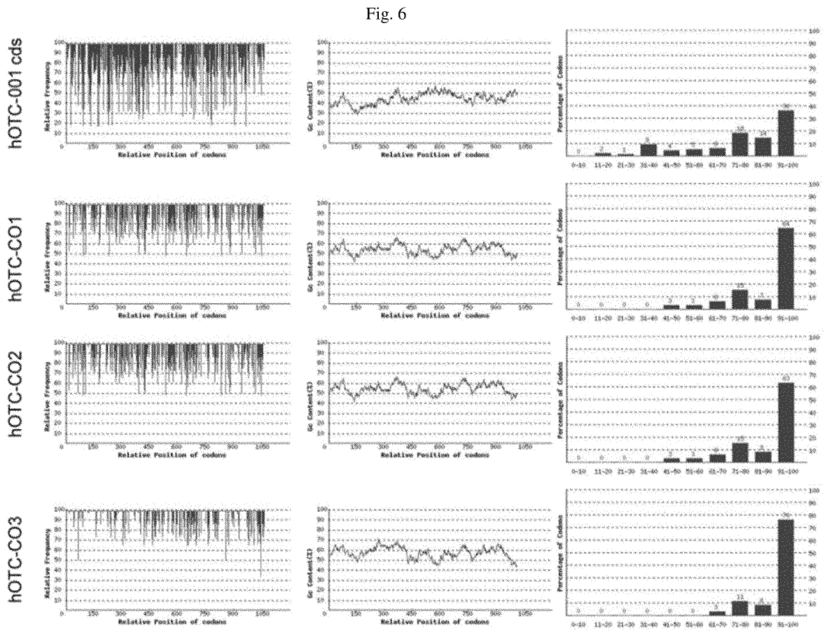

A series of ssAAV vector constructs expressing the human OTC transgene under the transcriptional control of a liver-specific promoter were developed. The wt-hOTC was codon-optimized (CO) with different algorithms (FIG. 6, Table 1). The different algorithms, including codon usage, cryptic splicing sites, ORFs in the antisense strand (ARF >50 bp), secondary structure, GC-content, and CpG islands, were examined and then manual analysis was conducted to determine candidate constructs. The vectors were then packaged into the AAV8 serotype and used to transduce male and female WT C57Bl/6 and OTC^{spf-ash} mice.

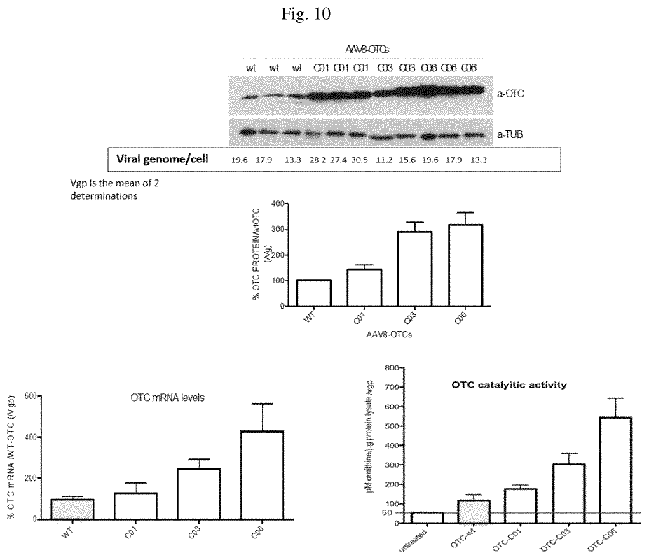

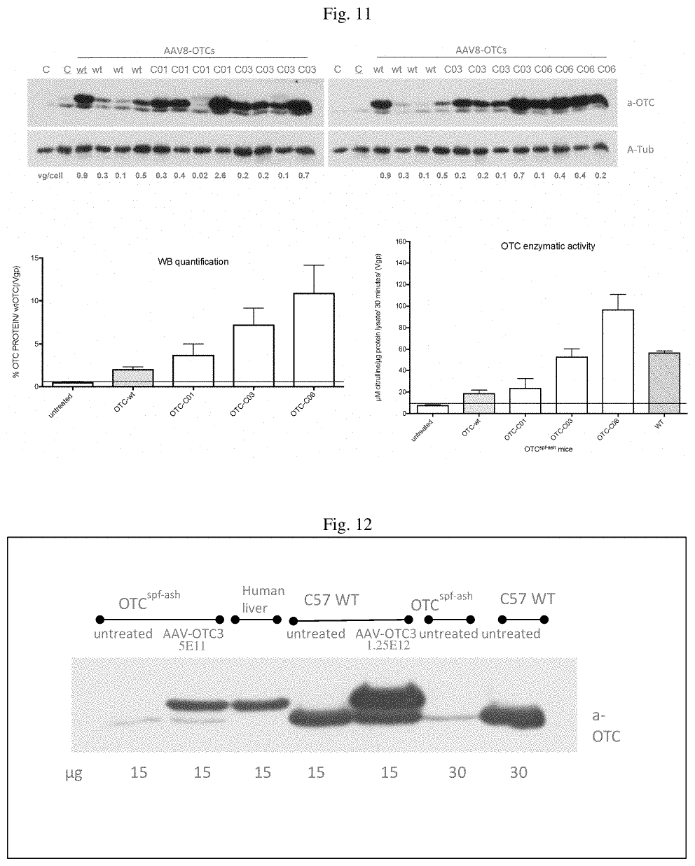

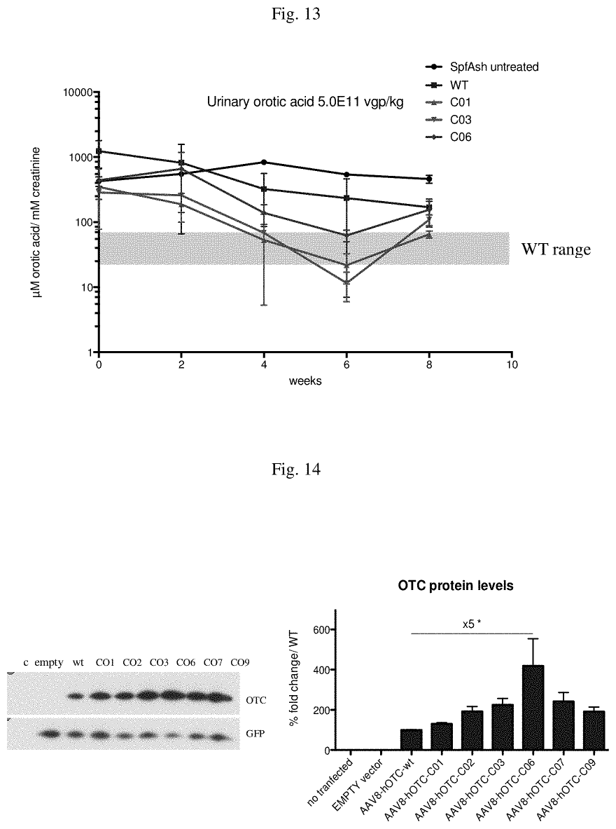

Additionally, protein levels, catalytic activity (FIGS. 10-11), and urinary orotic acid levels (FIG. 13) were measured, leading to the identification of a CO-hOTC construct that was particularly efficient in correcting the phenotype of OTC^{spf-ash} mice (CO3). Protein, activity and mRNA quantification were normalized by viral genomes.

TABLE 1
Exemplary Transgene Sequences

| | | SEQ |
|---|---|---|
| | | ID |
| Name | Sequence | NO: |
| hOCT- | GTCGACgccgccaccATGCTGTTTAATCTGAGGATCCTGTTAAACAATGCAGCTTTTAG | 1 |
| 001_cds_(WT) | AAATGGTCACAACTTCATGGTTCGAAATTTTCGGTGTGGACAACCACTACAAAATAA | |
| | AGTGCAGCTGAAGGGCCGTGACCTTCTCACTCTAAAAAACTTTACCGGAGAAGAAA | |
| | TTAAATATATGCTATGGCTATCAGCAGATCTGAAATTTAGGATAAAACAGAAAGGAG | |
| | AGTATTTGCCTTTATTGCAAGGGAAGTCCTTAGGCATGATTTTTGAGAAAAGAAGTA | |
| | CTCGAACAAGATTGTCTACAGAAACAGGCTTTGCACTTCTGGGAGGACATCCTTGTT | |
| | TTCTTACCACACAAGATATTCATTTGGGTGTGAATGAAAGTCTCACGGACACGGCCC | |
| | GTGTATTGTCTAGCATGGCAGATGCAGTATTGGCTCGAGTGTATAAACAATCAGATT | |
| | TGGACACCCTGGCTAAAGAAGCATCCATCCCAATTATCAATGGGCTGTCAGATTTGT | |
| | ACCATCCTATCCAGATCCTGGCTGATTACCTCACGCTCCAGGAACACTATAGCTCTC | |
| | TGAAAGGTCTTACCCTCAGCTGGATCGGGGATGGGAACAATATCCTGCACTCCATC | |
| | ATGATGAGCGCAGCGAAATTCGGAATGCACCTTCAGGCAGCTACTCCAAAGGGTTA | |
| | TGAGCCGGATGCTAGTGTAACCAAGTTGGCAGAGCAGTATGCCAAAGAGAATGGTA | |
| | CCAAGCTGTTGCTGACAAATGATCCATTGGAAGCAGCGCATGGAGGCAATGTATTA | |
| | ATTACAGACACTTGGATAAGCATGGGACAAGAAGAGGAGAAGAAAAAGCGGCTCCA | |
| | GGCTTTCCAAGGTTACCAGGTTACAATGAAGACTGCTAAAGTTGCTGCCTCTGACT | |
| | GGACATTTTTACACTGCTTGCCCAGAAAGCCAGAAGAAGTGGATGATGAAGTCTTTT | |
| | ATTCTCCTCGATCACTAGTGTTCCCAGAGGCAGAAAACAGAAAGTGGACAATCATG | |
| | GCTGTCATGGTGTCCCTGCTGACAGATTACTCACCTCAGCTCCAGAAGCCTAAATTT | |
| | TGATAAgaattc | |
| hOCT-CO1 | GTCGACgccgccaccATGCTGTTCAACCTGCGAATCCTGCTGAACAACGCCGCTTTTC | 2 |
| | GGAACGGGCACAACTTTATGGTGAGGAACTTTCGCTGCGGACAGCCCCTCCAGAA | |
| | TAAGGTCCAGCTGAAGGGCAGGGACCTGCTGACCCTGAAAAATTTCACAGGGGAG | |
| | GAAATCAAGTATATGCTGTGGCTGTCAGCTGATCTGAAGTTCCGGATCAAGCAGAA | |
| | GGGCGAATATCTGCCTCTGCTCCAGGGCAAAAGCCTGGGGATGATCTTCGAAAAG | |
| | CGCAGTACTCGGACCAGACTGTCAACCGAGACTGGATTCGCTCTGCTGGGAGGAC | |
| | ACCCTTGTTTTCTGACCACTCAGGACATTCACCTGGGAGTGAACGAGTCCCTGACC | |
| | GACACTGCTCGCGTCCTGAGCTCTATGGCCGACGCTGTGCTGGCTCGAGTCTACA | |
| | AACAGTCCGACCTGGATACCCTGGCCAAGGAAGCTTCTATCCCAATTATTAACGGC | |
| | CTGTCAGACCTGTATCACCCCATCCAGATTCTGGCCGATTACCTGACCCTCCAGGA | |
| | GCACTATTCTAGTCTGAAAGGGCTGACACTGAGTTGGATTGGGGACGGAAACAATA | |
| | TCCTGCACTCTATTATGATGTCAGCCGCCAAGTTTGGAATGCACCTCCAGGCTGCA | |
| | ACCCCAAAAGGCTACGAACCCGATGCCTCAGTGACAAAGCTGGCTGAACAGTACG | |
| | CCAAAGAGAACGGCACTAAGCTGCTGCTGACCAACGACCCTCTGGAGGCCGCTCA | |
| | CGGAGGCAACGTGCTGATCACCGATACCTGGATTAGTATGGGACAGGAGGAAGAG | |
| | AAGAAGAAGCGGCTCCAGGCCTTCCAGGGCTACCAGGTGACAATGAAAACCGCTA | |
| | AGGTCGCAGCCAGCGATTGGACCTTTCTGCACTGCCTGCCCAGAAAGCCCGAAGA | |
| | GGTGGACGACGAGGTCTTCTACTCTCCCAGAAGCCTGGTGTTTCCCGAAGCTGAG | |
| | AATAGGAAGTGGACAATTATGGCAGTGATGGTCAGCCTGCTGACTGATTATTCACC | |
| | TCAGCTCCAGAAACCAAAGTTCTGATAAgaattc | |
| hOCT-CO2 | GTCGACgccgccaccATGCTGTTCAACCTGCGAATCCTGCTGAACAACGCCGCTTTTC | 3 |
| | GGAACGGGCACAACTTTATGGTCCGCAACTTTCGCTGCGGACAGCCCCTCCAGAAT | |
| | AAGGTCCAGCTGAAGGGCAGGGACCTGCTGACCCTGAAAAATTTCACAGGGGAGG | |
| | AAATCAAGTATATGCTGTGGCTGTCAGCTGATCTGAAGTTCCGGATCAAGCAGAAG | |
| | GGCGAATATCTGCCACTGCTGCAGGGCAAAAGCCTGGGGATGATCTTCGAAAAGC | |
| | GCAGTACTCGGACCAGACTGTCAACCGAGACTGGATTCGCTCTGCTGGGAGGACA | |
| | CCCTTGTTTTCTGACAACTCAGGACATTCACCTGGGAGTGAACGAGTCCCTGACCG | |
| | ACACTGCTCGCGTCCTGAGCTCTATGGCCGACGCTGTGCTGGCTCGAGTCTACAAA | |
| | CAGTCCGACCTGGATACCCTGGCCAAGGAAGCTTCTATCCCAATTATTAACGGCCT | |
| | GTCAGACCTGTATCACCCCATCCAGATTCTGGCCGATTACCTGACCCTCCAGGAGC | |
| | ACTATTCTAGTCTGAAAGGGCTGACACTGAGTTGGATTGGGGACGGAAACAATATC | |
| | CTGCACTCTATTATGATGTCAGCCGCCAAGTTTGGAATGCACCTCCAGGCTGCAAC | |
| | CCCAAAAGGCTACGAACCCGATGCCTCAGTGACAAAGCTGGCTGAACAGTACGCC | |
| | AAAGAGAACGGCACTAAGCTGCTGCTGACCAACGACCCTCTGGAGGCCGCTCACG | |
| | GAGGCAACGTGCTGATCACCGATACCTGGATTAGTATGGGACAGGAGGAAGAGAA | |
| | GAAGAAGCGGCTCCAGGCCTTCCAGGGCTACCAGGTCACCATGAAAACCGCTAAG | |
| | GTCGCAGCCAGCGATTGGACCTTTCTGCACTGCCTGCCCAGAAAGCCCGAAGAGG | |
| | TGGACGACGAGGTCTTCTACTCTCCACGCTCCCTGGTGTTTCCCGAAGCTGAGAAT | |
| | AGGAAGTGGACAATTATGGCAGTGATGGTCAGCCTGCTGACTGATTATTCACCTCA | |
| | GCTCCAGAAACCAAAGTTCTGATAAgaattc | |
| hOCT-CO3 | GTCGACgccgccaccATGCTGTTCAACCTGAGAATCCTGCTGAACAACGCCGCCTTTC | 4 |
| | GGAACGGCCACAACTTCATGGTCCGCAACTTCCGCTGCGGCCAGCCACTGCAGAA | |
| | CAAGGTGCAGCTGAAGGGCAGAGACCTGCTGACCCTGAAGAACTTCACCGGCGAG | |
| | GAGATCAAATACATGCTGTGGCTGAGCGCCGATCTGAAGTTCAGAATCAAGCAGAA | |
| | GGGCGAGTACCTGCCACTGCTGCAGGGCAAAAGCCTGGGCATGATCTTCGAAAAG | |
| | CGCTCCACCCGGACCAGACTGAGCACCGAGACCGGCTTCGCTCTGCTGGGAGGC | |
| | CACCCTTGCTTCCTGACAACCCAGGACATCCACCTGGGCGTGAACGAGTCCCTGA | |
| | CCGACACCGCCAGAGTGCTGAGCTCTATGGCCGACGCCGTGCTGGCTCGGGTGTA | |
| | CAAACAGTCCGACCTGGATACCCTGGCCAAGGAAGCTTCCATCCCAATTATTAAcG | |
| | GCCTGTCCGACCTGTATCACCCCATCCAGATTCTGGCCGATTACCTGACCCTGCAG | |
| | GAGCACTATAGCAGCCTGAAGGGCCTGACACTGTCTTGGATCGGCGACGGAAATA | |
| | ATATCCTGCACTCTATTATGATGTCTGCCGCCAAGTTTGGAATGCACCTGCAGGCT | |
| | GCTACCCCTAAAGGCTACGAACCCGATGCCTCTGTGACAAAGCTGGCTGAACAGTA | |
| | CGCCAAAGAGAACGGCACAAAGCTGCTGCTGACCAACGACCCTCTGGAGGCCGCT | |
| | CACGGAGGCAACGTGCTGATCACCGATACCTGGATTTCTATGGGACAGGAGGAAG | |
| | AGAAGAAGAAGCGGCTGCAGGCCTTCCAGGGCTACCAGGTCACCATGAAAACCGC | |
| | TAAGGTGGCCGCCAGCGATTGGACCTTTCTGCACTGCCTGCCCAGAAAGCCCGAA | |
| | GAGGTGGACGACGAGGTGTTCTACAGCCCCCGGAGCCTGGTGTTTCCCGAAGCTG | |
| | AGAATCGGAAATGGACAATtATGGCTGTGATGGTGTCCCTGCTGACTGATTATTCTC | |
| | CTCAACTGCAGAAACCTAAATTTTGATAAgaattc | |
| HOCT-CO6 | GTCGACgccgccaccATGCTGTTCAACCTGCGCATCCTGCTGAACAACGCCGCCTTCC | 5 |
| | GCAACGGCCACAACTTCATGGTcagaAACTTCCGCTGCGGCCAGCCCCTGCAaAACA | |
| | AaGTGCAGCTGAAGGGCCGCGACCTGCTGACCCTGAAGAACTTCACCGGCGAGGA | |
| | GATCAAaTACATGCTGTGGCTGAGCGCCGACCTGAAGTTCCGCATCAAGCAGAAGG | |
| | GCGAGTACCTGCCaCTGCTGCAaGGCAAGAGCCTGGGCATGATCTTCGAGAAGCG | |
| | CAGCACCCGCACCCGCCTGAGCACCGAGACCGGCTTCGCCCTGCTGGGCGGCCA | |
| | CCCCTGCTTCCTGACaACaCAGGACATCCACCTGGGCGTGAACGAGAGCCTGACC | |
| | GACACCGCCCGCGTGCTGAGCAGCATGGCCGACGCCGTGCTGGCCCGCGTGTAC | |
| | AAGCAGAGCGACCTGGACACCCTGGCCAAGGAGGCCAGCATCCCCATCATCAACG | |
| | GCCTGAGCGACCTGTACCACCCCATCCAGATCCTGGCCGACTACCTGACCCTGCA | |
| | GGAGCACTACAGCAGCCTGAAGGGCCTGACCCTGAGCTGGATCGGCGACGGCAA | |
| | CAAtATCCTGCACAGtATtATGATGAGCGCCGCCAAGTTCGGaATGCACCTGCAGGC | |
| | CGCCACCCCCAAGGGCTACGAGCCCGACGCCAGCGTGACCAAGCTGGCCGAGCA | |
| | GTACGCCAAGGAGAACGGCACCAAGCTGCTGCTGACCAACGACCCCCTGGAGGCC | |
| | GCCCACGGCGGCAACGTGCTGATCACCGACACCTGGATCtctATGGGCCAGGAGGA | |
| | GGAGAAGAAGAAGCGCCTGCAGGCCTTCCAGGGCTACCAGGTcACtATGAAGACCG | |
| | CCAAGGTGGCCGCCAGCGACTGGACCTTCCTGCACTGCCTGCCCCGCAAGCCCGA | |
| | GGAGGTGGACGACGAGGTGTTCTACAGCCCCCGCAGCCTGGTGTTCCCCGAGGC | |
| | CGAGAACCGCAAGTGGACtATtATGGCCGTGATGGTctccCTGCTGACCGACTACAGC | |
| | CCCCAGCTGCAGAAGCCCAAGTTCTGATAAgaattc | |
| HOTC-CO6-1 | GTCGACgccgccaccATGCTGTTCAACCTGCGGATCCTGCTGAACAACGCCGCCTTCA | 6 |
| | GAAACGGCCACAACTTCATGGTCCGAAACTTCAGATGCGGCCAGCCTCTGCAGAAC | |
| | AAGGTGCAGCTGAAGGGCAGAGATCTGCTGACCCTGAAGAACTTCACCGGCGAAG | |
| | AGATCAAATACATGCTGTGGCTGAGCGCCGACCTGAAGTTCCGGATTAAGCAGAAG | |
| | GGCGAGTACCTGCCACTGCTGCAGGGAAAGTCTCTGGGCATGATCTTCGAGAAGC | |
| | GGAGCACCAGAACCAGACTGAGCACCGAGACAGGCTTTGCCCTGCTCGGAGGACA | |
| | CCCCTGCTTTCTGACAACACAGGATATCCACCTGGGCGTGAACGAGAGCCTGACC | |
| | GATACAGCCAGAGTGCTGAGCAGCATGGCTGATGCCGTGCTGGCCAGAGTGTACA | |
| | AGCAGAGCGATCTGGACACCCTGGCCAAAGAGGCCAGCATTCCCATCATCAACGG | |
| | CCTGAGCGACCTGTATCACCCCATCCAGATCCTGGCCGACTACCTGACACTGCAAG | |
| | AGCACTACAGCAGCCTGAAGGGACTGACCCTGTCTTGGATCGGCGACGGCAACAA | |
| | CATCCTGCACTCTATTATGATGAGCGCCGCCAAGTTCGGAATGCACCTGCAGGCCG | |
| | CTACACCCAAGGGCTATGAGCCTGATGCCAGCGTGACAAAGCTGGCCGAGCAGTA | |
| | CGCCAAAGAGAACGGCACAAAGCTGCTGCTGACCAACGATCCCCTGGAAGCTGCC | |
| | CACGGCGGCAATGTGCTGATCACCGATACCTGGATCTCTATGGGCCAAGAGGAAG | |
| | AGAAGAAGAAGCGGCTGCAGGCCTTCCAGGGCTACCAAGTGACAATGAAGACCGC | |
| | CAAGGTGGCCGCCAGCGATTGGACCTTTCTGCACTGCCTGCCTCGGAAGCCTGAA | |
| | GAGGTGGACGACGAGGTGTTCTACAGCCCTAGAAGCCTGGTGTTCCCCGAGGCCG | |
| | AGAACAGAAAGTGGACCATCATGGCTGTGATGGTGTCCCTGCTGACCGACTACTCT | |
| | CCCCAGCTGCAGAAGCCCAAGTTCTGATAAgaattc | |
| hOCT-CO7 | GTCGACgccgccaccATGCTGTTCAACCTGCGGATCCTGCTGAACAACGCCGCCTTCC | 7 |
| | GGAACGGCCACAACTTCATGGTCCGGAACTTCCGCTGTGGCCAGCCCCTGCAGAA | |
| | CAAGGTGCAGCTGAAGGGCAGGGACCTGCTGACCCTGAAGAACTTCACCGGCGAA | |
| | GAGATCAAATACATGCTGTGGCTGAGCGCCGACCTGAAGTTCCGGATCAAGCAGAA | |
| | GGGCGAGTACCTGCCACTGCTGCAGGGCAAGTCTCTGGGCATGATCTTCGAGAAG | |
| | CGGAGCACCCGGACCCGGCTGTCTACCGAGACAGGATTTGCCCTGCTGGGCGGC | |
| | CACCCTTGCTTTCTGACAACACAGGATATCCACCTGGGCGTGAACGAGAGCCTGAC | |
| | CGACACAGCCAGAGTGCTGAGCAGCATGGCCGACGCCGTGCTGGCCAGAGTGTA | |
| | CAAGCAGAGCGACCTGGACACCCTGGCCAAAGAGGCCAGCATCCCCATCATCAAC | |
| | GGCCTGTCCGACCTGTACCACCCCATCCAGATCCTGGCAGACTACCTCACACTGCA | |
| | GGAACACTACAGCTCCCTGAAGGGCCTGACACTGAGCTGGATCGGCGACGGCAAC | |
| | AATATCCTGCACTCTATTATGATGAGCGCCGCCAAGTTCGGAATGCACCTGCAGGC | |
| | CGCCACCCCCAAGGGCTACGAGCCTGACGCCAGCGTGACCAAGCTGGCCGAGCA | |
| | GTACGCCAAAGAGAACGGCACCAAGCTGCTGCTGACCAACGACCCTCTGGAAGCC | |
| | GCCCACGGCGGCAACGTGCTGATCACCGATACCTGGATCTCTATGGGCCAGGAAG | |
| | AGGAAAAGAAGAAGCGGCTGCAGGCCTTCCAGGGCTACCAAGTGACAATGAAGAC | |
| | CGCCAAAGTGGCCGCCAGCGACTGGACCTTCCTGCACTGCCTGCCCAGAAAGCCC | |
| | GAAGAGGTGGACGACGAGGTGTTCTACAGCCCCCGGTCCCTGGTGTTTCCCGAGG | |
| | CCGAGAACCGGAAGTGGACAATTATGGCTGTGATGGTGTCTCTGCTGACCGACTAC | |
| | TCCCCCCAGCTGCAGAAGCCCAAGTTCTGATAAgaattc | |
| hOTC-CO9 | GTCGACgccgccaccATGCTCTTTAATCTGAGAATCCTGCTTAACAACGCCGCCTTCCG | 8 |
| | CAACGGACACAACTTCATGGTCCGGAACTTCAGATGCGGCCAGCCTCTCCAAAACA | |
| | AGGTCCAGCTTAAGGGCAGAGATCTGCTCACCCTCAAAAACTTCACCGGAGAGGAA | |
| | ATCAAGTATATGCTGTGGCTGTCTGCCGATCTTAAGTTCCGGATCAAACAGAAGGG | |
| | GGAGTACCTTCCGCTGCTGCAAGGGAAGTCACTCGGAATGATCTTCGAGAAGCGC | |
| | TCCACTCGGACCAGGCTCAGCACCGAAACTGGATTTGCACTCCTGGGTGGTCATCC | |
| | CTGTTTCCTGACCACCCAAGATATCCACCTGGGCGTGAACGAATCCCTGACCGACA | |
| | CAGCTCGCGTGCTGTCCTCCATGGCCGACGCTGTGTTGGCCCGGGTCTACAAGCA | |
| | GAGCGACCTGGACACTCTGGCCAAGGAAGCCTCCATTCCGATCATCAATGGGCTG | |
| | TCCGACCTGTACCACCCAATTCAGATCCTGGCGGATTACTTGACCCTGCAAGAGCA | |
| | CTACAGCTCCCTGAAGGGACTGACCCTCTCCTGGATTGGCGACGGGAACAACATC | |
| | CTCCACTCGATTATGATGTCGGCGGCGAAGTTCGGCATGCATCTGCAAGCCGCCA | |
| | CTCCTAAGGGTTACGAACCGGACGCAAGCGTGACCAAGCTCGCCGAACAGTACGC | |
| | GAAGGAAAACGGCACTAAGCTGCTGCTGACCAACGACCCCCTGGAAGCCGCTCAC | |
| | GGCGGAAACGTGCTGATTACGGACACCTGGATCAGCATGGGACAGGAGGAGGAGA | |
| | AGAAGAAGCGGCTGCAGGCGTTCCAGGGATACCAGGTCACCATGAAAACTGCCAA | |
| | AGTGGCAGCCTCAGACTGGACTTTCCTGCACTGCCTGCCTCGGAAGCCAGAGGAG | |
| | GTGGACGATGAAGTGTTCTACTCCCCTCGCTCCCTGGTGTTCCCGGAGGCGGAAA | |
| | ACAGGAAGTGGACCATCATGGCCGTGATGGTGTCATTGTTGACCGATTACTCGCCG | |
| | CAACTGCAGAAGCCCAAGTTTTGAgaattc | |
| hOTC-CO9-1 | GTCGACgccgccaccATGCTGTTTAATCTGAGAATCCTGCTGAACAATGCTGCTTTCCG | 9 |
| | CAATGGGCACAACTTTATGGTCCGAAACTTCCGATGTGGACAGCCTCTGCAGAACA | |
| | AGGTGCAGCTGAAGGGCCGGGACCTGCTGACCCTGAAGAATTTCACAGGCGAGGA | |
| | GATCAAATACATGCTGTGGCTGAGCGCCGATCTGAAGTTTAGGATCAAGCAGAAGG | |
| | GCGAGTATCTGCCACTGCTGCAGGGCAAGTCCCTGGGCATGATCTTCGAGAAGCG | |
| | GAGCACCCGGACAAGACTGAGCACCGAGACAGGATTCGCACTGCTGGGAGGACAC | |
| | CCATGCTTTCTGACAACACAGGACATCCACCTGGGCGTGAACGAGTCTCTGACCGA | |
| | CACAGCACGGGTGCTGAGCTCCATGGCAGATGCCGTGCTGGCCAGAGTGTACAAG | |
| | CAGAGCGACCTGGATACCCTGGCCAAGGAGGCCTCCATCCCCATCATCAATGGCC | |
| | TGTCTGACCTGTATCACCCAATCCAGATCCTGGCCGATTACCTGACCCTGCAGGAG | |
| | CACTATTCTAGCCTGAAGGGCCTGACACTGAGCTGGATCGGCGACGGCAACAATAT | |
| | CCTGCACAGCATCATGATGTCCGCCGCCAAGTTTGGAATGCACCTGCAGGCAGCA | |
| | ACCCCAAAGGGATACGAGCCCGATGCCTCCGTGACAAAGCTGGCCGAGCAGTATG | |
| | CCAAGGAGAACGGCACCAAGCTGCTGCTGACAAATGACCCACTGGAGGCAGCACA | |
| | CGGAGGAAACGTGCTGATCACCGATACATGGATCTCTATGGGCCAGGAGGAGGAG | |
| | AAGAAGAAGCGGCTGCAGGCCTTCCAGGGCTACCAGGTGACCATGAAGACAGCCA | |
| | AGGTGGCCGCCTCTGATTGGACCTTTCTGCACTGTCTGCCCCGGAAGCCTGAGGA | |
| | GGTGGACGATGAGGTGTTCTATTCCCCTCGGAGCCTGGTGTTCCCAGAGGCAGAG | |
| | AATCGCAAGTGGACAATCATGGCCGTGATGGTCTCCCTGCTGACTGACTACTCCCC | |
| | ACAGCTGCAGAAGCCCAAGTTTTGATAAgaattc | |
| hOTC-CO9-2 | GTCGACgccgccaccATGCTCTTCAATCTCCGAATACTCCTGAATAATGCGGCATTCAG | 10 |
| | AAATGGGCACAATTTTATGGTGCGAAATTTTCGATGTGGGCAGCCCTTGCAAAATAA | |
| | AGTACAACTTAAAGGCAGAGATCTTCTTACGCTCAAAAACTTTACCGGCGAAGAGAT | |
| | TAAGTATATGTTGTGGCTGTCTGCTGACCTTAAATTCCGAATAAAACAGAAAGGCGA | |
| | ATACCTTCCGCTCCTCCAAGGGAAATCTCTTGGGATGATTTTCGAGAAGAGATCTAC | |
| | GCGCACGCGGCTTTCAACGGAAACTGGATTCGCACTCTTGGGCGGCCACCCATGT | |
| | TTTCTGACTACTCAAGATATTCACTTGGGAGTAAATGAGTCACTTACTGACACGGCA | |
| | AGAGTGCTCTCTAGCATGGCTGATGCAGTGCTGGCTAGAGTCTATAAACAATCCGA | |
| | CCTGGATACACTCGCCAAGGAAGCTTCAATACCAATAATCAACGGGTTGTCTGACTT | |
| | GTACCACCCTATCCAAATCTTGGCCGATTATTTGACACTCCAGGAACACTACTCAAG | |
| | TCTGAAGGGACTTACGCTTAGCTGGATAGGTGATGGTAACAACATCCTTCATAGCAT | |
| | TATGATGTCAGCCGCCAAATTCGGCATGCATCTGCAAGCAGCGACTCCCAAGGGCT | |
| | ATGAGCCTGATGCCTCAGTCACCAAACTGGCGGAGCAGTACGCTAAAGAGAATGG | |
| | GACGAAGCTTTTGCTGACGAACGATCCCCTGGAGGCGGCTCACGGGGGAAATGTG | |
| | CTTATCACGGATACCTGGATAAGTATGGGGCAGGAGGAAGAAAAAAAAAAGCGATT | |
| | GCAAGCCTTTCAAGGTTACCAGGTTACAATGAAAACTGCGAAAGTCGCCGCATCTG | |
| | ACTGGACTTTTCTGCACTGTCTTCCGAGAAAGCCGGAAGAGGTGGACGACGAAGT | |
| | GTTCTACTCTCCGCGCTCTCTCGTGTTTCCTGAAGCAGAGAACCGAAAGTGGACCA | |
| | TAATGGCGGTAATGGTCAGCCTCTTGACTGATTATTCCCCTCAGCTGCAGAAGCCA | |
| | AAGTTTTGATAAgaattc | |
| hOTC-CO18 | GTCGACgccgccaccATGCTGTTCAACCTGCGCATCCTGCTGAACAACGCCGCCTTCC | 11 |
| | GCAACGGCCACAACTTCATGGTCAGAAACTTCCGCTGCGGCCAGCCCCTGCAAAA | |
| | CAAAGTGCAGCTGAAGGGCCGCGACCTGCTGACCCTGAAGAACTTCACCGGCGAG | |
| | GAGATCAAATACATGCTGTGGCTGAGCGCCGATCTGAAGTTCAGAATCAAGCAGAA | |
| | GGGCGAGTACCTGCCACTGCTGCAAGGCAAGAGCCTGGGCATGATCTTCGAGAAG | |
| | CGCAGCACCCGCACCCGCCTGAGCACCGAGACCGGCTTCGCTCTGCTGGGAGGC | |
| | CACCCTTGCTTCCTGACAACCCAGGACATCCACCTGGGCGTGAACGAGAGCCTGA | |
| | CCGACACCGCCCGCGTGCTGAGCAGCATGGCCGACGCCGTGCTGGCTCGGGTGT | |
| | ACAAACAGTCCGACCTGGATACCCTGGCCAAGGAAGCTTCCATCCCCATCATCAAC | |
| | GGCCTGAGCGACCTGTACCACCCCATCCAGATCCTGGCCGACTACCTGACCCTGC | |
| | AGGAGCACTACAGCAGCCTGAAGGGCCTGACCCTGAGCTGGATCGGCGACGGCA | |
| | ACAATATCCTGCACTCTATTATGATGTCTGCCGCCAAGTTTGGAATGCACCTGCAGG | |
| | CTGCTACCCCTAAAGGCTACGAACCCGATGCCTCTGTGACAAAGCTGGCTGAACAG | |
| | TACGCCAAAGAGAACGGCACAAAGCTGCTGCTGACCAACGACCCTCTGGAGGCCG | |
| | CTCACGGAGGCAACGTGCTGATCACCGACACCTGGATCTCTATGGGCCAGGAGGA | |
| | AGAGAAGAAGAAGCGGCTGCAGGCCTTCCAGGGCTACCAGGTCACCATGAAAACC | |
| | GCTAAGGTGGCCGCCAGCGATTGGACCTTCCTGCACTGCCTGCCCCGCAAGCCCG | |
| | AGGAGGTGGACGACGAGGTGTTCTACAGCCCCCGCAGCCTGGTGTTCCCCGAGG | |
| | CCGAGAACCGCAAGTGGACTATTATGGCCGTGATGGTGTCCCTGCTGACTGATTAT | |
| | TCTCCTCAACTGCAGAAACCTAAATTTTGATAAgaattc | |
| hOTC | GTCGACgccgccaccATGCTGTTCAACCTGCGAATCCTGCTGAACAACGCCGCTTTTC | 12 |
| CO21 | GGAACGGGCACAACTTTATGGTGAGGAACTTTCGCTGCGGACAGCCCCTCCAGAA | |
| (whole | TAAGGTCCAGCTGAAGGGCAGGGACCTGCTGACCCTGAAAAATTTCACAGGGGAG | |
| sequence) | GAAATCAAATACATGCTGTGGCTGAGCGCCGATCTGAAGTTCAGAATCAAGCAGAA | |
| | GGGCGAGTACCTGCCTCTGCTCCAGGGCAAAAGCCTGGGGATGATCTTCGAAAAG | |
| | CGCAGTACTCGGACCAGACTGTCAACCGAGACTGGCTTCGCTCTGCTGGGAGGCC | |
| | ACCCTTGCTTCCTGACAACCCAGGACATTCACCTGGGAGTGAACGAGTCCCTGACC | |
| | GACACTGCTCGCGTCCTGAGCTCTATGGCCGACGCCGTGCTGGCTCGGGTGTACA | |
| | AACAGTCCGACCTGGATACCCTGGCCAAGGAAGCTTCCATCCCCATCATCAACGGC | |
| | CTGAGCGACCTGTACCACCCCATCCAGATCCTGGCCGACTACCTGACCCTGCAGG | |
| | AGCACTACAGCAGCCTGAAGGGCCTGACCCTGAGCTGGATCGGCGACGGCAACAA | |
| | TATCCTGCACTCTATTATGATGTCTGCCGCCAAGTTTGGAATGCACCTGCAGGCTG | |
| | CTACCCCTAAAGGCTACGAACCCGATGCCTCTGTGACAAAGCTGGCTGAACAGTAC | |
| | GCCAAAGAGAACGGCACAAAGCTGCTGCTGACCAACGACCCTCTGGAGGCCGCTC | |
| | ACGGAGGCAACGTGCTGATCACCGATACCTGGATTAGTATGGGACAGGAGGAAGA | |
| | GAAGAAGAAGCGGCTGCAGGCCTTCCAGGGCTACCAGGTCACCATGAAAACCGCT | |
| | AAGGTGGCCGCCAGCGATTGGACCTTTCTGCACTGCCTGCCCAGAAAGCCCGAAG | |
| | AGGTGGACGACGAGGTCTTCTACTCTCCCAGAAGCCTGGTGTTTCCCGAAGCTGA | |
| | GAATAGGAAGTGGACAATTATGGCAGTGATGGTGTCCCTGCTGACTGATTATTCTC | |
| | CTCAACTGCAGAAACCTAAATTTTGATAAgaattc | |
| hOTC | ATGCTGTTCAACCTGCGAATCCTGCTGAACAACGCCGCTTTTCGGAACG | 13 |
| CO21 | GGCACAACTTTATGGTGAGGAACTTTCGCTGCGGACAGCCCCTCCAGAA | |
| (ORF) | TAAGGTCCAGCTGAAGGGCAGGGACCTGCTGACCCTGAAAAATTTCACA | |
| | GGGGAGGAAATCaaatacatgctgtggctgagcgccgatctgaagttca | |
| | gaatcaagcagaagggcgagtacCTGCCTCTGCTCCAGGGCAAAAGCCT | |
| | GGGGATGATCTTCGAAAAGCGCAGTACTCGGACCAGACTGTCAACCGAG | |
| | ACTggcttcgctctgctgggaggccacccttgcttcctgacaacccagG | |
| | ACATTCACCTGGGAGTGAACGAGTCCCTGACCGACACTGCTCGCGTCCT | |
| | GAGCTCTatggccgacgccgtgctggctcgggtgtacaaacagtccgac | |
| | ctggataccctggccaaggaagcttccATCCCCATCATCAACGGCCTGA | |
| | GCGACCTGTACCACCCCATCCAGATCCTGGCCGACTACCTGACCCTGCA | |
| | GGAGCACTACAGCAGCCTGAAGGGCCTGACCCTGAGCTGGATCGGCGAC | |
| | GGCAACAATATCCTGcactctattatgatgtctgccgccaagtttggaa | |
| | tgcacctgcaggctgctacccctaaaggctacgaacccgatgcctctgt | |
| | gacaaagctggctgaacagtacgccaaagagaacggcacaaagctgctg | |
| | ctgaccaacgaccctctggaggccgctcacggaggcaacGTGCTGATCA | |
| | CCGATACCTGGATTAGTATGGGACAGGAGgaagagaagaagaagcggct | |
| | gcaggccttccagggctaccaggtcaccatgaaaaccgctaaggtggcc | |
| | gccagcgatTGGACCTTTCTGCACTGCCTGCCCAGAAAGCCCGAAGAGG | |
| | TGGACGACGAGGTCTTCTACTCTCCCAGAAGCCTGGTGTTTCCCGAAGC | |
| | TGAGAATAGGAAGTGGACAATTATGGCAGTGatggtgtccctgctgact | |
| | gattattctcctcaactgcagaaacctaaattttga | |
| hOTC- | MLFNLRILLNNAAFRNGHNFMVRNFRCGQPLQNKVQLKGRDLLTLKNFT | 14 |
| 001_cds_(WT) | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVEYSPRSLVFPEAENRKWTIMAVMVSLLT | |
| | DYSPQLQKPKF* | |
| hOTC-CO1 | MLFNLRILLNNAAFRNGHNFMVRNFRCGQPLQNKVQLKGRDLLTLKNFT | 15 |
| | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVEYSPRSLVFPEAENRKWTIMAVMVSLLT | |
| | DYSPQLQKPKF* | |
| hOTC-CO2 | MLFNLRILLNNAAFRNGHNFMVRNFRCGQPLQNKVQLKGRDLLTLKNFT | 16 |
| | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVEYSPRSLVFPEAENRKWTIMAVMVSLLT | |
| | DYSPQLQKPKF* | |
| hOTC-CO3 | MLFNLRILLNNAAFRNGHNFMVRNFRCGQPLQNKVQLKGRDLLTLKNFT | 17 |
| | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVEYSPRSLVFPEAENRKWTIMAVMVSLLT | |
| | DYSPQLQKPKF* | |
| hOTC-006 | MLFNLRILLNNAAFRNGHNFMVRNFRCGQPLQNKVQLKGRDLLTLKNFT | 18 |
| | GEEIKYMLWLSADLKFRIKQKGEYLPLLQGKSLGMIFEKRSTRTRLSTE | |
| | TGFALLGGHPCFLTTQDIHLGVNESLTDTARVLSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVFYSPRSLVFPEAENRKWTIMAVMVSLLT | |
| | DYSPQLQKPKF* | |
| hOTC-CO6-1 | MLFNLRILLNNAAFRNGHNFMVRNFRCGQPLQNKVQLKGRDLLTLKNFT | 19 |
| | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVFYSPRSLVFPEAENRKWTIMAVMVSLLT | |
| | DYSPQLQKPKF* | |
| hOTC-CO7 | MLFNLRILLNNAAFRNGHNFMVRNFRCGQPLQNKVQLKGRDLLTLKNFT | 20 |
| | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVFYSPRSLVFPEAENRKWTIMAVMVSLLT | |
| | DYSPQLQKPKF* | |
| hOTC-CO9 | MLFNLRILLNNAAFRNGHNFMVRNFRCGQPLQNKVQLKGRDLLTLKNFT | 21 |
| | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVFYSPRSLVFPEAENRKWTIMAVMVSLLT | |
| | DYSPQLQKPKF* | |
| hOTC-CO9-1 | MLFNLRILLNNAAFRNGHNFMVRNFRCGQPLQNKVQLKGRDLLTLKNFT | 22 |
| | GEEKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVFYSPRSLVFPEAENRKWTIMAVMVSLLT | |
| | DYSPQLQKPKF* | |
| hOTC-CO9-2 | MLFNLRILLNNAAFRNGHNFMVRNFRCGQPLQNKVQLKGRDLLTLKNFT | 23 |
| | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVFYSPRSLVFPEAENRKWTIMAVMVSLLT | |
| | DYSPQLQKPKF* | |
| hOTC-CO18 | MLFNLRILLNNAAFRNGHNFMVRNFRCGQPLQNKVQLKGRDLLTLKNFT | 24 |
| | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVFYSPRSLVFPEAENRKWTIMAVMVSLLT | |
| | DYSPQLQKPKF* | |
| hOTC-CO21 | MLFNLRILLNNAAFRNGHNFMVRNFRCGQPLQNKVQLKGRDLLTLKNFT | 25 |
| | GEEIKYMLWLSADLKFRIKQKGEYLPLLQGKSLGMIFEKRSTRTRLSTE | |
| | TGFALLGGHPCFLTTQDIHLGVNESLTDTARVLSSMADAVLARVYKQSD | |
| | LDTLAKEASIPIINGLSDLYHPIQILADYLTLQEHYSSLKGLTLSWIGD | |
| | GNNILHSIMMSAAKFGMHLQAATPKGYEPDASVTKLAEQYAKENGTKLL | |
| | LTNDPLEAAHGGNVLITDTWISMGQEEEKKKRLQAFQGYQVTMKTAKVA | |
| | ASDWTFLHCLPRKPEEVDDEVFYSPRSLVFPEAENRKWTIMAVMVSLLT | |
| | DYSPQLQKPKF* | |

Bold caps indicate Start and Stop codon, small caps indicate the Kozak sequence.

### Example 3: In Vivo Liver-Targeting Studies

In vitro and in vivo studies were conducted in mice and non-human primates to screen several AAV capsid variants for the ability to target the liver with high efficiency. Additional preclinical studies were conducted to assess the safety and efficiency of the combination of AAV vectors and synthetic nanocarriers encapsulating rapamycin, demonstrating the feasibility of developing a therapeutic approach that allows for repeat dosing in diseases with early lethality.

### Example 4: In Vitro Testing of AAV8-hOTC-CO Constructs

The human hepatocellular carcinoma cell line HUH7 was used to evaluate the expression levels of AAV8-hOTC-CO constructs. The AAV8-hOTC-CO1 (C01), AAV8-hOTC-CO2 (CO2), AAV8-hOTC-CO3 (CO3), AAV8-hOTC-006 (CO6), AAV8-hOTC-CO7 (CO7), AAV8-hOTC-009 (CO9) construct plasmids, together with a pGFP plasmid as an internal control, were transiently co-transfected in HUH7 cells with Lipofectamine (Lipofectamine 2000, Thermo Fisher Scientific). Twenty-four hours post-transfection, total protein lysates were prepared and analyzed for OTC expression by Western blot analysis.

Results showed an overall increase of OTC protein expression for all the engineered sequences over the hOTC-wt (WT) construct (FIG. 14). CO6 was the most efficient construct, with an increase in expression efficiency that was about 5-fold higher than the WT construct, followed by CO3 and CO7, which had approximately 2.5-fold higher expression compared to the WT and CO1 constructs.

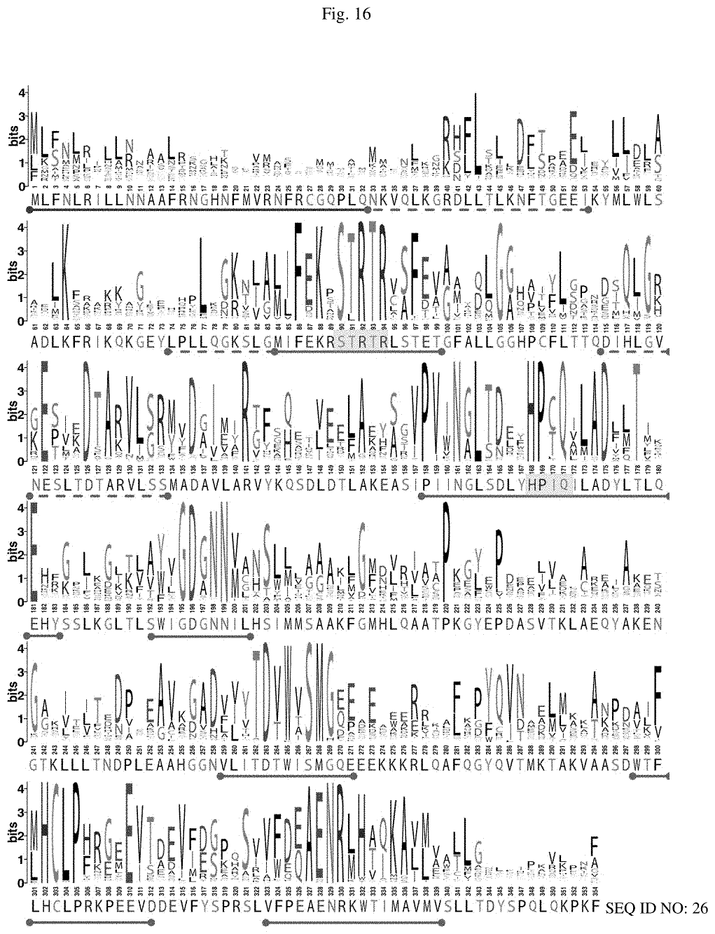

Two strategies were adopted to generate additional AAV8-hOTC-CO constructs with improved translatability: 1) the OTC CO6 and CO9 constructs were “re-optimized” by using bioinformatics algorithms (Table 2); and 2) based on the analysis of the most conserved regions of the OTC protein among species (FIG. 16), these regions were selected and shuffled among the most efficient AAV8-hOTC-CO constructs.

TABLE 2
hOTC-CO Sequence Descriptions

| Name | Description |
|---|---|
| hOTC-wt | WT human OTC coding sequence from gene bank |
| hOTC-CO1 | CO LW4 sequence (Wang, L, 2012, Molecular Genetics |
| | and Metabolism) |
| hOTC-CO2 | CO1 optimized by removing predicted cryptic splice sites |
| hOTC-CO3 | CO1 optimized by removing additional sites |
| hOTC-CO6 | Optimization made by JCat algorithm |
| hOTC-CO7 | Optimization made by GeneArt |
| hOTC-CO9 | Optimization made by DNA 2.0 |
| hOTC-CO6-1 | CO6, re-optimized by GeneArt |
| hOTC-CO9-1 | CO9, re-optimized by GeneArt |
| hOTC-CO9-2 | CO9, re-optimized by IDT |
| hOTC-CO18 | Chimeric sequence based on the shuffling the conserved |
| | regions between CO1, CO3, and CO6 |
| hOTC-CO21 | Chimeric sequence based on the shuffling the conserved |
| | regions between CO1, CO3, and CO6 |

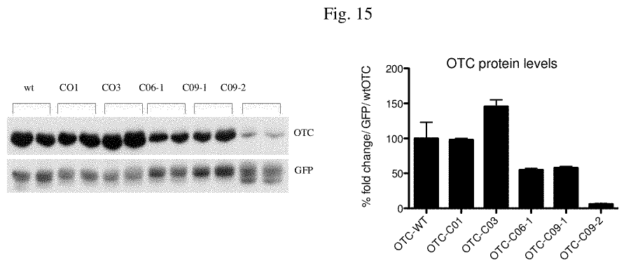

A group of 3 new codon-“re”-optimized constructs was tested (CO6-1, CO9-1, and CO9-2). HUH7 cells were transfected with the WT, CO1, CO3, CO6-1, CO9-1, CO9-2 constructs. The OTC protein expression levels of the CO6-1, CO9-1, and CO9-2 proteins were significantly reduced compared to WT, CO1, and other previously-tested constructs (FIG. 15).

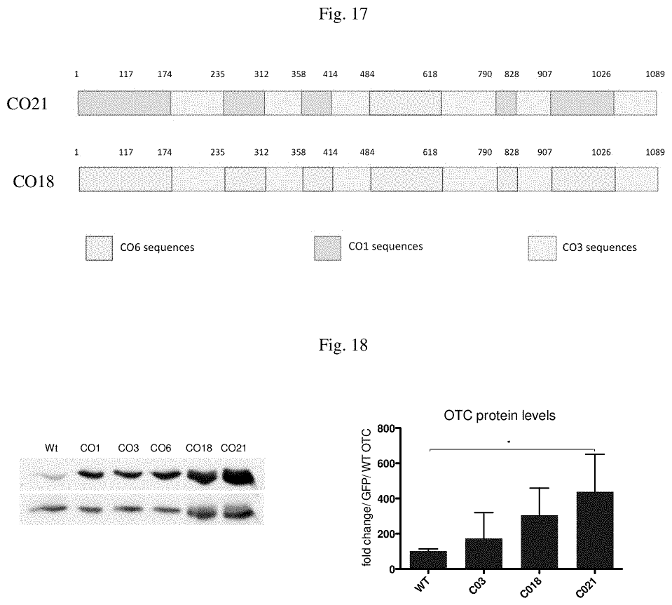

A third group of codon-optimized OTC sequences was generated in order to maintain a more efficient product. Functional analysis of the OTC ORF sequences were analyzed in order to identify the protein domains and conserved regions among species. These regions were shuffled among the CO1, CO3, and CO6 sequences to obtain the CO18 and CO21 sequences (FIG. 17). The CO18 and CO21 constructs were the most efficient in increasing OTC protein levels up to 5-6 fold higher than WT (FIG. 18). CO21 was selected as the candidate for OTC deficiency gene therapy.

The intracellular localization of the WT, CO1, and CO3 constructs to mitochondria was tested in HUH7 cells. HUH7 cells were transfected with the WT, CO1, and CO3 constructs and after 24 hours, the cells were stained with a mitochondrial marker (MitoTracker® Red CMXRos, Invitrogen) and anti-OTC antibody (Abcam ab203859). The resulting preparation was analyzed by confocal microscopy. The localization of all hOTC constructs was in mitochondria, as demonstrated by its strong co-localization with the mitochondrial marker (FIG. 9).

### Example 5: In Vivo Testing of AAV8-hOTC-CO Constructs

Two mouse animal models were used for the studies described herein: wild-type (WT) C57Bl/6 mice and OTC^{spf-ash} mice from Jackson Laboratory (B6EiC3Sn a/A-Otc^{spf-ash}, stock number 001811). After in vitro evaluation, the AAV8-hOTC-CO constructs were tested in vivo in WT C57Bl/6 mice, and the most efficient constructs were tested in the OTC^{spf-ash} mice. These experiments included comparison of the codon-optimized constructs with wild-type hOTC.

The AAV8-hOTC-CO constructs were tested in adult eight-week old male and female mice that were randomly assigned to treatment groups. Mice were treated with a single tail vein injection. Five different doses (5.0E12 vg/kg, 1.25E12 vg/kg, 1.0E12 vg/kg, 5.0E11 vg/kg, and 2.5E11 vg/kg) were tested to produce substantial OTC protein expression. In fact, the level of exogenous hOTC expression was tested to be high enough to limit the interference of endogenous OTC in analysis.

After treatment, mice were sacrificed at a specific time, and livers were collected and analyzed for OTC protein levels, OTC catalytic activity, and quantification of viral genomes per cell. Genome viral copies were determined by qPCR of genomic DNA extracted from liver powder using a commercial kit (Promega Wizard™ Genomic DNA Purification Kit). Measurement of viral genomes was repeated three times from the same DNA preparation and the average values are reported.

Ten milligrams of liver powder was lysed with 200 μl of mitochondria buffer (0.5% Triton, 10 mM HEPES, pH 7.4, 2 mM dithiotreitol) using an automatic homogenizer. Cellular debris was eliminated by centrifugation at maximum speed for 10 minutes and total protein concentration was determined by Bradford assay. Western blot analysis was performed by loading equal amounts of proteins (10 μg) in a 10% SDS-PAGE gel, which was then transferred onto nitrocellulose and incubated with the α-OTC antibody (Abcam ab203859, dilution: 1:3,000 in 5% milk-PBST). Anti-tubulin or GAPDH were used as loading controls.

OTC enzyme activity was measured three times. One microgram (1 μg) of total liver protein was incubated with 175 μl of freshly prepared Reaction Buffer (5 mM ornithine, 15 mM carbamyl phosphate, 270 mM triethanolamine, pH 7.7) for 30 minutes at 37° C. The reaction was stopped with 62.5 μL of 3:1 phosphoric acid:sulfuric acid solution. 12.5 μL of 3% 2,3-butanedione monoxime were then immediately added to the reaction, and the reactions were incubated at 95° C. for 15 minutes, protected from light. Samples were transferred to a 96-well plate and absorbance was measured at 490 nm. The reaction was performed in duplicate, and average values are reported. Protein levels and enzyme activity were normalized by the viral genome values.

Testing in WT C57Bl/6 Mice

The CO1, CO2, CO3, CO6, CO7, CO9, and WT constructs were tested in WT C57Bl/6 mice that were randomly assigned to treatment groups. Mice were treated with a single tail vein injection. The experimental groups and doses are as in Tables 13-15.

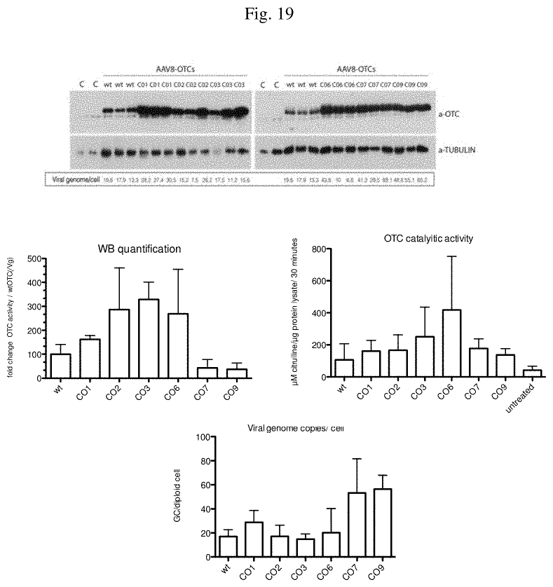

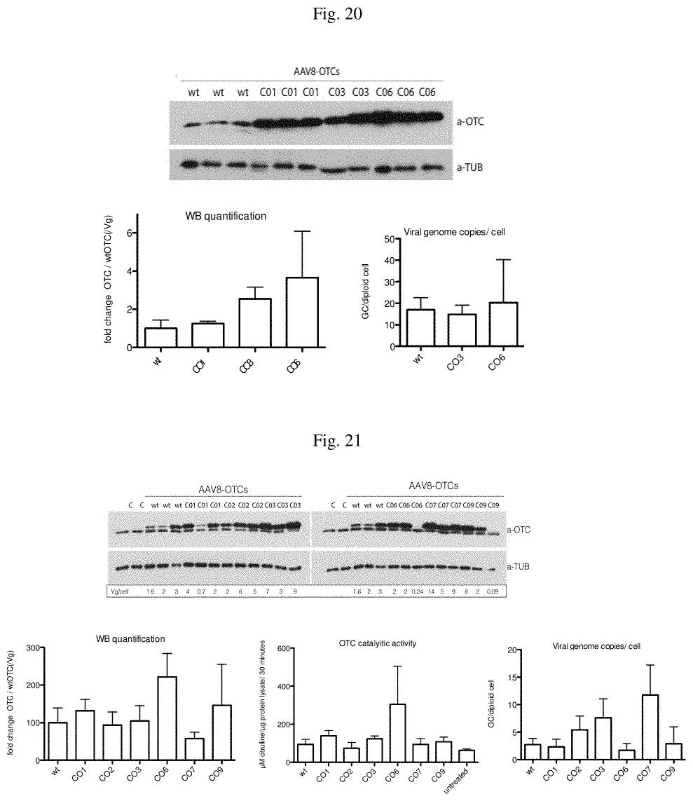

Transduction of male WT C57Bl/6 mice with a high dose (5.0E12 vg/kg) resulted in CO3 and CO6 being the most efficient constructs, both in terms of protein expression and activity compared to CO1 and WT constructs (FIGS. 19-20, Tables 3-6). In particular, mice injected with CO3 construct had 3-4 fold higher liver OTC levels and activity than mice injected with WT at equivalent viral genome copy concentrations (FIGS. 19-20, Tables 3-6). Furthermore, mice injected with CO6 had 4-6 fold higher liver OTC levels and activity than mice injected with WT (FIGS. 19-20). Viral genomes copies were consistent to protein levels and activity (FIG. 19).

Transduction of male mice with a lower dose (1.25E12 vg/kg) confirmed that the CO6 construct was the most efficient, showing 3-4 fold higher OTC activity than the WT version (FIG. 21, Tables 7-9). The CO3 construct resulted in 1.5-fold higher liver OTC activity than mice injected with WT at equivalent viral genome copies (FIG. 21, Tables 7-9).

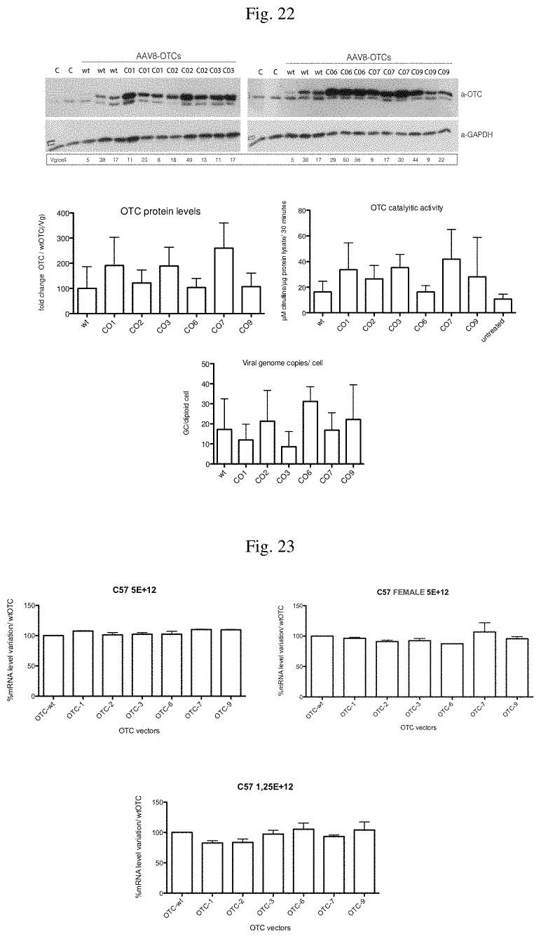

Conversely, transduction of female C57Bl/6 mice showed higher variability and a lower viral genome load in some animals in comparison to males (FIG. 22, Tables 10-12). Indeed, reduced AAV transduction in female mice has already been reported (Davidoff et al., 2003). Female C57Bl/6 mice injected with 5.0E12 vg/kg of CO3 had 3-4 fold higher OTC expression and activity than WT (FIG. 22, Tables 10-12). When measuring the hOTC mRNA levels in the liver of transduced mice, there were no statistical differences between different constructs, suggesting that codon-optimization did not affect gene transcription efficiency (FIG. 23).

Altogether, these data suggest that HUH7 transfection is a reliable test method to screen codon-optimized constructs, and that the codon-optimization strategy was effective at producing an engineered hOTC cassette that can be more efficient at transgene expression than wild-type transgenes in vivo. In particular, CO3 and CO6 were confirmed to be highly efficient in producing elevated levels of catalytically active OTC protein.

Testing in OTC^{spf-ash} Mice

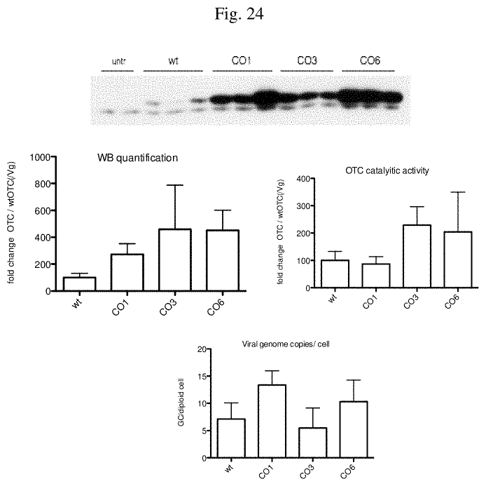

A second batch of AAV-hOTC-CO constructs were prepared in order to perform experiments in OTC^{spf-ash} mice. This second batch was first tested in male WT C57BL/6 mice in order to compare the transduction efficiency with that of the first batch (FIG. 24, Tables 16-19). Similar results were obtained for protein expression, OTC catalytic activity, and viral genome copies per cell as in the previous experiments.

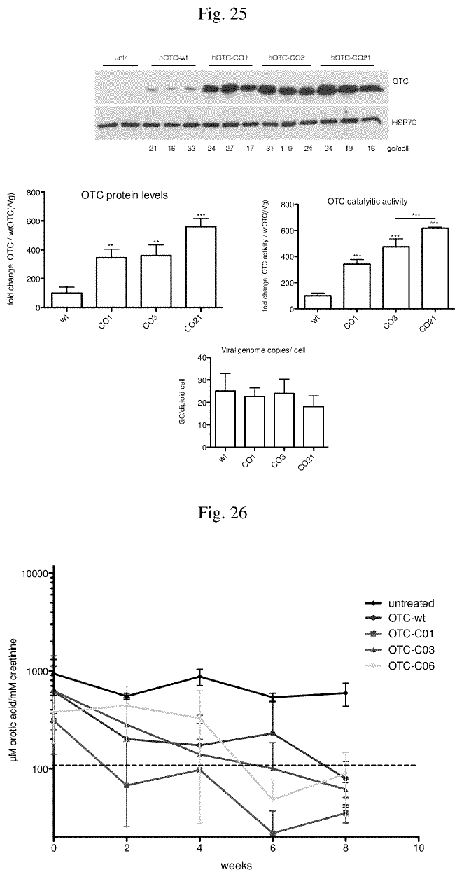

Based on in vitro results, the CO21, CO1, and CO3 constructs were tested in WT C57BL/6 mice. AAV8-OTC-CO21 was the most efficient at increasing protein expression, with 6-8 fold higher OTC expression and catalytic activity compared to the WT construct, and 2-fold higher catalytic activity than the CO3 construct (FIG. 25, Tables 20-23).

The WT, CO1, CO3, and CO6 constructs were tested in adult eight week-old OTC^{spf-ash} mice (Table 24). The OTC^{spf-ash} mice are an established model of OTCd and are widely used in clinical studies (Moscioni, et al., 2006; Cunningham, et al., 2011; Wang, et al., 2012). The OTC^{spf-ash} mice carry a hypomorphic guanine to adenosine mutation in the last nucleotide of exon 4 of the OTC gene, located on the X-chromosome. This leads to aberrant silencing and production of only 5% of correctly spliced mRNA and 5-10% residual OTC enzymatic activity. Hemizygous OTC^{spf-ash} male mice are viable, but show reduced lifespan when maintained on normal diet. Clinically, OTC^{spf-ash} mice present growth retardation, sparse fur, hyperammonemia, and increased urinary orotic acid. Upon nitrogen-load growth challenge, these mice develop ammonia-induced encephalopathy. The absence of severe neurological damage in mice on normal diet indicates that these mice can be used as a model for delayed onset OTC deficiency, a milder form of the disease.

In OTC^{spf-ash} mice, the minimum group size necessary to reach statistical significant of the experiments was calculated using the G*Power software (Version 3.1.9.3), considering α=0.05, 1−β=0.8, two tails, similar group size, effect size high, considering a value of urinary orotic acid in untreated OTC^{spf-ash} mice of 600 μmol/mmol creatinine, and in treated mice of 200 μmol/mmol creatinine±SD of 100 μmol orotic acid/mmol creatinine, which corresponds to a mild effect in correction of the defect (levels of wild-type mice are about 60-100 μmol orotic acid/mmol creatinine). The minimum size group calculated was 3 mice, and 4 were used for experiments described herein.

Urinary orotic acid was used to evaluate phenotype correction after AAV8-hOTC construct transduction. Urinary orotic acid was quantified by stable-isotope-dilution liquid chromatography-mass spectrometry as described herein. Urine was collected in 1.5 mL tubes and centrifuged for 1 minute at the maximum speed for clarification. 10 μL of urine was diluted in 90 μL of stable isotope buffer (0.2 mM 1,3-(¹⁵N₂) orotic acid in 1.25 mM NH4OAc). Samples were analyzed for orotic acid using liquid chromatography/tandem mass spectrometry using the transition 111.1>155.1 and 157>113 for native and stable isotope orotic acid, respectively. The mobile phase consisted of acetonitrile-0.1% Formic acid. Results were standardized against creatinine levels, measured using enzymatic commercial kit (Mouse Creatinine Assay kit, 80350, Crystal Chem). Adapted from Cunningham, et al., 2009.

Urinary orotic acid in urine was measured 1 day before injection and every 2 weeks after injection. All rAAV8-hOTC constructs injected into OTC^{spf-ash} mice were able to reduce orotic acid levels, restoring physiological levels at 8 weeks post-injection. All rAAV vectors resulted in the normalization of urinary orotic acid 8 weeks after vector delivery, with CO1 and CO3 having higher kinetics of returning orotic acid at 2 weeks post-treatment (FIG. 26, Table 25).

Plasma ammonia level was also measured; however, due to its fluctuations in the OTC^{spf-ash} mouse blood, it cannot be considered by itself to be a highly reliable test parameter. 50 μL of blood was collected by submandibular puncture in EDTA-containing tubes and immediately placed on ice. Plasma was extracted by centrifugation at 3,000 rcf for 15 minutes and ammonia was immediately measured using a commercial kit (Ammonia assay kit, MAK310, Sigma).

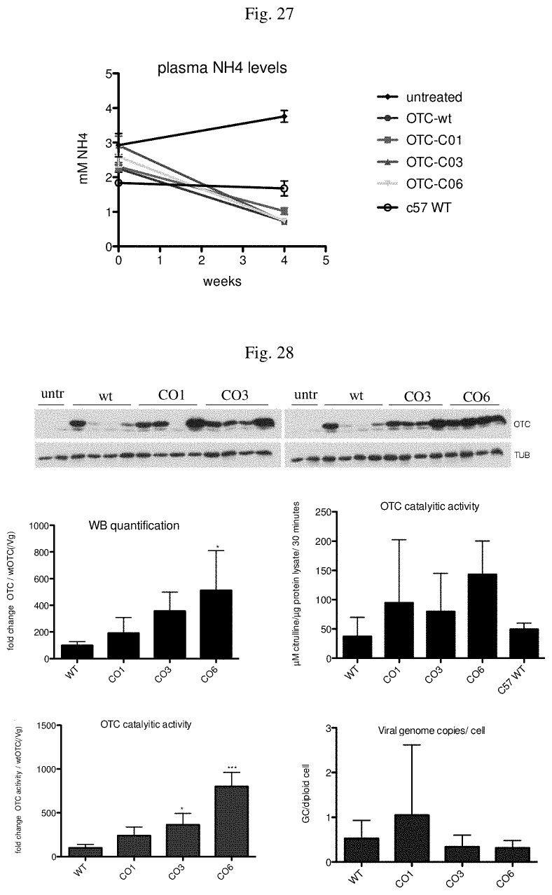

Four weeks after injections with WT, CO1, CO3, and CO6, ammonia levels were significantly reduced below the physiological level (FIG. 27, Table 26). Livers of male OTC_{spf-ash} mice were analyzed for protein expression, OTC catalytic activity, and viral genome copy number. In line with WT C57Bl/6 mice, CO3 and CO6 significantly improved transduction efficiency, mediating a 4-fold and 6-fold increase in transgene expression, respectively, when normalized to viral genome copies (FIG. 28, Tables 27-29). OTC catalytic activity showed a strong correlation with protein levels (FIG. 28).

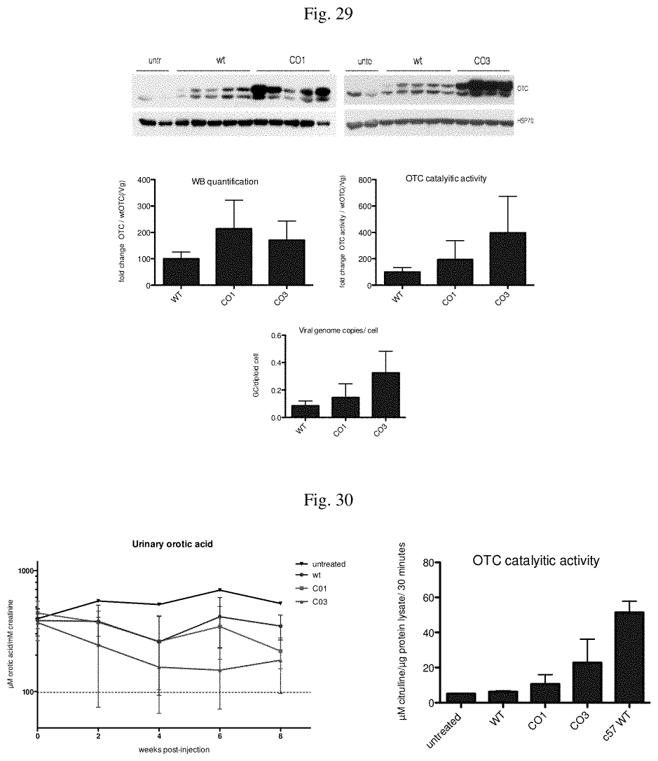

Hemizygous OTC^{spf-ash} male mice injected with 5.0E11 vg/kg dose were analyzed following the same experiment rationale as the above-described experiment. Orotic acid was measured 1 day before injection and periodically every 2 weeks after viral administration. Mice were sacrificed 8 weeks after viral administration and livers were collected to evaluate OTC protein expression, catalytic activity and viral genome copy number. Although mice injected with CO3 had a significant 3-4 folds increase in liver OTC expression and catalytic activity compared to WT treated animals, the overall viral genome copies and, consequently, hOTC expression and activity were importantly reduced compared to previously-described experiment (FIGS. 29-30, Tables 34-36). Orotic acid levels were reduced but did not reach physiological normal values (FIG. 30, Tables 37-38).

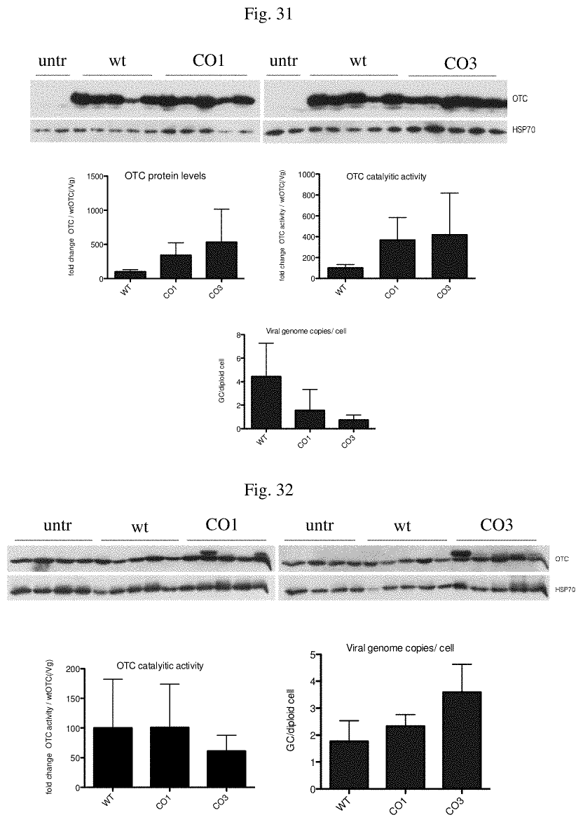

Hemizygous OTC^{spf-ash} male mice injected with an AAV8 dose of 1.0E12 vg/kg were sacrificed after 8 weeks and the livers were collected to evaluate OTC protein expression, OTC catalytic activity and viral genome copy number. This experiment confirmed the improved efficacy of CO3 in terms of protein expression and catalytic activity, when compared to WT and CO1 constructs (FIG. 31, Tables 39-41).

Heterozygous female OTC^{spf-ash} mice injected with WT, CO1, and CO3 constructs in two doses (5.0E11 vg/kg and 1.0E12 vg/kg) had a reduced efficiency and an increased variability, as already observed in WT C57Bl/6 mouse experiments (FIG. 32, Tables 42-43). However, hOTC protein quantification and activity analysis in the liver of 1.0E12 vg/kg treated mice confirmed the CO3 construct as the most efficient construct, having up to 4-5 fold increased efficiency compared to WT, and 1.5-2-fold increase with respect to CO1 (FIG. 33, Tables 44-46).

The CO21 construct was then evaluated in OTC^{spf-ash} mice, in a side-by-side comparison experiment, together with WT and CO3 constructs, at an initial dose of 1.0E12 vg/kg. Moreover, to further characterize the CO21 construct as a potential clinical candidate, a dose finding study for CO21 was performed, using three different doses: 1.0E12 vg/kg, 5.0E11 vg/kg, and 2.5E11 vg/kg (Tables 47-49). The dose finding experiment was conducted in a side-by-side comparison with the WT construct.

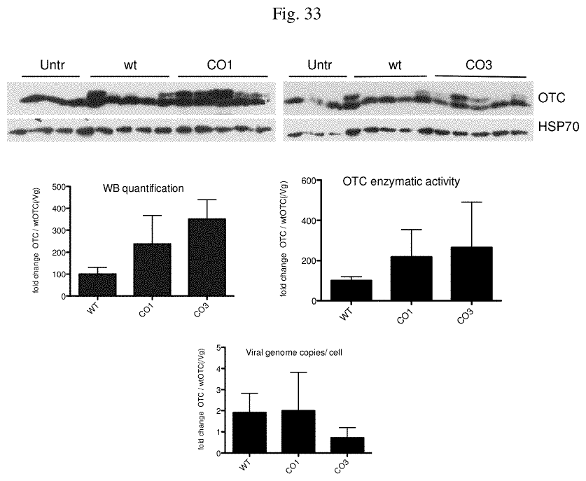

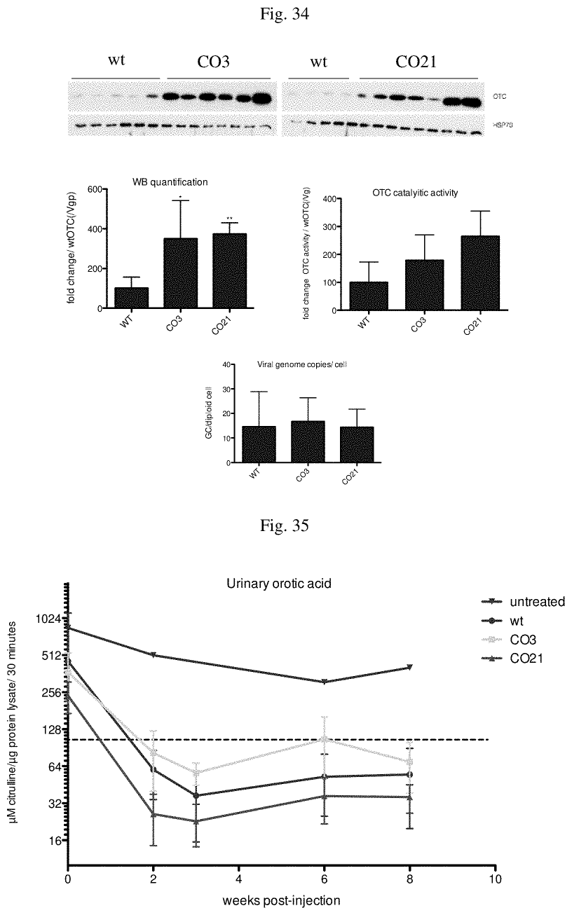

The analysis of OTC^{spf-ash} mice injected with 1.0E12 vg/kg dose showed that all three constructs were able to correct the OTC phenotype, reducing urinary orotic acid concentration to physiological levels 2 weeks after injection (FIG. 35, Table 53). However, CO21 demonstrated the best kinetic and efficiency, showing a more stable reduction over time (FIG. 35). OTC protein levels and catalytic activity analysis in the liver of mice injected with CO3 or CO21 showed a comparable improvement in comparison to WT (FIG. 34, Tables 50-52).

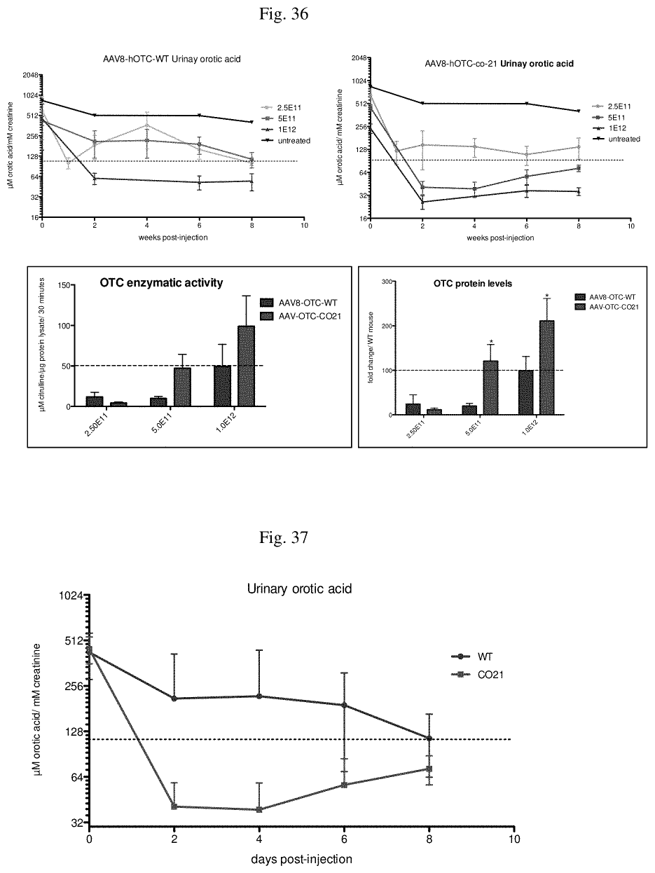

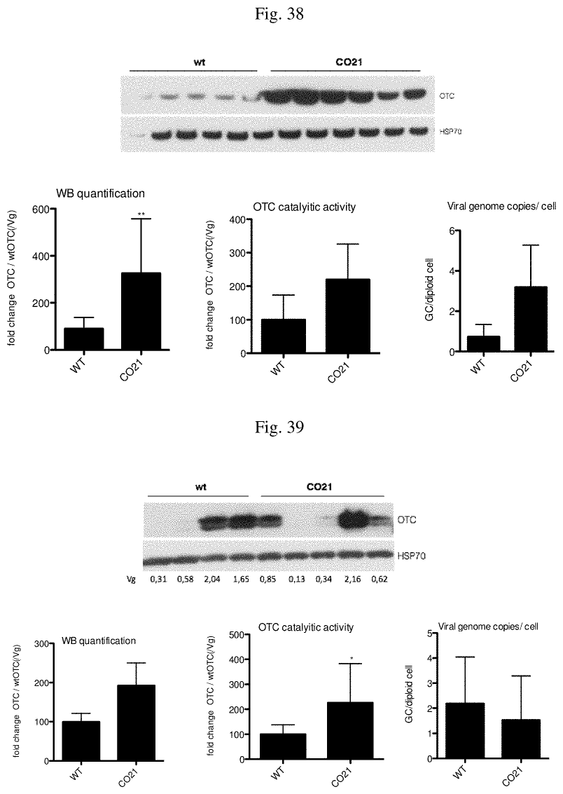

The analysis of urinary orotic acid from OTC^{spf-ash} mice injected with the intermediate dose (5.0E11 vg/kg) demonstrated the stronger efficacy of CO21 in correcting the phenotype by reducing the orotic acid levels to the physiological range. Conversely, OTC^{spf-ash} mice injected with WT at the same dose produce sub-therapeutic effects, with orotic acid above physiological levels (FIG. 36, 37, Tables 54-57). OTC protein levels and catalytic activity analysis confirmed the orotic acid measurement. In fact, mice treated with CO21 had 4-fold higher liver hOTC expression that appeared to correct the OTC phenotype (FIGS. 36, 38, Tables 55-57).

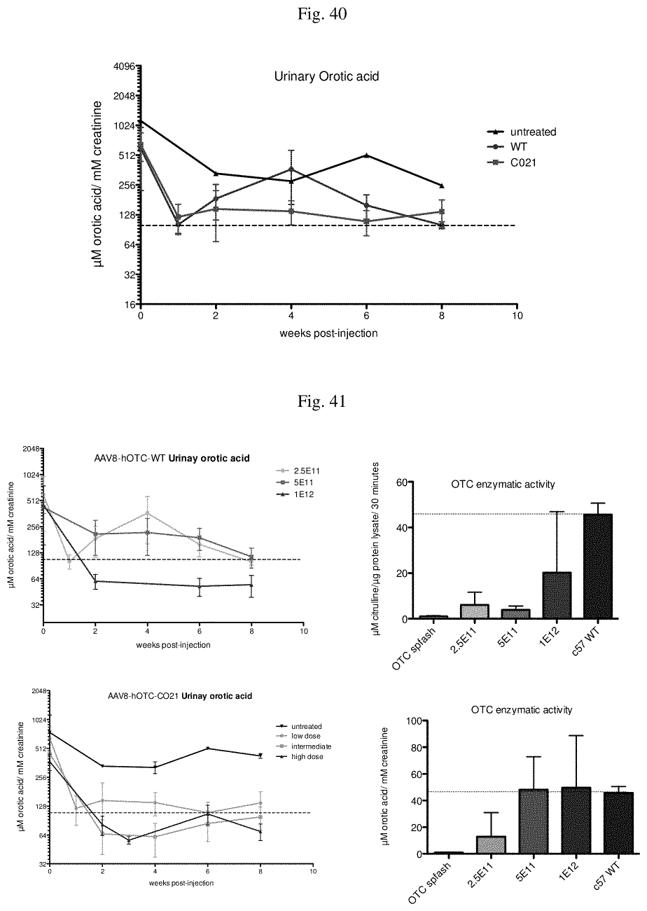

Finally, OTC^{spf-ash} mice injected with the lower dose (2.5E11 vg/kg) of WT and CO21 (2.5E11) were found to be partially corrected. As shown in the urinary orotic acid graph, a significant reduction of orotic acid was observed at 2 weeks after injection, even though levels were fluctuating around the pathological threshold and unstable (FIG. 40, Table 61). CO21 maintained an higher hOTC expression efficiency, around 3-fold, than WT (FIG. 39, Tables 58-60).

Altogether, these data suggest that the CO21 is about 5-fold more efficient than WT in expressing a catalytically active hOTC in liver. Due to the increased expression efficiency, CO21 provides a therapeutic effect at the dose of 5.0E11 vg/kg, providing enough protein to correct the OTC^{spf-ash} phenotype (FIG. 41, Table 62). 5.0E11 vg/kg is a sufficient dose to restore physiological levels of OTC protein and reduce urinary orotic acid to normal values in OTC^{spf-ash} mice. Thus, the AAV8-hOTC-CO21 construct mediates an efficient and safe correction of OTC deficiency in OTC^{spf-ash} mice.

### Example 6: In Vivo Ammonia Challenge

OTC^{spf-ash} mice have increased blood ammonia levels compared to wild-type mice. The efficiency of ammonia (NH₄) clearance from the blood was examined in OTC^{spf-ash} mice in an ammonia challenge experiment. OTC^{spf-ash} mice were injected with a single dose of 5.0E11 vg/kg of AAV8-hOTC-WT (WT) or AAV8-hOTC-CO21 (CO21) (Table 63). 4 and 8 weeks post-injection, the mice were subjected to an ammonia challenge experiment in which 7.5 mmol/kg of a 0.64M NH4C1 solution is injected intraperitoneally. B6EiC3Sn-WT (WT-CH3) mice were used as a control.

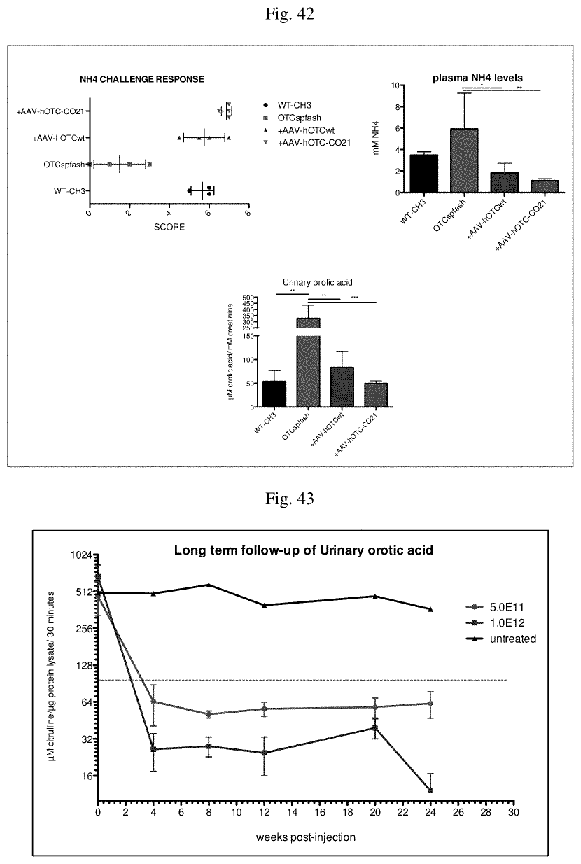

20 minutes after the NH₄Cl injection, mice were subjected to behavioral tests to assess ammonia (NH₄) crisis. A behavioral score was assigned to each mouse according to the scheme in Table 64 (FIGS. 42, 44). Ataxia was measured by subjecting the animals to the blind tunnel test. Mouse paws were dipped in non-toxic paint (one color for fore paws and a second color for hind paws), and the mouse was placed at one end of a blind tunnel (10 cm wide×50 cm long×10 cm high). The bottom of the tunnel was lined with white paper to analyze the gait. Response to sound was determined by placing the mouse 1.5 meters from a 100 db bell and observing the behavior after ringing the bell 3 times for 5 seconds each.

After the behavioral tests, 50 μl of blood was collected from the mice and ammonia was measured using a commercial kit (Ammonia assay kit, MAK310, Sigma). Urinary orotic acid was also measured.

TABLE 64
Behavioral Scoring Scale.

| Category | Description of behavior | Score |
|---|---|---|
| Intensity of | No seizure | 2 |
| Seizure | Myoclonus: spontaneous jerking | 1 |
| | Tonic-clonic seizures: rigid extensions of limbs | 0 |
| Ataxia | Normal gait | 2 |
| | Abnormal gait | 1 |
| | Unable to support itself: lying on a side | 0 |
| Response to | Normal, no response to sound | 3 |
| sound | Twitching of extremities | 2 |
| | Jumping | 1 |
| | Moribund or lying on a side | 0 |

The ammonia challenge experiment at 4 weeks post-injection demonstrated that CO21 is highly efficient in protecting OTC^{spf-ash} mice from ammonia challenge, as shown by the behavioral test score and by the NH4 levels measurement that were comparable to wt animals (FIGS. 42, 44, Tables 65-69). Moreover, the correction was maintained (stable) until 8 weeks from injection, when CO21 treated mice were still protected from NH4 and comparable to WT animals (FIGS. 42, 44). The WT construct was less efficient than CO21 in ammonia clearance, in particular during the second ammonia challenge experiment (FIG. 44), where the total behavioral score assigned was slightly above the scored assigned to WT animals. All the measured data are consistent with molecular analysis of OTC protein expression and activity (FIGS. 42-44).

### Example 7: Deletion of Enhancer Sequences Improves AAV8-hOTC-CO21 Safety In Vivo

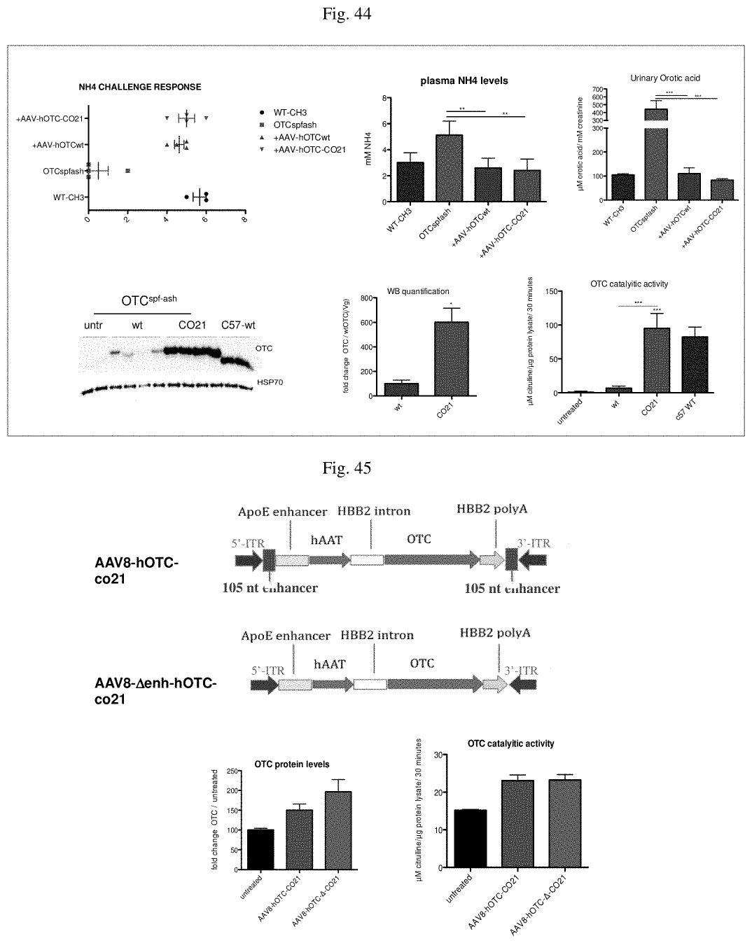

The AAV8-hOTC-CO21 construct contains 105 nucleotide (nt) enhancer sequences adjacent to the 5′ and 3′ inverted terminal repeats (ITRs). The enhancer sequences were deleted (AAV8-hOTC-Δ-CO21, also referred to as AAV8-hOTC-Δenh-CO21) to increase the safety of the AAV8-hOTC-CO21 construct in vivo. Human hepatocytes were transduced with AAV8-hOTC-CO21 or AAV8-hOTC-Δ-CO21. AAV8-hOTC-Δ-CO21 showed increased protein levels and similar catalytic activity levels compared to AAV8-hOTC-CO21 (FIG. 45).

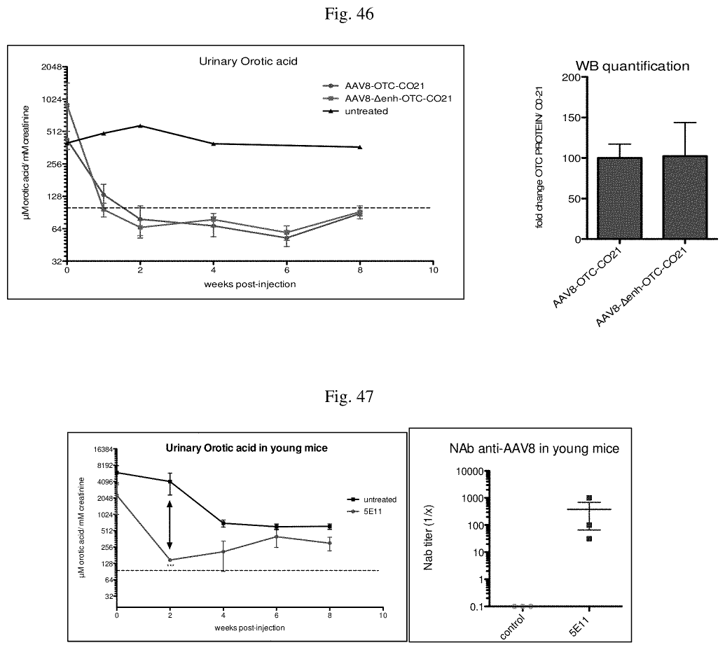

OTC^{spf-ash} mice were injected with either AAV8-hOTC-CO21 or AAV8-hOTC-Δ-CO21. The AAV8-hOTC-Δ-CO21 construct reduced urinary orotic acid and produced protein levels that were similar to the AAV8-hOTC-CO21 construct (FIG. 46).

### Example 8: Reducing Immunogenicity in Mice

Pediatric patients present three main challenges to gene therapy: vector loss over time as the patient grows, administration of AAVs causes production of neutralizing antibodies that limit the possibility to re-treat patients, and cellular immune responses to AAV leading to liver inflammation and loss of transgene expression.

AAV8 constructs encoding transgenes (e.g., luciferase, alpha-acid glucosidase, Factor IX coagulation factor) were injected into WT C57BL/6 mice or non-human primates (Macaca fasicularis) in the presence of synthetic nanoparticles to examine the generation of antibodies against the AAV8-transgene proteins.

For non-human primate experiments, three male cynomolgus monkeys (Macaca fasicularis) were selected based on their lack of neutralizing AAV8 antibodies. At day 0, animals were randomized to the treatment groups and received intravenous infusion (30 ml/h of SVP[Rapa] (3 mg/kg of rapamycin, n=2 SVP[Rapa]#1 and SVP[Rapa]#2 or SVP[empty] (n=1) followed immediately by intravenous infusion of an AAV8-alpha-acid glucosidase (AAV8-Gaa) vector (2.0E12 vg/kg). One month later, each animal received a second infusion of SVP[Rapa] (3 mg/kg of rapamycin, n=2 SVP[Rapa]#1 and SVP[Rapa]#2 or SVP[empty] (n=1), followed by the infusion of AAV8-human Factor IX coagulation factor vector (AAV8-hFI.X) (2.0E12 vg/kg).

In mouse and non-human primate experiments, peripheral blood was collected and sera were isolated or immediately transferred to tubes containing citrates or EDTA to isolate plasma, at baseline and indicated time points. Spleen and inguinal lymph nodes were collected at necroscopy in fresh RPMI medium and diverse organs were collected and stored at −80° for further analysis.

Synthetic nanoparticles (SVPs) composed of the polymers polylactic acid (PLA) and polylactic acid-polyethylene glycol (PLA-PEG) were synthesized using the oil-in-water single emulsion evaporation method as in Kishimoto, et al., 2016, Nat. Nanotechnology and Maldonado, et al., 2015, PNAS. Briefly, rapamycin, PLA, and PLA-PEG block copolymer were dissolved in dichloromethane solution to form the oil-phase. The oil-phase was added to an aqueous solution of polyvinylalcohol in phosphate buffer followed by sonication. The emulsion thus formed was added to a beaker containing phosphate buffer solution and stirred at room temperature for 2 hours to allow the dicholormethane to evaporate. The resulting nanoparticles containing rapamycin were washed twice by centrifugation at 76,6000×g+4° C. and the pellet was resuspended in phosphate buffer solution. The bare nanoparticles without rapamycin were prepared in identical conditions without rapamycin.

Antibody measurement assays were performed using ELISA and in vitro neutralization assays as in Meliani, et al., 2018, Nature Communications, Mingozzi, et al., 2013, Sci. Transl. Med and Meliani, et al., 2017, Blood Adv. Briefly, for the ELISA assay, Nunc™ MaxiSorp™ plates (Thermo Fisher Scientific) were coated with AAV particles (2.0E12 particles/mL) and with serial dilution of purified immunoglobulin (IgG1, IgG2a, IgG2b, and IgG3 for murine samples; IgG and IgM for non-human primate samples) to generate a standard curve. After overnight incubation at 4° C., the plates were blocked with PBS-0.05% Tween-20 containing 2% bovine serum albumin (BSA) and appropriately diluted samples were plated in duplicate. Samples were incubated at room temperature for 3 hours. Plates were then washed, and a secondary antibody conjugated to HRP was added to the wells and incubated for 1 hour at 37° C. Plates were then washed and the presence of bound antibodies were detected using SIGMAFAST™ OPD substrate measuring absorbance at 492 nm.

Plasma levels of the human F.IX transgene were measured as in the ELISA assay described herein. The detection of hFI.X antigen levels in mouse plasma was performed using monoclonal antibodies against hF.IX (GAFIX-AP, Affinity Biologicals). In non-human primate samples, anti-hFIX antibody (MA1-43012, Thermo Fisher Scientified) and anti-hFiIX-HRP antibody (CL20040APHP, Tebu-bio) were used for coating and detection, respectively.

Selected serum samples were also analyzed for anti-AAV neutralizing antibody titer using an in vitro cell-based test as in Meliani, et al., 2015, Hum. Gene. Ther. Methods. Briefly, serial dilutions of heat-inactivated samples were mixed with a vector expressing luciferase and incubated for 1 hour. After incubation, samples were added to cells and residual luciferase expression was measured after 24 hours. The neutralizing titer was determined as the highest sample dilution at which at least 50% inhibition of luciferase expression was measured compared to a non-inhibition control. In this assay, a neutralizing antibody (Nab) titer of 1:10 represents the titer of a sample in which after a 10-fold dilution, a residual luciferase signal lower lower than 50% of the non-inhibition control is observed.

P30 juvenile OTC^{spf-ash} mice were injected with 5.0E11 vg/kg AAV8-hOTC-CO21 (CO21) to assess immunogenicity. Urinary orotic acid and neutralizing antibodies (NAb) were measured (FIG. 47). The urinary orotic acid concentration in mice injected with AAV8-hOTC-CO21 decreased to 2 weeks after injection, but subsequently increased. Neutralizing antibodies were also generated in OTC^{spf-ash} juvenile mice injected with AAV8-hOTC-CO21. The results presented herein demonstrate that vector loss occurs over time and that administration of AAVs causes production of neutralizing antibodies. This potentially limits the possibility to re-treat patients, and leads to cellular immune responses to AAV leading to liver inflammation and loss of transgene expression.

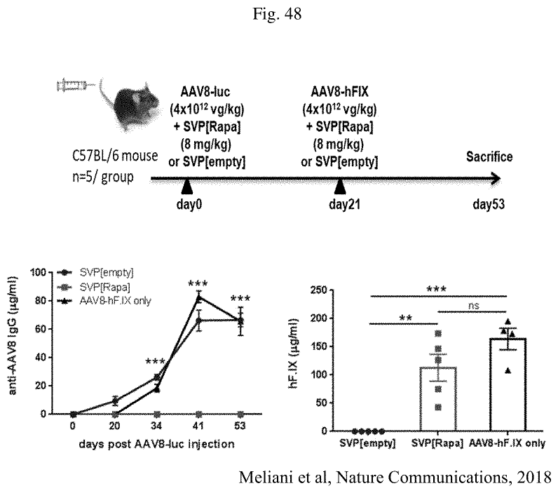

AAV8 constructs were packaged in synthetic viral particles (SVPs) containing the immunosuppressant rapamycin to examine the ability of the rapamycin (rapa) to suppress immunogenicity in vivo. C57BL/6 mice were injected with 4.0E12 vg/kg AAV8-luciferase and SVP[rapa] (8 mg/kg) or SVP[empty]. 21 days later, the mice were injected with 4.0E12 vg/kg AAV8-hFIX and SVP[rapa] (8 mg/kg) or SVP[empty]. The levels of anti-AAV8 IgG and hFIX were measured in the mice (FIG. 48). Administration of SVP[rapa] decreased anti-AAV8 IgG levels compared to mice administered SVP[empty] or AAV8-hFIX only. The levels of hFIX in mice administered SVP[rapa] were similar to mice administered AAV8-hFIX only, and significantly increased relative to mice administered SVP[empty] (FIG. 48).

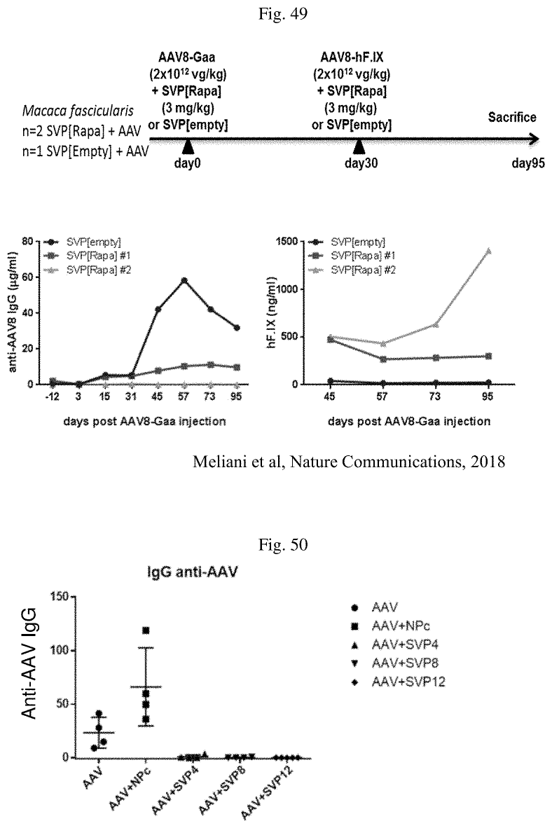

The immunogenicity of AAV8 constructs packaged in SVP[rapa] or SVP[empty] was further examined in non-human primates (Macaca fasicularis). The non-human primates were injected with either 2.0E12 vg/kg AAV8-Gaa and 3 mg/kg SVP[rapa] or SVP[empty]. 30 days later, the non-human primates were injected with 2.0E12 vg/kg AAV8-hFIX and 3 mg/kg SVP[rapa] or SVP[empty]. The levels of anti-AAV8 IgG and hFIX were measured in the non-human primates (FIG. 49). Administration of SVP[rapa] decreased anti-AAV8 IgG levels compared to non-human primates administered SVP[empty]. The levels of hFIX in non-human primates administered SVP[rapa] were increased relative to non-human primates administered SVP[empty]. The results presented herein sugges that concomitant administration of AAV vectors and and synthetic nanocarriers can increase transgene expression and decrease immune responses to the AAV vector.

### Example 9. SVP-Rapamycin Inhibits Anti-AAV8 IgG Response Against AAV8-OTC CO21 in OTC^{spf-ash} Mice

The effects of different doses of synthetic nanocarriers coupled to rapamycin (SVP-rapamycin) and the AAV8-OTC CO21 construct on AAV8 IgG response in OTC^{spf-ash} mice were examined. The OTC^{spf-ash} mice were dosed as follows: (1) AAV8-OTC CO21 alone (“AAV”), (2) AAV8-OTC CO21+empty nanoparticle control (“AAV+NPc”), (3) AAV8-OTC CO21+4 mg/kg SVP-Rapamycin (“AAV+SVP4”), (4) AAV8-OTC CO21+8 mg/kg SVP-Rapamycin (“AAV+SVP8”), or (5) AAV8-OTC CO21+12 mg/kg SVP-Rapamycin (“AAV+SVP12”) on day 0. Anti-AAV8 IgG antibody response was assessed at 2 weeks after dosing, and the results are shown in FIG. 50. As shown in the Figure, administration of the AAV9-OTC CO21 vector and synthetic nanocarriers comprising rapamycin inhibited the anti-AAV8 IgG response regardless of the dose of synthetic nanocarriers comprising rapamycin administered.

### Example 10

Presented in this Example are Tables 3-69 described in Examples 1-9.

TABLE 3
Western Blot Quantification of FIG. 19.

| | | | Band intensity | | | |
|---|---|---|---|---|---|---|
| | | | OTC/Tubulin | | | |
| Group | Virus name | Mouse | (normalized on | Fold change | | |
| (male) | (code) | code | Vg) | OTC-wt | Mean | Standard deviation |
| 1 | OTC-wt | A2901 | 0.03 | 0.74 | 100 | 41 |
| | (1668) | A2902 | 0.03 | 0.79 | | |
| | | A2903 | 0.06 | 1.47 | | |
| 2 | OT-C01 | A2904 | 0.07 | 1.79 | 162 | 15 |
| | (1669) | A2905 | 0.06 | 1.60 | | |
| | | A2906 | 0.06 | 1.48 | | |
| 3 | OTC-C02 | A2907 | 0.07 | 1.81 | 286 | 174 |
| | (1692) | A2908 | 0.19 | 4.87 | | |
| | | A2909 | 0.07 | 1.91 | | |
| 4 | OTC-C03 | A2910 | 0.10 | 2.62 | 329 | 73 |
| | (1678) | A2911 | 0.15 | 4.06 | | |
| | | A2912 | 0.12 | 3.18 | | |
| 1 | OTC-wt | A2901 | 0.05 | 0.67 | 100 | 38 |
| | (1668) | A2902 | 0.06 | 0.91 | | |
| | | A2903 | 0.10 | 1.41 | | |
| 5 | OTC-C06 | A2913 | 0.04 | 0.61 | 269 | 186 |
| | (1679) | A2914 | 0.22 | 3.27 | | |
| | | A2915 | 0.29 | 4.19 | | |
| 6 | OTC-C07 | A2916 | 0.06 | 0.83 | 43 | 35 |
| | (1680) | A2917 | 0.01 | 0.16 | | |
| | | A2918 | 0.02 | 0.29 | | |
| 7 | OTC-C09 | A2919 | 0.04 | 0.61 | 36 | 27 |
| | (1681) | A2920 | 0.01 | 0.08 | | |
| | | A2921 | 0.03 | 0.41 | | |

TABLE 4
OTC Catalytic Activity Quantification of FIG. 19.

| | | | OTC activity (μmol | | | | |
|---|---|---|---|---|---|---|---|
| | Virus | | citrulline/μg lysate/30 | | | | |
| Group | name | Mouse | minutes) | | | Individual | Group Mean ± |
| (male) | (code) | code | Exp 1 | Exp 2 | Exp 3 | Mean ± SD | SD |
| 1 | OTC-wt | A2901 | 45.36 | 112.6 | 88.6 | 82.2 ± 34.1 | 105.7 ± 81.1 |
| | (1668) | A2902 | 29.32 | 69.0 | 18.5 | 38.9 ± 26.6 | |
| | | A2903 | 35.14 | 274.6 | 278.2 | 196.0 ± 139.3 | |
| 2 | OT-C01 | A2904 | 77.44 | 116.9 | 218.2 | 137.5 ± 72.6 | 160.8 ± 25.8 |
| | (1669) | A2905 | 135.76 | 139.1 | 290.7 | 188.5 ± 88.5 | |
| | | A2906 | 119.72 | 135.4 | 214.0 | 156.4 ± 50.5 | |
| 3 | OTC- | A2907 | 59.94 | 154.6 | 211.6 | 142.0 ± 76.6 | 165.5 ± 36.0 |
| | C02 | A2908 | 70.14 | 197.1 | 353.7 | 207.0 ± 142.0 | |
| | (1692) | A2909 | 81.8 | 117.4 | 243.4 | 147.5 ± 84.9 | |
| 4 | OTC- | A2910 | 74.52 | 161.9 | 110.8 | 115.7 ± 43.9 | 250.5 ± 119.2 |
| | C03 | A2911 | 77.44 | 267.0 | 537.1 | 293.8 ± 231.0 | |
| | (1678) | A2912 | 164.92 | 306.1 | 554.9 | 342.0 ± 197.4 | |
| 5 | OTC- | A2913 | 182.42 | 161.2 | 311.7 | 218.4 ± 81.5 | 417.1 ± 194.9 |
| | C06 | A2914 | 115.34 | 386.1 | 773.5 | 425.0 ± 330.8 | |
| | (1679) | A2915 | 138.68 | 610.0 | 1075.3 | 608.0 ± 468.3 | |
| 6 | OTC- | A2916 | 204.3 | 156.0 | 235.2 | 198.5 ± 39.9 | 176.7 ± 49.9 |
| | C07 | A2917 | 147.42 | 200.2 | 288.5 | 212.0 ± 71.3 | |
| | (1680) | A2918 | 135.76 | 99.4 | 124.0 | 119.7 ± 18.6 | |
| 7 | OTC- | A2919 | 112.42 | 138.1 | 208.3 | 152.9 ± 49.6 | 136.4 ± 19.9 |
| | C09 | A2920 | 145.96 | 94.7 | 185.5 | 142.1 ± 45.5 | |
| | (1681) | A2921 | 131.38 | 85.9 | 125.7 | 114.3 ± 24.8 | |
| 8 | Untreated | A2922 | 7.88 | 55.36 | 55.4 | 39.5 ± 27.4 | 41.2 ± 2.4 |
| | | A2923 | 10.11 | 62.43 | 56.1 | 42.9 ± 28.6 | |

TABLE 5
Viral Genome Copy Number Quantification of FIG. 19.

| | | | Viral | | | Group |
|---|---|---|---|---|---|---|
| Group | Virus name | Mouse | Genomes/cell | | | mean ± |
| (male) | (code) | code | Exp 1 | Exp 2 | Mean | SD |
| 1 | OTC-wt | A2901 | 20.7 | 18.6 | 19.6 | 16.9 ± 3.3 |
| | (1668) | A2902 | 24.5 | 11.4 | 17.9 | |
| | | A2903 | 9.7 | 16.9 | 13.3 | |
| 2 | OT-C01 | A2904 | 39.6 | 16.9 | 28.2 | 28.7 ± 1.6 |
| | (1669) | A2905 | 25.1 | 29.6 | 27.4 | |
| | | A2906 | 40.9 | 20.1 | 30.5 | |
| 3 | OTC-C02 | A2907 | 16.7 | 13.8 | 15.2 | 17.0 ± 10.5 |
| | (1692) | A2908 | 8.8 | 6.2 | 7.5 | |
| | | A2909 | 29.4 | 27.1 | 28.2 | |
| 4 | OTC-C03 | A2910 | 16.6 | 18.5 | 17.5 | 14.8 ± 3.2 |
| | (1678) | A2911 | 13.4 | 9.0 | 11.2 | |
| | | A2912 | 20.0 | 11.3 | 15.6 | |
| 5 | OTC-C06 | A2913 | 56.7 | 30.9 | 43.8 | 20.2 ± 20.5 |
| | (1679) | A2914 | 11.0 | 9.0 | 10.0 | |
| | | A2915 | 7.6 | 6.1 | 6.8 | |
| 6 | OTC-C07 | A2916 | 39.5 | 43.0 | 41.3 | 53.3 ± 31.6 |
| | (1680) | A2917 | 28.6 | 30.3 | 29.5 | |
| | | A2918 | 88.6 | 89.7 | 89.1 | |
| 7 | OTC-C09 | A2919 | 52.4 | 45.3 | 48.8 | 56.4 ± 8.3 |
| | (1681) | A2920 | 64.3 | 46.0 | 55.1 | |
| | | A2921 | 75.4 | 55.0 | 65.2 | |

TABLE 6
Western Blot Quantification of FIG. 20.

| | | | Band intensity | | | |
|---|---|---|---|---|---|---|
| | Virus | | OTC/Tubulin | Fold | | |
| Group | name | Mouse | (normalized | change | | Standard |
| (male) | (code) | code | on Vg) | OTC-wt | Mean | deviation |
| 1 | OTC-wt | A2901 | 0.48 | 0.67 | 100 | 43 |
| | (1668) | A2902 | 0.61 | 0.85 | | |
| | | A2903 | 1.07 | 1.48 | | |
| 2 | OT-C01 | A2904 | 0.99 | 1.37 | 125 | 12 |
| | (1669) | A2905 | 0.90 | 1.24 | | |
| | | A2906 | 0.82 | 1.13 | | |
| 4 | OTC- | A2910 | 1.34 | 1.85 | 254 | 61 |
| | C03 | A2911 | 2.04 | 2.81 | | |
| | (1678) | A2912 | 2.15 | 2.97 | | |
| 5 | OTC- | A2913 | 0.71 | 0.97 | 365 | 243 |
| | C06 | A2914 | 3.09 | 4.27 | | |
| | (1679) | A2915 | 4.13 | 5.71 | | |

TABLE 7
Western Blot Quantification of FIG. 21.

| | | | Band intensity | Fold | | |
|---|---|---|---|---|---|---|
| | Virus | | OTC/Tubulin | change | | |
| Group | name | Mouse | (normalized on | OTC- | | Standard |
| (male) | (code) | code | Vg) | wt | Mean | deviation |
| 9 | OTC-wt | A3215 | 0.30 | 96% | 100% | 39 |
| | (1668) | A3216 | 0.19 | 63% | | |
| | | A3217 | 0.43 | 141% | | |
| 10 | OT-C01 | A3218 | 0.29 | 96% | 131% | 31 |
| | (1669) | A3219 | 0.45 | 148% | | |
| | | A3220 | 0.46 | 150% | | |
| 11 | OTC- | A3221 | 0.41 | 133% | 94% | 35 |
| | C02 | A3222 | 0.20 | 66% | | |
| | (1692) | A3223 | 0.25 | 81% | | |
| 12 | OTC- | A3224 | 0.26 | 86% | 105% | 41 |
| | C03 | A3225 | 0.46 | 151% | | |
| | (1678) | A3226 | 0.23 | 76% | | |
| 9 | OTC-wt | A3215 | 0.36 | 100% | 100% | 18 |
| | (1668) | A3216 | 0.30 | 82% | | |
| | | A3217 | 0.43 | 118% | | |
| 13 | OTC- | A3227 | 0.64 | 175% | 222% | 63 |
| 13 | C06 | A3228 | 0.72 | 196% | | |
| | (1679) | A3229 | 1.07 | 293% | | |
| 14 | OTC- | A3230 | 0.15 | 42% | 58% | 17 |
| | C07 | A3231 | 0.28 | 76% | | |
| | (1680) | A3232 | 0.20 | 55% | | |
| 15 | OTC- | A3233 | 0.25 | 69% | 146% | 108 |
| | C09 | A3234 | 0.81 | 223% | | |
| | (1681) | A3235 | | | | |

TABLE 8
OTC Catalytic Activity Quantification of FIG. 21.

| | | | OTC activity | | | | |
|---|---|---|---|---|---|---|---|
| | | | (μmol citrulline/μg | | | | |
| Group | Virus | Mouse | lysate/30 minutes) | | | | |
| (male) | name (code) | code | Exp. 1 | Exp. 1 | Exp. 3 | Mean | Mean ± SD |
| 9 | OTC-wt | A3215 | 128.7 | 143.0 | 92.9 | 121.6 | 94.1 ± 27.4 |
| | (1668) | A3216 | 113.4 | 101.1 | 67.3 | 93.9 | |
| | | A3217 | 85.9 | 21.0 | 93.7 | 66.9 | |
| 10 | OT-C01 | A3218 | 67.7 | 311.0 | 114.8 | 164.5 | 138.8 ± 29 |
| | (1669) | A3219 | 94.6 | 95.1 | 133.2 | 107.6 | |
| | | A3220 | 113.9 | 157.5 | 161.9 | 144.4 | |
| 11 | OTC- | A3221 | 69.0 | 53.0 | 70.8 | 64.3 | 74.2 ± 30 |
| | C02 | A3222 | 49.2 | 52.9 | 49.2 | 50.4 | |
| | (1692) | A3223 | 93.5 | 130.0 | 100.2 | 107.9 | |
| 12 | OTC- | A3224 | 122.5 | 84.3 | 150.4 | 119.1 | 123.1 ± 15.2 |
| | C03 | A3225 | 105.5 | 92.4 | 133.0 | 110.3 | |
| | (1678) | A3226 | 85.7 | 181.3 | 152.7 | 139.9 | |
| 13 | OTC- | A3227 | 164.0 | 93.2 | 197.6 | 151.6 | 305.5 ± 198.6 |
| | C06 | A3228 | 232.3 | 226.9 | 246.7 | 235.3 | |
| | (1679) | A3229 | 491.3 | 596.9 | 500.8 | 529.7 | |
| 14 | OTC- | A3230 | 54.7 | 41.4 | 95.8 | 64.0 | 93.9 ± 30.2 |
| | C07 | A3231 | 116.1 | 62.4 | 101.7 | 93.4 | |
| | (1680) | A3232 | 85.5 | 162.4 | 125.1 | 124.4 | |
| 15 | OTC- | A3233 | 129.1 | 143.1 | 104.2 | 125.5 | 108 ± 24.9 |
| | C09 | A3234 | 153.1 | 31.4 | 86.5 | 90.3 | |
| | (1681) | A3235 | | | | | |
| 16 | Untreated | A3236 | 55.1 | 88.4 | 57.5 | 67.0 | 62.9 ± 6.8 |
| | | A3237 | 55.8 | 62.3 | 46.9 | 55.0 | |
| | | A3238 | 55.6 | 91.2 | 53.4 | 66.7 | |

TABLE 9
Viral Genome Copy Number Quantification of FIG. 21.

| | Virus | | Viral | | | Group |
|---|---|---|---|---|---|---|
| Group | name | Mouse | Genomes/cell | | | mean ± |
| (male) | (code) | code | Exp. 1 | Exp. 2 | Mean | SD |
| 9 | OTC-wt | A3215 | 1.61 | 1.85 | 1.73 | 2.7 ± 1.1 |
| | (1668) | A3216 | 1.98 | 2.94 | 2.46 | |
| | | A3217 | 3.15 | 4.68 | 3.92 | |
| 10 | OT-C01 | A3218 | 3.58 | 3.60 | 3.59 | 2.3 ± 1.4 |
| | (1669) | A3219 | 0.73 | 0.94 | 0.84 | |
| | | A3220 | 2.21 | 2.84 | 2.52 | |
| 11 | OTC-C02 | A3221 | 2.16 | 2.86 | 2.51 | 5.4 ± 2.5 |
| | (1692) | A3222 | 6.34 | 7.78 | 7.06 | |
| | | A3223 | 5.23 | 8.13 | 6.68 | |
| 12 | OTC-C03 | A3224 | 6.92 | 12.57 | 9.74 | 7.6 ± 3.4 |
| | (1678) | A3225 | 3.21 | 4.14 | 3.67 | |
| | | A3226 | 9.13 | 9.75 | 9.44 | |
| 13 | OTC-C06 | A3227 | 1.91 | 2.03 | 1.97 | 1.7 ± 1.2 |
| | (1679) | A3228 | 2.02 | 3.45 | 2.73 | |
| | | A3229 | 0.24 | 0.41 | 0.32 | |
| 14 | OTC-C07 | A3230 | 14.28 | 20.16 | 17.22 | 11.8 ± 5.4 |
| | (1680) | A3231 | 5.16 | 7.56 | 6.36 | |
| | | A3232 | 9.27 | 14.12 | 11.69 | |
| 15 | OTC-C09 | A3233 | 5.97 | 6.40 | 6.18 | 2.9 ± 3.1 |
| | (1681) | A3234 | 1.81 | 2.86 | 2.34 | |
| | | A3235 | 0.09 | 0.21 | 0.15 | |

TABLE 10
Western Blot Quantification of FIG. 22.

| | | | Band | | | |
|---|---|---|---|---|---|---|
| | | | intensity | | | |
| | Virus | | OTC/Tubulin | Fold | | |
| Group | name | Mouse | (normalized | change | | Standard |
| female | (code) | code | on Vg) | OTC-wt | Mean | deviation |
| 1 | OTC- | A2929 | 0.11 | 195% | 100% | 86 |
| | wt | A2930 | 0.01 | 27% | | |
| | (1668) | A2931 | 0.04 | 78% | | |
| 2 | OT- | A2932 | 0.10 | 193% | 191% | 112 |
| | C01 | A2933 | 0.04 | 77% | | |
| | (1669) | A2934 | 0.16 | 302% | | |
| 3 | OTC- | A2935 | 0.08 | 142% | 122% | 51 |
| | C02 | A2936 | 0.03 | 64% | | |
| | (1692) | A2937 | 0.09 | 160% | | |
| 4 | OTC- | A2938 | 0.13 | 242% | 190% | 75 |
| | C03 | A2939 | 0.07 | 137% | | |
| | (1678) | A2940 | | | | |
| 1 | OTC- | A2929 | 0.11 | 159% | 100% | 59 |
| | wt | A2930 | 0.03 | 41% | | |
| | (1668) | A2931 | 0.07 | 100% | | |
| 5 | OTC- | A2941 | 0.10 | 145% | 103% | 37 |
| | C06 | A2942 | 0.06 | 90% | | |
| | (1679) | A2943 | 0.05 | 75% | | |
| 6 | OTC- | A2944 | 0.26 | 375% | 261% | 100 |
| | C07 | A2945 | 0.15 | 213% | | |
| | (1680) | A2946 | 0.13 | 193% | | |
| 7 | OTC- | A2947 | 0.05 | 71% | 107% | 54 |
| | C09 | 2948 | 0.12 | 169% | | |
| | (1681) | A2949 | 0.06 | 81% | | |

TABLE 11
OTC Catalytic Activity Quantification of FIG. 22.

| | | | OTC activity (μmol | | | | |
|---|---|---|---|---|---|---|---|
| | Virus | | citrulline/μg lysate/30 | | | | Group |
| Group | name | Mouse | minutes) | | | Individual | Mean ± |
| Female | (code) | code | Exp. 1 | Exp. 2 | Exp. 3 | Mean ± SD | SD |
| 1 | OTC-wt | A2929 | 29.39 | 28.1 | 17.2 | 24.9 ± 6.7 | 16.3 ± 8.2 |
| | (1668) | A2930 | 8.41 | 3.4 | 13.7 | 8.5 ± 5.2 | |
| | | A2931 | 14.29 | 9.9 | 22.2 | 15.5 ± 6.2 | |
| 2 | OT-C01 | A2932 | 49.04 | 29.2 | 48.2 | 42.1 ± 11.2 | 33.6 ± 21 |
| | (1669) | A2933 | 11.20 | 11.3 | 6.8 | 9.8 ± 2.6 | |
| | | A2934 | 60.23 | 56.1 | 30.7 | 49.0 ± 16.0 | |
| 3 | OTC-C02 | A2935 | 38.60 | 20.6 | 32.7 | 30.6 ± 9.2 | 26.4 ± 10.5 |
| | (1692) | A2936 | 14.27 | 9.2 | 20.0 | 14.5 ± 5.4 | |
| | | A2937 | 36.94 | 26.4 | 39.0 | 34.1 ± 6.8 | |
| 4 | OTC-C03 | A2938 | 59.79 | 24.4 | 43.4 | 42.5 ± 17.7 | 35.3 ± 10.2 |
| | (1678) | A2939 | 41.05 | 19.7 | 23.7 | 28.2 ± 11.3 | |
| | | A2940 | | | | | |
| 5 | OTC-C06 | A2941 | 24.92 | 19.9 | 20.7 | 21.8 ± 2.7 | 16.3 ± 4.8 |
| | (1679) | A2942 | 14.18 | 11.9 | 14.8 | 13.6 ± 1.5 | |
| | | A2943 | 12.54 | 12.1 | 15.9 | 13.5 ± 2.1 | |
| 6 | OTC-C07 | A2944 | 76.87 | 32.5 | 93.2 | 67.5 ± 31.4 | 41.8 ± 23.3 |
| | (1680) | A2945 | 40.60 | 30.1 | 36.7 | 35.8 ± 5.3 | |
| | | A2946 | 23.97 | 21.8 | 20.2 | 22.0 ± 1.9 | |
| 7 | OTC-C09 | A2947 | 16.72 | 8.4 | 8.6 | 11.2 ± 4.7 | 28.1 ± 30.7 |
| | (1681) | A2948 | 79.82 | 38.3 | 72.3 | 63.5 ± 22.1 | |
| | | A2949 | 23.23 | 4.8 | 0.5 | 9.5 ± 12.1 | |
| 8 | untreated | A2950 | 29.39 | 7.8 | 13.3 | 16.8 ± 11.2 | 10.8 ± 3.7 |
| | | A2951 | 8.41 | 13.7 | 4.2 | 8.8 ± 4.8 | |

TABLE 12
Viral Genome Copy Number Quantification of FIG. 22.

| | Virus | | Viral | | | |
|---|---|---|---|---|---|---|
| Group | name | Mouse | Genomes/cell | | | Group mean ± |
| female | (code) | code | Exp. 1 | Exp. 2 | Mean | SD |
| 1 | OTC-wt | A2929 | 4.2 | 4.39 | 4.3 | 17.2 ± 15.3 |
| | (1668) | A2930 | 31.4 | 36.85 | 34.1 | |
| | | A2931 | 12.6 | 13.90 | 13.3 | |
| 2 | OT-C01 | A2932 | 6.6 | 9.82 | 8.2 | 12 ± 7.9 |
| | (1669) | A2933 | 22.0 | 20.05 | 21.0 | |
| | | A2934 | 6.9 | 6.49 | 6.7 | |
| 3 | OTC-C02 | A2935 | 15.5 | 14.42 | 15.0 | 21.3 ± 15.3 |
| | (1692) | A2936 | 27.4 | 50.22 | 38.8 | |
| | | A2937 | 9.0 | 11.37 | 10.2 | |
| 4 | OTC-C03 | A2938 | 10.1 | 9.10 | 9.6 | 8.5 ± 7.7 |
| | (1678) | A2939 | 17.3 | 13.93 | 15.6 | |
| | | A2940 | 0.2 | 0.59 | 0.4 | |
| 5 | OTC-C06 | A2941 | 28.3 | 21.11 | 24.7 | 31.1 ± 7.4 |
| | (1679) | A2942 | 36.7 | 41.63 | 39.2 | |
| | | A2943 | 32.9 | 26.11 | 29.5 | |
| 6 | OTC-C07 | A2944 | 6.2 | 9.74 | 7.9 | 16.8 ± 8.7 |
| | (1680) | A2945 | 15.9 | 18.54 | 17.2 | |
| | | A2946 | 27.6 | 23.05 | 25.3 | |
| 7 | OTC-C09 | A2947 | 45.4 | 36.86 | 41.1 | 22.2 ± 17.3 |
| | (1681) | A2948 | 7.2 | 7.39 | 7.3 | |
| | | A2949 | 17.3 | 18.89 | 18.1 | |

TABLE 13
Experiment 1 - High dose - Males.

| Group | Virus name | Viral | Mouse | | Mouse | Vg/ | | |
|---|---|---|---|---|---|---|---|---|
| (male) | (code) | batch | code | Vg/Kg | weight (gr) | mouse | μl virus | PBS |
| 1 | OTC-wt | First | A2901 | 5.0E12 | 23.5 | 1.15E11 | 90.4 | |
| | (1668) | prep | A2902 | 5.0E12 | 23.6 | 1.18E11 | 90.8 | |
| | | | A2903 | 5.0E12 | 25.4 | 1.27E11 | 97.7 | |
| 2 | OT-C01 | First | A2904 | 5.0E12 | 22.4 | 1.12E11 | 70 | 20 |
| | (1669) | prep | A2904 | 5.0E12 | 22.4 | 1.12E11 | 70 | 20 |
| | | | A2906 | 5.0E12 | 22.5 | 1.12E11 | 70.3 | 19.7 |
| 3 | OTC-C02 | First | A2907 | 5.0E12 | 26 | 1.13E11 | 17.8 | 72.2 |
| | (1692) | prep | A2908 | 5.0E12 | 22.3 | 1.15E11 | 15.3 | 74.7 |
| | | | A2909 | 5.0E12 | 21 | 1.05E11 | 14.4 | 75.6 |
| 4 | OTC-C03 | First | A2910 | 5.0E12 | 26.3 | 1.35E11 | 18.8 | 71.2 |
| | (1678) | prep | A2911 | 5.0E12 | 26.1 | 1.35E11 | 18.6 | 71.4 |
| | | | A2912 | 5.0E12 | 22.9 | 1.35E11 | 16.4 | 73.6 |
| 5 | OTC-C06 | First | A2913 | 5.0E12 | 23 | 1.15E11 | 31.9 | 58 |
| | (1679) | prep | A2914 | 5.0E12 | 26.8 | 1.34E11 | 37.2 | 52.8 |
| | | | A2915 | 5.0E12 | 22.3 | 1.15E11 | 31 | 59 |
| 6 | OTC-C07 | First | A2916 | 5.0E12 | 25 | 1.25E11 | 11.4 | 78.6 |
| | (1680) | prep | A2917 | 5.0E12 | 23.8 | 1.19E11 | 10.8 | 79.2 |
| | | | A2918 | 5.0E12 | 25.8 | 1.29E11 | 11.7 | 78.3 |
| 7 | OTC-C09 | First | A2919 | 5.0E12 | 25.3 | 1.25E11 | 17.1 | 72.9 |
| | (1681) | prep | A2920 | 5.0E12 | 25.8 | 1.29E11 | 17.4 | 72.6 |
| | | | A2921 | 5.0E12 | 22.1 | 1.15E11 | 14.9 | 75 |
| 8 | No treatment | | A2922 | | 23.4 | | | 90 |
| | | | A2923 | | 23.8 | | | 90 |
| | | | A2924 | | 20.6 | | | 90 |

TABLE 14
Experiment 1 - High dose - Females.

| Group | Virus name | Viral | Mouse | | Mouse | | | |
|---|---|---|---|---|---|---|---|---|
| (female) | (code) | batch | code | Vg/Kg | weight (gr) | Vg/mouse | μl virus | PBS |
| 1 | OTC-wt | First | A2929 | 5.0E12 | 18 | 9.0E10 | 69.2 | 20.7 |
| | (1668) | prep | A2930 | 5.0E12 | 16.7 | 8.35E10 | 64.2 | 25.8 |
| | | | A2931 | 5.0E12 | 18.8 | 9.4E10 | 72.3 | 17.7 |
| 2 | OT-C01 | First | A2932 | 5.0E12 | 19.3 | 9.65E10 | 60.3 | 30 |
| | (1669) | prep | A2933 | 5.0E12 | 16 | 8.0E10 | 50 | 40 |
| | | | A2934 | 5.0E12 | 16.5 | 8.25E10 | 51.6 | 38 |
| 3 | OTC-C02 | First | A2935 | 5.0E12 | 18 | 9E10 | 12.3 | 77.7 |
| | (1692) | prep | A2936 | 5.0E12 | 17.9 | 8.95E10 | 12.3 | 77.7 |
| | | | A2937 | 5.0E12 | 17 | 8.5E10 | 11.6 | 78 |
| 4 | OTC-C03 | First | A2938 | 5.0E12 | 18.1 | 9.05E10 | 12.9 | 77 |
| | (1678) | prep | A2939 | 5.0E12 | 17.4 | 8.7E10 | 12.4 | 77.5 |
| | | | A2940 | 5.0E12 | 10.2 | 5.1E10 | 7.3 | 83 |
| 5 | OTC-C06 | First | A2941 | 5.0E12 | 17.2 | 8.6E10 | 23.9 | 66 |
| | (1679) | prep | A2942 | 5.0E12 | 18.4 | 9.2E10 | 25.6 | 64 |
| | | | A2943 | 5.0E12 | 19 | 9.5E10 | 26.4 | 64 |
| 6 | OTC-C07 | First | A2944 | 5.0E12 | 17.3 | 8.65E10 | 7.8 | 82 |
| | (1680) | prep | A2945 | 5.0E12 | 17.1 | 8.55E10 | 7.7 | 82 |
| | | | A2946 | 5.0E12 | 15.9 | 7.95E10 | 7.2 | 78 |
| 7 | OTC-C09 | First | A2947 | 5.0E12 | 17 | 8.5E10 | 11.5 | 78 |
| | (1681) | prep | A2948 | 5.0E12 | 18.1 | 9.05E10 | 12.2 | 72.6 |
| | | | A2949 | 5.0E12 | 17.7 | 8.85E10 | 12 | 75 |
| 8 | No treatment | | A2950 | | 17.1 | | | 90 |
| | | | A2951 | | 16.3 | | | 90 |
| | | | A2952 | | 18.7 | | | 90 |

TABLE 15
Experiment 1 - Low dose - Females.

| | | | | | Mouse | | | |
|---|---|---|---|---|---|---|---|---|
| Group | Virus name | Viral | Mouse | | weight | | μl | |
| (male) | (code) | batch | code | Vg/Kg | (gr) | Vg/mouse | virus | PBS |
| 9 | OTC-wt | First | A3215 | 1.25E+12 | 31 | 3.88E+11 | 29.8 | 60.2 |
| | (1668) | prep | A3216 | 1.25E+12 | 30 | 3.7E+11 | 28.8 | 61.2 |
| | | | A3217 | 1.25E+12 | 29 | 3.6E+11 | 27.9 | 62.1 |
| 10 | OT-C01 | First | A3218 | 1.25E+12 | 28 | 3.5E+11 | 21.9 | 68.1 |
| | (1669) | prep | A3219 | 1.25E+12 | 31 | 3.8E+11 | 24.2 | 65.8 |
| | | | A3220 | 1.25E+12 | 30 | 3.75E+11 | 23.4 | 66.6 |
| 11 | OTC-C02 | First | A3221 | 1.25E+12 | 31 | 3.8E+11 | 5.3 | 84.7 |
| | (1692) | prep | A3222 | 1.25E+12 | 32 | 4.0E+11 | 5.5 | 84.5 |
| | | | A3223 | 1.25E+12 | 30.5 | 3.8E+11 | 5.2 | 84.8 |
| 12 | OTC-C03 | First | A3224 | 1.25E+12 | 28 | 3.5E+11 | 5 | 85 |
| | (1678) | prep | A3225 | 1.25E+12 | 27 | 3.4E+11 | 4.8 | 85.2 |
| | | | A3226 | 1.25E+12 | 30 | 3.7E+11 | 5.4 | 84.6 |
| 13 | OTC-C06 | First | A3227 | 1.25E+12 | 30 | 3.7E+11 | 10.4 | 79.6 |
| | (1679) | prep | A3228 | 1.25E+12 | 29.5 | 3.7E+11 | 10.2 | 79.8 |
| | | | A3229 | 1.25E+12 | 30 | 3.75E+11 | 10.4 | 79.6 |
| 14 | OTC-C07 | First | A3230 | 1.25E+12 | 29 | 3.7E+11 | 3.3 | 86.7 |
| | (1680) | prep | A3231 | 1.25E+12 | 32 | 4.0E+11 | 3.6 | 86.4 |
| | | | A3232 | 1.25E+12 | 30 | 3.75E+11 | 3.4 | 86.6 |
| 15 | OTC-C09 | First | A3233 | 1.25E+12 | 30 | 3.75E+11 | 5 | 84.9 |
| | (1681) | prep | A3234 | 1.25E+12 | 30 | 3.75E+11 | 5 | 84.9 |
| | | | A3235 | 1.25E+12 | 29 | 3.63E+11 | 4.9 | 85.1 |
| 16 | No treatment | | A3236 | 1.25E+12 | 28 | | | 90 |
| | | | A3237 | 1.25E+12 | 32 | | | 90 |
| | | | A3238 | 1.25E+12 | 29 | | | 90 |

TABLE 16
Experimental Groups and Doses-Second Preparation.

| | Virus | | | | Mouse | | | |
|---|---|---|---|---|---|---|---|---|
| Group | name | Virus | Mouse | | weight | | μl | |
| (male) | (code) | batch | code | Vg/Kg | (gr) | Vg/mouse | virus | PBS |
| 17 | OTC-wt | 2nd | A3250 | 1.25E+12 | 29 | 3.63E+10 | 3 | 47 |
| | (16137) | prep | A3251 | 1.25E+12 | 32 | 4.00E+10 | 3.3 | 46.7 |
| | | | A3252 | 1.25E+12 | 31 | 3.88E+10 | 3.2 | 46.8 |
| 18 | OT-C01 | 2nd | A3253 | 1.25E+12 | 34 | 4.25E+10 | 4.4 | 45.6 |
| | (16138) | prep | A3254 | 1.25E+12 | 31.5 | 3.94E+10 | 4.1 | 45.9 |
| | | | A3255 | 1.25E+12 | 34 | 4.25E+10 | 4.4 | 45.6 |
| 19 | OTC- | 2nd | A3256 | 1.25E+12 | 25 | 3.13E+10 | 4.7 | 45.3 |
| | C03 | prep | A3257 | 1.25E+12 | 30 | 3.75E+10 | 5.7 | 44.3 |
| | (16139) | | A3258 | 1.25E+12 | 30 | 3.75E+10 | 5.7 | 44.3 |
| 20 | OTC- | 2nd | A3259 | 1.25E+12 | 40 | 5.00E+10 | 6.9 | 43.1 |
| | C06 | prep | A3260 | 1.25E+12 | 30 | 3.75E+10 | 5.2 | 44.8 |
| | (16140) | | A3261 | 1.25E+12 | 30 | 3.75E+10 | 5.2 | 44.8 |
| 21 | No | | A3262 | 1.25E+12 | 36 | | | 50 |
| | treatment | | A3263 | 1.25E+12 | 36 | | | 50 |

TABLE 17
Western Blot Quantification of FIG. 24.

| | | | Band intensity | | | |
|---|---|---|---|---|---|---|
| | Virus | | OTC/Tubulin | Fold | | |
| Group | name | Mouse | (normalized on | change | | Standard |
| (male) | (code) | code | Vg) | OTC-wt | Mean | deviation |
| 17 | OTC- | A3250 | 0.07 | 87% | 100% | 31% |
| | wt | A3251 | 0.06 | 79% | | |
| | (16137) | A3252 | 0.10 | 135% | | |
| 18 | OT- | A3253 | 0.19 | 253% | 253% | 78% |
| | C01 | A3254 | 0.16 | 206% | | |
| | (16138) | A3255 | 0.28 | 359% | | |
| 19 | OTC- | A3256 | 0.64 | 836% | 459% | 329% |
| | C03 | A3257 | 0.24 | 314% | | |
| | (16139) | A3258 | 0.17 | 227% | | |
| 20 | OTC- | A3259 | 0.27 | 345% | 451% | 150% |
| | C06 | A3260 | 0.43 | 557% | | |
| | (16140) | A3261 | 0.04 | | | |

TABLE 18
OTC Catalytic Activity Quantification of FIG. 24.

| | | | OTC | | | |
|---|---|---|---|---|---|---|
| | | | ACTIVITY (μmol | | | |
| | Virus | | citrulline/μg | | Fold | |
| Group | name | Mouse | lysate/30 minutes) | | change/ | Mean ± |
| (male) | (code) | code | Exp. 1 | Exp. 2 | wtOTC/vg | SD |
| 17 | OTC- | A3250 | 44.1 | 43.4 | 68% | 100 ± 33 |
| | wt | A3251 | 28.8 | 31.5 | 99% | |
| | (16137) | A3252 | 52.1 | 44.2 | 133% | |
| 18 | OT- | A3253 | 105.6 | 46.4 | 115% | 86 ± 27 |
| | C01 | A3254 | 95.4 | 38.1 | 82% | |
| | (16138) | A3255 | 88.8 | 32.1 | 62% | |
| 19 | OTC- | A3256 | | | | 229 ± 67 |
| | C03 | A3257 | 158.2 | 58.6 | 276% | |
| | (16139) | A3258 | 132.7 | 58.5 | 181% | |
| 20 | OTC- | A3259 | 96.4 | 34.7 | 74% | 204 ± 145 |
| | C06 | A3260 | 214.6 | 76.2 | 360% | |
| | (16140) | A3261 | 162.2 | 52.8 | 179% | |
| 21 | No | A3262 | 28.9 | 16.4 | | |
| | treatment | A3263 | 32.7 | 24.4 | | |

TABLE 19
Viral Genome Copy Number Quantification of FIG. 24.

| | | | | | | Group |
|---|---|---|---|---|---|---|
| Group | Virus name | Mouse | Viral Genomes/cell | | | mean ± |
| (male) | (code) | code | Exp. 1 | Exp. 2 | Mean | SD |
| 17 | OTC-wt | A3250 | 10.2 | 10.71 | 10.5 | 7.1 ± 3.0 |
| | (16137) | A3251 | 5.7 | 4.36 | 5.0 | |
| | | A3252 | 5.8 | 6.01 | 5.9 | |
| 18 | OT-C01 | A3253 | 11.7 | 9.99 | 10.8 | 13.4 ± 2.6 |
| | (16138) | A3254 | 14.1 | 12.54 | 13.3 | |
| | | A3255 | 16.3 | 15.81 | 16.0 | |
| 19 | OTC-C03 | A3256 | 1.4 | 1.27 | 1.4 | 5.5 ± 3.7 |
| | (16139) | A3257 | 6.9 | 6.03 | 6.4 | |
| | | A3258 | 9.4 | 7.85 | 8.6 | |
| 20 | OTC-C06 | A3259 | 16.1 | 12.84 | 14.5 | 10.3 ± 4.0 |
| | (16140) | A3260 | 7.4 | 5.77 | 6.6 | |
| | | A3261 | 10.6 | 9.08 | 9.8 | |

TABLE 20
Experimental Groups and Doses - CO1, CO3, CO21.

| | Virus | | | | Mouse | | | |
|---|---|---|---|---|---|---|---|---|
| Group | name | Virus | Mouse | | weight | Vg/ | μl | |
| (male) | (code) | batch | code | Vg/Kg | (g) | mouse | virus | PBS |
| 22 | OTCwt | 2nd | A4723 | 1.25E+12 | 25 | 3.13E+10 | 2.31 | 47.69 |
| | (16137) | prep | A4724 | 1.25E+12 | 27 | 3.38E+10 | 2.50 | 47.50 |
| | | | A4709 | 1.25E+12 | 29 | 3.63E+10 | 2.69 | 47.31 |
| | | | A4710 | 1.25E+12 | 28 | 3.50E+10 | 2.59 | 47.41 |
| 23 | OTC1 | 2nd | A4711 | 1.25E+12 | 28 | 3.50E+10 | 3.10 | 46.90 |
| | (16138) | prep | A4712 | 1.25E+12 | 27 | 3.38E+10 | 2.99 | 47.01 |
| | | | A4713 | 1.25E+12 | 28 | 3.50E+10 | 3.10 | 46.90 |
| | | | A4722 | 1.25E+12 | 25 | 3.13E+10 | 2.77 | 47.23 |
| 24 | OTC 3 | 2nd | A4715 | 1.25E+12 | 30 | 3.75E+10 | 5.24 | 44.76 |
| | (16139) | prep | A4716 | 1.25E+12 | 25 | 3.13E+10 | 4.37 | 45.63 |
| | | | A4717 | 1.25E+12 | 26 | 3.25E+10 | 4.55 | 45.45 |
| | | | A4720 | 1.25E+12 | 23 | 2.88E+10 | 4.02 | 45.98 |
| 25 | OTC | Third | A4719 | 1.25E+12 | 26 | 3.25E+10 | 19.12 | 30.88 |
| | 21 | prep | A4721 | 1.25E+12 | 25 | 3.13E+10 | 18.38 | 31.62 |
| | (17115) | | A4718 | 1.25E+12 | 25 | 3.13E+10 | 18.38 | 31.62 |
| | | | A4708 | 1.25E+12 | 30 | 3.75E+10 | 22.06 | 27.94 |
| 26 | No | | A4726 | | | | | |
| | treatment | | A4727 | | | | | |

TABLE 21
Western Blot Quantification of FIG. 25.

| | | | Band | | | |
|---|---|---|---|---|---|---|
| | | | intensity OTC/ | Fold | | |
| | Virus | | TUBULIN | change | | |
| Group | name | Mouse | (normalized | OTC- | | Standard |
| (male) | (code) | code | on Vg) | wt | Mean | deviation |
| 22 | OTCwt | A4723 | 0.016 | 103% | 100% | 42 |
| | (16137) | A4724 | 0.021 | 140% | | |
| | | A4709 | 0.009 | 57% | | |
| 23 | OTC1 | A4711 | 0.045 | 296% | 345% | 60 |
| | (16138) | A4712 | 0.050 | 327% | | |
| | | A4713 | 0.063 | 412% | | |
| 24 | OTC 3 | A4715 | 0.051 | 330% | 359% | 75 |
| | (16139) | A4716 | 0.068 | 444% | | |
| | | A4717 | 0.046 | 303% | | |
| 25 | OTC 21 | A4719 | 0.078 | 505% | 561% | 56 |
| | (17115) | A4721 | 0.086 | 562% | | |
| | | A4718 | 0.095 | 616% | | |

TABLE 22
OTC Catalytic Activity Quantification of FIG. 25.

| | Virus | | | OD | Fold | |
|---|---|---|---|---|---|---|
| Group | name | Virus | | 490 nm/ | change/ | Mean ± |
| (male) | (code) | batch | Mouse code | vg | wtOTC | SD |
| 22 | OTCwt | Second | A4723 | 0.0153 | 129% | 100 ± 20 |
| | (16137) | prep | A4724 | 0.0112 | 94% | |
| | | | A4709 | 0.0102 | 86% | |
| | | | A4710 | 0.0106 | 90% | |
| 23 | OTC1 | Second | A4711 | 0.0343 | 290% | 341 ± 37 |
| | (16138) | Prep | A4712 | 0.0439 | 371% | |
| | | | A4713 | 0.0400 | 338% | |
| | | | A4722 | 0.0431 | 365% | |
| 24 | OTC 3 | Second | A4715 | 0.0537 | 454% | 477 ± 59 |
| | (16139) | Prep | A4716 | 0.0565 | 478% | |
| | | | A4717 | 0.0493 | 417% | |
| | | | A4720 | 0.0658 | 557% | |
| 25 | OTC 21 | Third | A4719 | 0.0733 | 620% | 618 ± 8 |
| | (17115) | prep | A4721 | 0.0721 | 610% | |
| | | | A4718 | 0.0742 | 627% | |
| | | | A4708 | 0.0727 | 615% | |

TABLE 23
Viral Genome Copy Number Quantification of FIG. 25.

| | | | | Viral | | | |
|---|---|---|---|---|---|---|---|
| | Virus | | | Genomes/cell | | | Group |
| Group | name | Virus | Mouse | Exp. | | | mean ± |
| (male) | (code) | batch | code | 1 | Exp. 2 | Mean | SD |
| 22 | OTCwt | Second | A4723 | 20.3 | 23.4 | 21.9 | 25 ± 7.8 |
| | (16137) | prep | A4724 | 16.0 | 15.4 | 15.7 | |
| | | | A4709 | 36.3 | 30.5 | 33.4 | |
| | | | A4710 | 31.1 | 27.0 | 29.1 | |
| 23 | OTC1 | Second | A4711 | 21.5 | 26.6 | 24.1 | 22.6 ± 3.8 |
| | (16138) | prep | A4712 | 26.3 | 28.2 | 27.2 | |
| | | | A4713 | 16.7 | 20.4 | 18.6 | |
| | | | A4722 | 20.0 | 21.1 | 20.5 | |
| 24 | OTC 3 | Second | A4715 | 31.1 | 31.1 | 31.1 | 23.9 ± 6.5 |
| | (16139) | prep | A4716 | 17.8 | 19.9 | 18.9 | |
| | | | A4717 | 23.5 | 31.6 | 27.6 | |
| | | | A4720 | 17.5 | 18.4 | 18.0 | |
| 25 | OTC | Third | A4719 | 13.7 | | | 18.1 ± 4.8 |
| | 21 | prep | A4721 | 24.6 | | | |
| | (17115) | | A4718 | 18.7 | | | |
| | | | A4708 | 15.4 | | | |

TABLE 24
Experimental Groups and Doses - OTC^{spf-ash} Pilot Study.

| | Virus | | | | Mouse | | | |
|---|---|---|---|---|---|---|---|---|
| Group | name | Virus | Mouse | Vg/ | weight | | | |
| (male) | (code) | batch | code | Kg | (gr) | Vg/mouse | μl virus | PBS |
| 27 | OTCwt | Second | A3633 | 5.0E+11 | 23.4 | 1.17E+10 | 9.00 | 41.00 |
| | (16137) | prep | A3635 | 5.0E+11 | 25.5 | 1.28E+10 | 9.81 | 40.19 |
| | | | A3636 | 5.0E+11 | 26.7 | 1.34E+10 | 10.27 | 39.73 |
| | | | A3637 | 5.0E+11 | 28.7 | 1.44E+10 | 11.04 | 38.96 |
| 28 | OTC1 | Second | A3638 | 5.0E+11 | 28 | 1.40E+10 | 8.75 | 41.25 |
| | (16138) | prep | A3640 | 5.0E+11 | 19.4 | 9.70E+09 | 6.06 | 43.94 |
| | | | A3641 | 5.0E+11 | 26.8 | 1.34E+10 | 8.37 | 41.63 |
| | | | A3642 | 5.0E+11 | 22 | 1.10E+10 | 6.87 | 43.13 |
| 29 | OTC 3 | Second | A3643 | 5.0E+11 | 25 | 1.25E+10 | 1.79 | 48.21 |
| | (16139) | prep | A3644 | 5.0E+11 | 23.3 | 1.17E+10 | 1.66 | 48.34 |
| | | | A3645 | 5.0E+11 | 15 | 7.50E+09 | 1.07 | 48.93 |
| | | | A3646 | 5.0E+11 | 17.1 | 8.55E+09 | 1.22 | 48.78 |
| 30 | OTC 6 | Second | A3647 | 5.0E+11 | 22.7 | 1.14E+10 | 3.15 | 46.85 |
| | (17115) | prep | A3648 | 5.0E+11 | 21.4 | 1.07E+10 | 2.97 | 47.03 |
| | | | A3657 | 5.0E+11 | 20.9 | 1.05E+10 | 2.90 | 47.10 |
| | | | A3658 | 5.0E+11 | 22.4 | 9.50E+09 | 2.64 | 47.36 |
| 31 | No | | A3649 | | | | | |
| | treatment | | A3650 | | | | | |

TABLE 25
Urinary orotic acid measurement in OTC^{spf-ash} male mice in FIG. 26.

| | | | μmol Orotic acid/mmol creatinine | | | | |
|---|---|---|---|---|---|---|---|
| | Virus | Mouse | | T2 | | | |
| Group | name (code) | code | T0 | weeks | T4 weeks | T6 weeks | T8 weeks |
| 27 | OTCwt | A3633 | 530.6 | −23.8 | 99.0 | 12.1 | 28.77 |
| | (16137) | A3635 | 1790.7 | 747.7 | 439.2 | 569.2 | 123.82 |
| | | A3636 | 84.6 | 62.8 | 94.4 | 328.8 | 87.54 |
| | | A3637 | 67.7 | 17.4 | 60.7 | 7.0 | 76.32 |
| 28 | OTC1 | A3638 | 346.6 | −12.6 | 44.4 | 11.2 | 43.25 |
| | (16138) | A3640 | 340.8 | 208.3 | 32.1 | 32.4 | 33.2 |
| | | A3641 | | | | | |
| | | A3642 | 240.8 | 6.0 | 215.1 | 5.0 | 28.81 |
| 29 | OTC 3 | A3643 | 687.4 | 118.2 | 226.2 | 173.4 | 76.66 |
| | (16139) | A3644 | 77.6 | 566.5 | 305.9 | 6 | 52.55 |
| | | A3645 | 1247.8 | 423.7 | 5.0 | 167.9 | 59.43 |
| | | A3646 | 492.7 | 18.6 | 21.0 | 53.2 | 56.2 |
| 30 | OTC 6 | A3647 | 273.4 | 519.6 | 183.2 | 134.7 | 173.62 |
| | (17115) | A3648 | 223.4 | 103.3 | 296.4 | 75.5 | 63.47 |
| | | A3657 | 657.9 | 425.8 | 76.1 | 18 | 69.75 |
| | | A3658 | 354.6 | 712.7 | 760.4 | 50.3 | 49.66 |
| 31 | untr | A3649 | 1200.0 | 523.8 | 757.4 | 573.5 | 481.42 |
| | | A3650 | 670.0 | 577.6 | 991.5 | 500 | 703.48 |

TABLE 26
Plasma ammonia levels in OTC^{spf-ash} male mice in FIG. 27.

| | Virus | | | | | |
|---|---|---|---|---|---|---|
| | name | Mouse | mmol NH₄ | | | |
| Group | (code) | code | T0 | | T4 weeks | |
| 27 | OTCwt | A3633 | 2.42 | 2.25 ± 0.21 | 0.79 | 0.72 ± 0.05 |
| | (16137) | A3635 | 2.03 | | 0.71 | |
| | | A3636 | 2.44 | | 0.71 | |
| | | A3637 | 2.1 | | 0.68 | |
| 28 | OTC1 | A3638 | 2.56 | 2.31 ± 0.24 | 1.25 | 1.02 ± 0.19 |
| | (16138) | A3640 | 2.01 | | 1.07 | |
| | | A3641 | 2.25 | | 0.79 | |
| | | A3642 | 2.41 | | 0.96 | |
| 29 | OTC 3 | A3643 | 3.07 | 2.92 ± 0.53 | 0.68 | 0.72 ± 0.11 |
| | (16139) | A3644 | 3.09 | | 0.82 | |
| | | A3645 | 3.37 | | 0.79 | |
| | | A3646 | 2.16 | | 0.57 | |
| 30 | OTC 6 | A3647 | 2.26 | 2.60 ± 0.60 | 0.75 | 0.74 ± 0.04 |
| | (17115) | A3648 | 1.93 | | 0.79 | |
| | | A3657 | 2.97 | | 0.71 | |
| | | A3658 | 3.22 | | 0.71 | |
| 31 | Untreated | | 3.26 | 2.93 ± 0.47 | 3.93 | 3.76 ± 0.24 |
| | | | 2.59 | | 3.59 | |
| | WT | | 1.82 | 1.84 ± 0.02 | 1.89 | 1.78 ± 0.30 |
| | | | 1.85 | | 1.46 | |

TABLE 27
Western Blot Quantification of FIG. 28.

| | | | | | Band | | |
|---|---|---|---|---|---|---|---|
| | | | | | intensity | | |
| | | | | | OTC/ | | |
| | | | | | Tubulin | Fold | |
| | Virus | | | | (normalized | change | |
| Group | name | Virus | Mouse | | on | vs.OTC- | Mean ± |
| (male) | (code) | batch | code | Vg/Kg | Vg) | wt | SD |
| 27 | OTCwt | Second | A3633 | 5.0E+11 | 1.813 | 94% | 100 ± 28 |
| | (16137) | prep | A3635 | 5.0E+11 | 2.041 | 105% | |
| | | | A3636 | 5.0E+11 | 2.598 | 134% | |
| | | | A3637 | 5.0E+11 | 1.287 | 67% | |
| 28 | OTC1 | Second | A3638 | 5.0E+11 | 5.175 | 267% | 191 ± 117 |
| | (16138) | prep | A3640 | 5.0E+11 | 4.822 | 249% | |
| | | | A3641 | 5.0E+11 | | | |
| | | | A3642 | 5.0E+11 | 1.093 | 57% | |
| 29 | OTC 3 | Second | A3643 | 5.0E+11 | 7.186 | 349% | 357 ± 143 |
| | (16139) | prep | A3644 | 5.0E+11 | 5.176 | 264% | |
| | | | A3645 | 5.0E+11 | 14.581 | 561% | |
| | | | A3646 | 5.0E+11 | 3.411 | 254% | |
| 27 | OTCwt | Second | A3633 | 5.0E+11 | 2.244 | 105% | 100 ± 26 |
| | (16137) | prep | A3635 | 5.0E+11 | 2.237 | 105% | |
| | | | A3636 | 5.0E+11 | 2.697 | 126% | |
| | | | A3637 | 5.0E+11 | 1.374 | 64% | |
| 29 | OTC 3 | Second | A3643 | 5.0E+11 | 7.186 | 336% | 355 ± 230 |
| | (16139) | prep | A3644 | 5.0E+11 | 5.176 | 242% | |
| | | | A3645 | 5.0E+11 | 14.581 | 682% | |
| | | | A3646 | 5.0E+11 | 3.411 | 160% | |
| 30 | OTC6 | Second | A3647 | 5.0E+11 | 19.657 | 919% | 512 ± 298 |
| | (17115) | prep | A3648 | 5.0E+11 | 5.335 | 250% | |
| | | | A3657 | 5.0E+11 | 7.201 | 337% | |
| | | | A3658 | 5.0E+11 | 11.615 | 543% | |

TABLE 28
OTC Catalytic Activity Quantification of FIG. 28.

| | | | | | | OD | | |
|---|---|---|---|---|---|---|---|---|
| | Virus | | | | | 490 | Fold | |
| Group | name | Virus | Mouse | μmol | Mean ± | nm/ | change/ | Mean ± |
| (male) | (code) | batch | code | citrullline | SD | vg | wtOTC | SD |
| 27 | OTCwt | Second | A4723 | 85.9 | 37.2 ± 32.7 | 0.03 | 139% | 100 ± 20 |
| | (16137) | prep | A4724 | 18.2 | | 0.02 | 70% | |
| | | | A4709 | 17.9 | | 0.03 | 130% | |
| | | | A4710 | 26.6 | | 0.01 | 61% | |
| 28 | OTC1 | Second | A4711 | 62.8 | 94.8 ± 107.2 | 0.09 | 372% | |
| | (16138) | prep | A4712 | 52.0 | | 0.05 | 191% | 236 ± 37 |
| | | | A4713 | 12.2 | | 0.08 | | |
| | | | A4722 | 252.3 | | 0.04 | 146% | |
| 29 | OTC 3 | Second | A4715 | 70.3 | 79.9 ± 65.1 | 0.12 | 486% | 365 ± 59 |
| | (16139) | prep | A4716 | 46.3 | | 0.06 | 239% | |
| | | | A4717 | 28.7 | | 0.07 | 270% | |
| | | | A4720 | 174.1 | | 0.11 | 465% | |
| 30 | OTC 6 | Second | A4719 | 92.5 | 143.3 ± 57.1 | 0.30 | | 802 ± 79 |
| | (17115) | prep | A4721 | 218.4 | | 0.23 | 934% | |
| | | | A4718 | 156.1 | | 0.15 | 624% | |
| | | | A4708 | 106.0 | | 0.21 | 847% | |

TABLE 29
Viral Genome Copy Number Quantification of FIG. 28.

| Group | Virus name | Virus | Mouse | Viral | Mean ± |
|---|---|---|---|---|---|
| (male) | (code) | batch | code | Genomes/cell | SD |
| 27 | OTCwt | Second | A4723 | 1.07 | 0.5 ± 0.4 |
| | (16137) | Prep | A4724 | 0.30 | |
| | | | A4709 | 0.15 | |
| | | | A4710 | 0.58 | |
| 28 | OTC 1 | Second | A4711 | 0.30 | 1.0 ± 1.6 |
| | (16138) | Prep | A4712 | 0.45 | |
| | | | A4713 | 0.03 | |
| | | | A4722 | 3.39 | |
| 29 | OTC 3 | Second | A4715 | 0.23 | 0.3 ± 0.3 |
| | (16139) | prep | A4716 | 0.26 | |
| | | | A4717 | 0.14 | |
| | | | A4720 | 0.72 | |
| 30 | OTC 6 | Second | A4719 | 0.14 | 0.3 ± 0.2 |
| | (17115) | Prep | A4721 | 0.44 | |
| | | | A4718 | 0.47 | |
| | | | A4708 | 0.21 | |

TABLE 30
Experimental Groups and Doses - OTC^{spf-ash} Males-Intermediate Dose.

| | Virus | | | | Mouse | | | |
|---|---|---|---|---|---|---|---|---|
| Group | name | Virus | Mouse | Vg/ | weight | Vg/ | μl | |
| (male) | (code) | batch | code | Kg | (gr) | mouse | virus | PBS |
| 32 | OTCwt | Second | A3917 | 5.0E+11 | 20 | 1.00E+10 | 0.83 | 49.17 |
| | (16137) | prep | A3918 | 5.0E+11 | 20 | 1.00E+10 | 0.83 | 49.17 |
| | | | A3919 | 5.0E+11 | 22 | 1.10E+10 | 0.92 | 49.08 |
| | | | A3920 | 5.0E+11 | 22 | 1.10E+10 | 0.92 | 49.08 |
| | | | A3921 | 5.0E+11 | 20 | 1.00E+10 | 0.83 | 49.17 |
| 33 | OTC1 | Second | A3922 | 5.0E+11 | 14 | 7.00E+09 | 0.73 | 49.27 |
| | (16138) | prep | A3923 | 5.0E+11 | 19 | 9.50E+09 | 0.99 | 49.01 |
| | | | A3924 | 5.0E+11 | 22 | 1.10E+10 | 1.15 | 48.85 |
| | | | A3925 | 5.0E+11 | 22 | 1.10E+10 | 1.15 | 48.85 |
| | | | A3926 | 5.0E+11 | 22 | 1.10E+10 | 1.15 | 48.85 |
| 34 | OTC 3 | Second | A3927 | 5.0E+11 | 20 | 1.00E+10 | 1.52 | 48.48 |
| | (16139) | prep | A3930 | 5.0E+11 | 20 | 1.00E+10 | 1.52 | 48.48 |
| | | | A3931 | 5.0E+11 | 16 | 8.00E+09 | 1.21 | 48.79 |
| | | | A3932 | 5.0E+11 | 18 | 9.00E+09 | 1.36 | 48.64 |
| | | | A3933 | 5.0E+11 | 19 | 9.50E+09 | 1.44 | 48.56 |

TABLE 31
Experimental Groups and Doses - OTC^{spf-ash} Males-High Dose.

| | Virus | | | | Mouse | | | |
|---|---|---|---|---|---|---|---|---|
| Group | name | Virus | Mouse | Vg/ | weight | | μl | |
| (male) | (code) | batch | code | Kg | (gr) | Vg/mouse | virus | PBS |
| 35 | OTCwt | Second | A3937 | 1.0E+12 | 22 | 2.20E+10 | 1.83 | 48.17 |
| | (16137) | prep | A3938 | 1.0E+12 | 22 | 2.20E+10 | 1.83 | 48.17 |
| | | | A3939 | 1.0E+12 | 22 | 2.20E+10 | 1.83 | 48.17 |
| | | | A3940 | 1.0E+12 | 19 | 1.90E+10 | 1.58 | 48.42 |
| | | | A3941 | 1.0E+12 | 19 | 1.90E+10 | 1.58 | 48.42 |
| 36 | OTC1 | Second | A3976 | 1.0E+12 | 20 | 2.00E+10 | 2.08 | 47.92 |
| | (16138) | prep | A3977 | 1.0E+12 | 23 | 2.30E+10 | 2.40 | 47.60 |
| | | | A3978 | 1.0E+12 | 22 | 2.20E+10 | 2.29 | 47.71 |
| | | | A3979 | 1.0E+12 | 21 | 2.10E+10 | 2.19 | 47.81 |
| | | | A3991 | 1.0E+12 | 18 | 1.80E+10 | 1.88 | 48.13 |
| 37 | OTC 3 | Second | A3992 | 1.0E+12 | 17 | 1.70E+10 | 2.58 | 47.42 |
| | (16139) | prep | A3993 | 1.0E+12 | 15 | 1.50E+10 | 2.27 | 47.73 |
| | | | A3994 | 1.0E+12 | 21 | 2.10E+10 | 3.18 | 46.82 |
| | | | A3995 | 1.0E+12 | 21 | 2.10E+10 | 3.18 | 46.82 |
| | | | A3996 | 1.0E+12 | 19 | 1.90E+10 | 2.88 | 47.12 |
| 38 | No | | A3934 | | | | | |
| | treatment | | A3935 | | | | | |

TABLE 32
Experimental Groups and Doses - OTC^{spf-ash} Females-Intermediate Dose.

| | Virus | | | | Mouse | | | |
|---|---|---|---|---|---|---|---|---|
| Group | name | Virus | Mouse | Vg/ | weight | | μl | |
| female | (code) | batch | code | Kg | (gr) | Vg/mouse | virus | PBS |
| 32 | OTCwt | Second | A3865 | 5.0E+11 | 20.8 | 1.04E+10 | 0.87 | 49.13 |
| | (16137) | prep | A3866 | 5.0E+11 | 20.4 | 1.02E+10 | 0.85 | 49.15 |
| | | | A3867 | 5.0E+11 | 25 | 1.25E+10 | 1.04 | 48.96 |
| | | | A3868 | 5.0E+11 | 21.5 | 1.08E+10 | 0.90 | 49.10 |
| | | | A3869 | 5.0E+11 | 23 | 1.15E+10 | 0.96 | 49.04 |
| 33 | OTC1 | Second | A3870 | 5.0E+11 | 22 | 1.10E+10 | 1.15 | 48.85 |
| | (16138) | prep | A3871 | 5.0E+11 | 21 | 1.05E+10 | 1.09 | 48.91 |
| | | | A3872a | 5.0E+11 | 23 | 1.15E+10 | 1.20 | 48.80 |
| | | | A3872b | 5.0E+11 | 20.5 | 1.03E+10 | 1.07 | 48.93 |
| | | | A3873 | 5.0E+11 | 26.5 | 1.33E+10 | 1.38 | 48.62 |
| 34 | OTC 3 | Second | A3874 | 5.0E+11 | 23.3 | 1.17E+10 | 1.77 | 48.23 |
| | (16139) | prep | A3875 | 5.0E+11 | 25 | 1.25E+10 | 1.89 | 48.11 |
| | | | A3876 | 5.0E+11 | 23.5 | 1.18E+10 | 1.78 | 48.22 |
| | | | A3877 | 5.0E+11 | 22 | 1.10E+10 | 1.67 | 48.33 |
| | | | A3878 | 5.0E+11 | 21 | 1.05E+10 | 1.59 | 48.41 |

TABLE 33
Experimental Groups and Doses - OTC^{spf-ash} Females-High Dose.

| | Virus | | | | Mouse | | | |
|---|---|---|---|---|---|---|---|---|
| Group | name | Virus | Mouse | Vg/ | weight | Vg/ | μl | |
| female | (code) | batch | code | Kg | (gr) | mouse | virus | PBS |
| 35 | OTCwt | Second | A3888 | 1.0E+12 | 23 | 2.30E+10 | 1.92 | 48.08 |
| | (16137) | prep | A3889 | 1.0E+12 | 22 | 2.20E+10 | 1.83 | 48.17 |
| | | | A3890 | 1.0E+12 | 21 | 2.10E+10 | 1.75 | 48.25 |
| | | | A3891 | 1.0E+12 | 22 | 2.20E+10 | 1.83 | 48.17 |
| | | | A3892 | 1.0E+12 | 20 | 2.00E+10 | 1.67 | 48.33 |
| 36 | OTC1 | Second | A3893 | 1.0E+12 | 22 | 2.20E+10 | 2.29 | 47.71 |
| | (16138) | prep | A3894 | 1.0E+12 | 21.5 | 2.15E+10 | 2.24 | 47.76 |
| | | | A3895 | 1.0E+12 | 18.5 | 1.85E+10 | 1.93 | 48.07 |
| | | | A3896 | 1.0E+12 | 18 | 1.80E+10 | 1.88 | 48.13 |
| | | | A3897 | 1.0E+12 | 18.5 | 1.85E+10 | 1.93 | 48.07 |
| 37 | OTC 3 | Second | A3898 | 1.0E+12 | 18 | 1.80E+10 | 2.73 | 47.27 |
| | (16139) | prep | A3899 | 1.0E+12 | 19 | 1.90E+10 | 2.88 | 47.12 |
| | | | A3900 | 1.0E+12 | 17 | 1.70E+10 | 2.58 | 47.42 |
| | | | A3901 | 1.0E+12 | 17 | 1.70E+10 | 2.58 | 47.42 |
| | | | A3902 | 1.0E+12 | 19 | 1.90E+10 | 2.88 | 47.12 |

TABLE 34
Western Blot Quantification of FIG. 29.

| | | | | Band | | |
|---|---|---|---|---|---|---|
| | | | | intensity | | |
| | | | | OTC/ | Fold | |
| | Virus | | | Tubulin | change | |
| Group | name | Virus | Mouse | (normalized | OTC- | Mean ± |
| (male) | (code) | batch | code | on Vg) | wt | SD |
| 32 | OTCwt | Second | A3917 | 4.26 | 84% | 100 ± 25 |
| | (16137) | prep | A3918 | 5.54 | 109% | |
| | | | A3919 | 3.28 | 64% | |
| | | | A3920 | 6.43 | 126% | |
| | | | A3921 | 5.97 | 117% | |
| 33 | OTC1 | Second | A3922 | 6.26 | 123% | 214 ± 108 |
| | (16138) | prep | A3923 | 10.38 | 204% | |
| | | | A3924 | 5.06 | 99% | |
| | | | A3925 | 17.94 | 352% | |
| | | | A3926 | 14.82 | 291% | |
| 32 | OTCwt | Second | A3917 | 7.04 | 102% | 100 ± 27 |
| | (16137) | prep | A3918 | 3.95 | 57% | |
| | | | A3919 | 7.17 | 104% | |
| | | | A3920 | 9.11 | 132% | |
| | | | A3921 | 7.18 | 104% | |
| 34 | OTC 3 | Second | A3927 | 15.66 | 227% | 171 ± 70 |
| | (16139) | prep | A3930 | 5.40 | 78% | |
| | | | A3931 | 10.25 | 149% | |
| | | | A3932 | 15.78 | 229% | |
| | | | A3933 | | | |

TABLE 35
OTC Catalytic Activity Quantification of FIG. 29.

| | Virus | | | | OD | Fold | |
|---|---|---|---|---|---|---|---|
| Group | name | Virus | Mouse | mmol | 490nm/ | change/ | Mean ± |
| (male) | (code) | batch | code | citrullline | vg | wtOTC | SD |
| 32 | OTCwt | Second | A3917 | 6.06 | 6.39 | 56% | 100 ± 32.7 |
| | (16137) | prep | A3918 | 6.35 | 9.16 | 80% | |
| | | | A3919 | 7.02 | 14.63 | 128% | |
| | | | A3920 | 6.30 | 11.69 | 102% | |
| | | | A3921 | 5.77 | 15.34 | 134% | |
| 33 | OTC1 | Second | A3922 | 20.20 | 50.28 | 439% | 194 ± 143.3 |
| | (16138) | prep | A3923 | 8.80 | 21.59 | 189% | |
| | | | A3924 | 6.83 | 13.17 | 115% | |
| | | | A3925 | 8.66 | 8.97 | 78% | |
| | | | A3926 | 8.85 | 16.76 | 147% | |
| 34 | OTC 3 | Second | A3927 | 9.33 | 20.85 | 182% | 397 ± 275.5 |
| | (16139) | prep | A3930 | 41.18 | 90.58 | 792% | |
| | | | A3931 | 21.93 | 43.28 | 378% | |
| | | | A3932 | 19.05 | 27.16 | 237% | |
| | | | A3933 | | | | |
| 38 | Untreated | | A3934 | 5.14 | | | |

TABLE 36
Viral Genome Copy Number Quantification of FIG. 29.

| | Virus | | | Viral | |
|---|---|---|---|---|---|
| | name | Virus | Mouse | Genomes/ | Mean ± |
| Group (male) | (code) | batch | code | cell | SD |
| 32 | OTCwt | Second | A3917 | 0.05 | 0.086 ± 0.034 |
| | (16137) | prep | A3918 | 0.06 | |
| | | | A3919 | 0.13 | |
| | | | A3920 | 0.08 | |
| | | | A3921 | 0.11 | |
| 33 | OTC1 | Second | A3922 | 0.31 | 0.146 ± 0.098 |
| | (16138) | prep | A3923 | 0.13 | |
| | | | A3924 | 0.07 | |
| | | | A3925 | 0.07 | |
| | | | A3926 | 0.15 | |
| 34 | OTC 3 | Second | A3927 | 0.15 | 0.325 ± 0.160 |
| | (16139) | prep | A3930 | 0.52 | |
| | | | A3931 | 0.37 | |
| | | | A3932 | 0.26 | |
| | | | A3933 | | |

TABLE 37
Urinary orotic acid measurement in OTC^{spf-ash} male mice in FIG. 30.

| | Virus | | μmol Orotic acid/mmol creatinine | | | | |
|---|---|---|---|---|---|---|---|
| Group | name | Mouse | | T2 | T4 | T6 | T8 |
| (male) | (code) | code | T0 | weeks | weeks | weeks | weeks |
| 32 | OTCwt | A3917 | 377.0 | 605.7 | 480.6 | 324.2 | 387.2 |
| | (16137) | A3918 | 558.2 | 378.2 | 387.5 | 575.0 | 415.9 |
| | | A3919 | 345.4 | 336.2 | 175.8 | 150.0 | 218.5 |
| | | A3920 | 348.2 | 237.6 | 127.0 | 438.8 | 321.0 |
| | | A3921 | 306.8 | 348.0 | 120.9 | 591.4 | 399.2 |
| 33 | OTC1 | A3922 | 279.3 | 493.6 | 138.9 | 468.0 | 149.5 |
| | (16138) | A3923 | 537.5 | 247.6 | 120.3 | 544.7 | 220.5 |
| | | A3924 | 497.1 | 390.3 | 512.8 | 310.5 | 170.8 |
| | | A3925 | | 351.4 | 264.4 | 250.7 | 308.3 |
| | | A3926 | 469.8 | 391.4 | 263.8 | 152.3 | 232.8 |
| 34 | OTC 3 | A3927 | 546.0 | 372.0 | 315.1 | 177.7 | 304.1 |
| | (16139) | A3930 | 404.2 | 80.9 | 63.2 | 39.1 | 68.8 |
| | | A3931 | 331.7 | 451.5 | 150.6 | 184.5 | 204.8 |
| | | A3932 | 291.9 | 229.1 | 143.0 | 242.7 | 171.9 |
| | | A3933 | 286.9 | 79.2 | 125.9 | 107.1 | 158.4 |
| 38 | un- | A3934 | 400.2 | 561.0 | 525.0 | 689.5 | 535.2 |
| | treated | | | | | | |

TABLE 38
Urinary orotic acid quantification in OTC^{spf-ash} mice in FIG. 30.

| | Virus | | | | |
|---|---|---|---|---|---|
| Group | name | Virus | Mouse | μmol | Mean ± |
| (male) | (code) | batch | code | citrullline | SD |
| 32 | OTCwt | Second | A3917 | 6.06 | 6.3 ± 4.6 |
| | (16137) | prep | A3918 | 6.35 | |
| | | | A3919 | 7.02 | |
| | | | A3920 | 6.30 | |
| | | | A3921 | 5.77 | |
| 33 | OTC1 | Second | A3922 | 20.20 | 10.7 ± 5.4 |
| | (16138) | prep | A3923 | 8.80 | |
| | | | A3924 | 6.83 | |
| | | | A3925 | 8.66 | |
| | | | A3926 | 8.85 | |
| 34 | OTC 3 | Second | A3927 | 9.33 | 22.9 ± 13.3 |
| | (16139) | prep | A3930 | 41.18 | |
| | | | A3931 | 21.93 | |
| | | | A3932 | 19.05 | |
| | | | A3933 | | |
| 38 | Untreated | | A3934 | 5.14 | |
| | C57Bl/6WT | | | 47 | 51.5 ± 6.4 |
| | | | | 56 | |

TABLE 39
Western Blot Quantification of FIG. 31.

| | | | | Band | | | |
|---|---|---|---|---|---|---|---|
| | | | | intensity | | | |
| | | | | OTC/HSP70 | Fold | | |
| | Virus | | | (normalized | change | | |
| Group | name | Virus | Mouse | on | OTC- | | Standard |
| (male) | (code) | batch | code | Vg) | wt | Mean | deviation |
| 35 | OTCwt | Second | A3937 | 0.91 | 54% | 100% | 30 |
| | (16137) | prep | A3938 | 2.06 | 122% | | |
| | | | A3939 | 1.51 | 89% | | |
| | | | A3940 | 2.21 | 131% | | |
| | | | A3941 | 1.77 | 105% | | |
| 36 | OTC1 | Second | A3976 | 4.36 | 258% | 340% | 184 |
| | (16138) | prep | A3977 | 9.51 | 562% | | |
| | | | A3978 | 1.27 | 75% | | |
| | | | A3979 | 7.11 | 420% | | |
| | | | A3991 | 6.55 | 387% | | |
| 35 | OTCwt | Second | A3937 | 0.96 | 51% | 100% | 47 |
| | (16137) | prep | A3938 | 1.56 | 84% | | |
| | | | A3939 | 1.52 | 81% | | |
| | | | A3940 | 3.30 | 177% | | |
| | | | A3941 | 2.01 | 107% | | |
| 37 | OTC 3 | Second | A3992 | 25.33 | 1355% | 535% | 483 |
| | (16139) | prep | A3993 | 3.65 | 196% | | |
| | | | A3994 | 10.23 | 547% | | |
| | | | A3995 | 3.44 | 184% | | |
| | | | A3996 | 7.33 | 392% | | |

TABLE 40
OTC Catalytic Activity Quantification of FIG. 31.

| | Virus | | | | OD | Fold | |
|---|---|---|---|---|---|---|---|
| Group | name | Virus | Mouse | μmol | 490nm/ | change/wt | Mean ± |
| (male) | (code) | batch | code | citrullline | vg | OTS | SD |
| 35 | OTCwt | Second | A3937 | 59.7 | 0.18 | 81% | 100 ± 32 |
| | (16137) | prep | A3938 | 35.8 | 0.25 | 115% | |
| | | | A3939 | 32.9 | 0.14 | 64% | |
| | | | A3940 | 12.8 | 0.20 | 93% | |
| | | | A3941 | 44.0 | 0.32 | 147% | |
| 36 | OTC1 | Second | A3976 | 50.6 | 1.12 | 516% | 369 ± 214 |
| | (16138) | prep | A3977 | 16.8 | 0.81 | 372% | |
| | | | A3978 | 41.0 | 0.22 | 101% | |
| | | | A3979 | 15.8 | 0.49 | 224% | |
| | | | A3991 | 51.6 | 1.37 | 632% | |
| 37 | OTC 3 | Second | A3992 | 11.5 | 0.81 | 373% | 419 ± 399 |
| | (16139) | prep | A3993 | 15.1 | 0.46 | 210% | |
| | | | A3994 | 54.4 | 2.43 | 1120% | |
| | | | A3995 | 28.1 | 0.42 | 192% | |
| | | | A3996 | 17.6 | 0.44 | 200% | |
| 38 | Untreated | | | 6.1 | | | |

TABLE 41
Viral Genome Copy Number Quantification of FIG. 31.

| | | | | VIRAL | | | |
|---|---|---|---|---|---|---|---|
| | Virus | | | GENOMES/ | | | Group |
| Group | name | Virus | Mouse | cell | | | Mean ± |
| (male) | (code) | batch | code | Exp 1 | Exp 2 | Mean | SD |
| 35 | OTCwt | Second | A3937 | 13.8 | 3.4 | 8.6 | 4.4 ± 2.8 |
| | (16137) | prep | A3938 | 5.5 | 1.5 | 3.5 | |
| | | | A3939 | 8.7 | 2.5 | 5.6 | |
| | | | A3940 | 1.6 | 0.3 | 1.0 | |
| | | | A3941 | 5.3 | 1.7 | 3.5 | |
| 36 | OTC1 | Second | A3976 | 1.9 | 0.5 | 1.2 | 1.5 ± 1.8 |
| | (16138) | prep | A3977 | 0.6 | 0.2 | 0.4 | |
| | | | A3978 | 7.3 | 2.1 | 4.7 | |
| | | | A3979 | 0.9 | 0.3 | 0.6 | |
| | | | A3991 | 1.4 | 0.4 | 0.9 | |
| 37 | OTC 3 | Second | A3992 | 0.3 | 0.2 | 0.2 | 0.7 ± 0.4 |
| | (16139) | prep | A3993 | 0.9 | 0.2 | 0.6 | |
| | | | A3994 | 1.0 | 0.3 | 0.7 | |
| | | | A3995 | 2.3 | 0.6 | 1.4 | |
| | | | A3996 | 1.2 | 0.4 | 0.8 | |

TABLE 42
OTC Catalytic Activity Quantification of FIG. 32.

| | Virus | | | | OD | Fold | |
|---|---|---|---|---|---|---|---|
| Group | name | Virus | Mouse | μmol | 490nm/ | change/ | |
| female | (code) | batch | code | citrullline | vg | wtOTC | Mean ± SD |
| 32 | OTCwt | Second | A3865 | 59.7 | 0.11 | 34% | 100 ± 82 |
| | (16137) | prep | A3866 | 35.8 | 0.26 | 81% | |
| | | | A3867 | 32.9 | 0.60 | 192% | |
| | | | A3868 | 12.8 | 0.56 | 180% | |
| | | | A3869 | 44.0 | 0.04 | 13% | |
| 33 | OTC1 | Second | A3870 | 50.6 | 0.72 | 230% | 101 ± 73 |
| | (16138) | prep | A3871 | 16.8 | 0.27 | 86% | |
| | | | A3872a | 41.0 | 0.21 | 66% | |
| | | | A3872b | 15.8 | 0.17 | 54% | |
| | | | A3873 | 51.6 | 0.22 | 69% | |
| 34 | OTC 3 | Second | A3874 | 11.5 | 0.13 | 43% | 61 ± 27 |
| | (16139) | prep | A3875 | 15.1 | 0.15 | 48% | |
| | | | A3876 | 54.4 | 0.13 | 41% | |
| | | | A3877 | 28.1 | 0.33 | 104% | |
| | | | A3878 | 17.6 | 0.22 | 70% | |
| | Untreated | | | 6.1 | | | |

TABLE 43
Viral Genome Quantification of FIG. 32.

| | Virus | | | | |
|---|---|---|---|---|---|
| Group | name | Virus | Mouse | Viral | |
| (female) | (code) | batch | code | Genomes/cell | Mean ± SD |
| 32 | OTCwt | Second | A3865 | 1.13 | 1.77 ± 1.70 |
| | (16137) | prep | A3866 | 1.62 | |
| | | | A3867 | 0.59 | |
| | | | A3868 | 0.78 | |
| | | | A3869 | 4.74 | |
| 33 | OTC1 | Second | A3870 | 0.99 | 2.33 ± 0.96 |
| | (16138) | prep | A3871 | 2.84 | |
| | | | A3872a | 2.35 | |
| | | | A3872b | 1.93 | |
| | | | A3873 | 3.55 | |
| 34 | OTC 3 | Second | A3874 | 7.39 | 3.60 ± 2.33 |
| | (16139) | prep | A3875 | 2.18 | |
| | | | A3876 | 3.48 | |
| | | | A3877 | 1.31 | |
| | | | A3878 | 3.60 | |

TABLE 44
Western Blot Quantification of FIG. 33.

| | | | | Band | | | |
|---|---|---|---|---|---|---|---|
| | | | | intensity | Fold | | |
| | Virus | | | OTC/HSP70 | change | | |
| Group | name | Virus | Mouse | (normalized | OTC- | | Standard |
| (female) | (code) | batch | code | on Vg) | wt | Mean | deviation |
| 35 | OTCwt | Second | A3888 | 0.458 | 97% | 100% | 30 |
| | (16137) | prep | A3889 | 0.563 | 119% | | |
| | | | A3890 | 0.230 | 49% | | |
| | | | A3891 | 0.537 | 114% | | |
| | | | A3892 | 0.574 | 122% | | |
| 36 | OTC1 | Second | A3893 | 0.642 | 136% | 237% | 130 |
| | (16138) | prep | A3894 | 1.534 | 325% | | |
| | | | A3895 | 0.447 | 95% | | |
| | | | A3896 | 1.932 | 409% | | |
| | | | A3897 | 1.044 | 221% | | |
| 35 | OTCwt | Second | A3888 | 0.458 | 93% | 100% | 33 |
| | (16137) | prep | A3889 | 0.659 | 134% | | |
| | | | A3890 | 0.230 | 47% | | |
| | | | A3891 | 0.537 | 109% | | |
| | | | A3892 | 0.574 | 117% | | |
| 37 | OTC 3 | Second | A3898 | 1.483 | 302% | 351% | 88 |
| | (16139) | prep | A3899 | 1.365 | 278% | | |
| | | | A3900 | 2.228 | 453% | | |
| | | | A3901 | 2.166 | 441% | | |
| | | | A3902 | 1.378 | 280% | | |

TABLE 45
OTC Catalytic Activity Quantification of FIG. 33.

| | Virus | | | | OD | Fold | |
|---|---|---|---|---|---|---|---|
| Group | name | Virus | Mouse | μmol | 490 | change/ | |
| (female) | (code) | batch | code | citrullline | nm/vg | wtOTC | Mean ± SD |
| 35 | OTCwt | Second | A3888 | 90.12 | 0.254 | 102% | 100 ± 19 |
| | (16137) | prep | A3889 | 51.61 | 0.287 | 116% | |
| | | | A3890 | 58.07 | 0.223 | 90% | |
| | | | A3891 | 37.20 | 0.178 | 72% | |
| | | | A3892 | 69.72 | 0.298 | 120% | |
| 36 | OTC1 | Second | A3893 | 88.54 | 0.334 | 135% | 218 ± 136 |
| | (16138) | prep | A3894 | 84.37 | 0.889 | 358% | |
| | | | A3895 | 92.24 | 0.176 | 71% | |
| | | | A3896 | 59.49 | 0.914 | 368% | |
| | | | A3897 | 57.83 | 0.389 | 157% | |
| 37 | OTC 3 | Second | A3898 | 37.05 | 0.375 | 151% | 265 ± 226 |
| | (16139) | prep | A3899 | 77.13 | 0.458 | 185% | |
| | | | A3900 | 53.98 | 0.606 | 245% | |
| | | | A3901 | 52.95 | 1.627 | 656% | |
| | | | A3902 | 35.94 | 0.213 | 86% | |
| 38 | untreated | | | 28.70 | | | |

TABLE 46
Viral Genome Quantification of FIG. 33.

| | Virus | | | | |
|---|---|---|---|---|---|
| Group | name | Virus | Mouse | Viral | |
| (female) | (code) | batch | code | Genomes/cell | Mean ± SD |
| 35 | OTCwt | Second | A3888 | 3.35 | 1.96 ± 0.91 |
| | (16137) | prep | A3889 | 1.26 | |
| | | | A3890 | 1.99 | |
| | | | A3891 | 1.00 | |
| | | | A3892 | 1.98 | |
| 36 | OTC1 | Second | A3893 | 2.49 | 1.99 ± 1.83 |
| | (16138) | prep | A3894 | 0.87 | |
| | | | A3895 | 4.98 | |
| | | | A3896 | 0.50 | |
| | | | A3897 | 1.13 | |
| 37 | OTC 3 | Second | A3898 | 0.47 | 0.72 ± 0.48 |
| | (16139) | prep | A3899 | 1.50 | |
| | | | A3900 | 0.64 | |
| | | | A3901 | 0.23 | |
| | | | A3902 | 0.76 | |

TABLE 47
Experimental Conditions and Doses CO21-High dose.

| | Virus | | | | Mouse | | | |
|---|---|---|---|---|---|---|---|---|
| Group | name | Virus | Mouse | | weight | | μl | |
| (male) | (code) | batch | code | Vg/Kg | (gr) | Vg/mouse | virus | PBS |
| 39 | OTCwt | Second | A4670 | 1.0E+12 | 22 | 2.20E+10 | 1.83 | 48.17 |
| | (16137) | prep | A4672 | 1.0E+12 | 25 | 2.50E+10 | 2.08 | 47.92 |
| | | | A4673 | 1.0E+12 | 22 | 2.20E+10 | 1.83 | 48.17 |
| | | | A4681 | 1.0E+12 | 30 | 3.00E+10 | 2.50 | 47.50 |
| | | | A4692 | 1.0E+12 | 30 | 3.00E+10 | 2.50 | 47.50 |
| 40 | OTC 3 | Second | A4696 | 1.0E+12 | 20 | 2.00E+10 | 2.08 | 47.92 |
| | (16139) | prep | A4675 | 1.0E+12 | 23 | 2.30E+10 | 2.40 | 47.60 |
| | | | A4676 | 1.0E+12 | 26 | 2.60E+10 | 2.71 | 47.29 |
| | | | A4677 | 1.0E+12 | 21 | 2.10E+10 | 2.19 | 47.81 |
| | | | A4678 | 1.0E+12 | 24 | 2.40E+10 | 2.50 | 47.50 |
| 41 | OTC 21 | Third | A4876 | 1.0E+12 | 17 | 1.70E+10 | 2.58 | 47.42 |
| | (17115) | prep | A4877 | 1.0E+12 | 25 | 2.50E+10 | 3.79 | 46.21 |
| | | | A4878 | 1.0E+12 | 21 | 2.10E+10 | 3.18 | 46.82 |
| | | | A4882 | 1.0E+12 | 25 | 2.50E+10 | 3.79 | 46.21 |
| | | | A4884 | 1.0E+12 | 24 | 2.40E+10 | 3.64 | 46.36 |

TABLE 48
Experimental Conditions and Doses CO21-Intermediate dose.

| | Virus | | | | Mouse | | | |
|---|---|---|---|---|---|---|---|---|
| Group | name | Virus | Mouse | | weight | | μl | |
| (male) | (code) | batch | code | Vg/Kg | (gr) | Vg/mouse | virus | PBS |
| 42 | OTCwt | Second | A5395 | 5.0E+11 | 24 | 1.20E+10 | 0.9 | 49.1 |
| | (16137) | prep | A5398 | 5.0E+11 | 25.5 | 1.28E+10 | 0.9 | 49.1 |
| | | | A5399 | 5.0E+11 | 29 | 1.45E+10 | 1.1 | 48.9 |
| | | | A5400 | 5.0E+11 | 28 | 1.40E+10 | 1.0 | 49.0 |
| | | | A5393 | 5.0E+11 | 23 | 1.15E+10 | 0.9 | 49.1 |
| 43 | OTC 21 | Third | A5394 | 5.0E+11 | 23 | 1.15E+10 | 6.8 | 43.2 |
| | (17115) | prep | A5396 | 5.0E+11 | 24 | 1.20E+10 | 7.1 | 42.9 |
| | | | A5401 | 5.0E+11 | 31 | 1.55E+10 | 9.1 | 40.9 |
| | | | A5402 | 5.0E+11 | 26 | 1.30E+10 | 7.6 | 42.4 |
| | | | A5409 | 5.0E+11 | 29 | 1.45E+10 | 8.5 | 41.5 |

TABLE 49
Experimental Conditions and Doses CO21-Low dose.

| | Virus | | | | Mouse | | | |
|---|---|---|---|---|---|---|---|---|
| Group | name | Virus | Mouse | | weight | | μl | |
| (male) | (code) | batch | code | Vg/Kg | (gr) | Vg/mouse | virus | PBS |
| 44 | OTCwt | Second | A5272 | 2.5E+11 | 38 | 9.50E+09 | 0.7 | 49.3 |
| | (16137) | prep | A5273 | 2.5E+11 | 25 | 6.25E+09 | 0.5 | 49.5 |
| | | | A5278 | 2.5E+11 | 26 | 6.50E+09 | 0.5 | 49.5 |
| | | | A5281 | 2.5E+11 | 23 | 5.75E+09 | 0.4 | 49.6 |
| | | | A5272 | 2.5E+11 | 38 | 9.50E+09 | 0.7 | 49.3 |
| 45 | OTC 21 | Third | A5275 | 2.5E+11 | 23 | 5.75E+09 | 3.4 | 46.6 |
| | (17115) | prep | A5277 | 2.5E+11 | 22.5 | 5.63E+09 | 3.3 | 46.7 |
| | | | A5279 | 2.5E+11 | 26 | 6.50E+09 | 3.8 | 46.2 |
| | | | A5284 | 2.5E+11 | 30 | 7.50E+09 | 4.4 | 45.6 |
| | | | A5361 | 2.5E+11 | 24 | 6.00E+09 | 3.5 | 46.5 |
| 46 | untreated | | A5410 | | | | | |
| | | | A4795 | | | | | |
| | | | A4701 | | | | | |

TABLE 50
Western Blot Quantification of FIG. 34.

| | | | | Band | | |
|---|---|---|---|---|---|---|
| | | | | intensity | | |
| | | | | OTC/ | | |
| | | | | HSP70 | | |
| | Virus | | | (nor- | Fold | |
| Group | name | Virus | Mouse | malized | change | |
| (male) | (code) | batch | code | on Vg) | OTC-wt | Mean ± SD |
| 39 | OTCwt | Second | A4670 | 0.036 | 112% | 100 ± 49 |
| | (16137) | prep | A4672 | 0.022 | 69% | |
| | | | A4673 | 0.022 | 69% | |
| | | | A4681 | 0.020 | 62% | |
| | | | A4692 | 0.062 | 193% | |
| 40 | OTC 3 | Second | A4696 | 0.030 | 94% | 417 ± 120 |
| | (16139) | prep | A4675 | 0.151 | 470% | |
| | | | A4676 | 0.172 | 537% | |
| | | | A4677 | 0.064 | 199% | |
| | | | A4678 | 0.145 | 451% | |
| 39 | OTCwt | Second | A4670 | 0.051 | 195% | 100 ± 54 |
| | (16137) | prep | A4672 | 0.017 | 64% | |
| | | | A4673 | 0.013 | 50% | |
| | | | A4681 | 0.020 | 77% | |
| | | | A4692 | 0.035 | 132% | |
| 41 | OTC 21 | Third | A4876 | 0.105 | 404% | 406 ± .54 |
| | (17115) | prep | A4877 | 0.089 | 341% | |
| | | | A4878 | 0.091 | 348% | |
| | | | A4882 | 0.083 | 317% | |
| | | | A4884 | 0.119 | 457% | |

TABLE 51
OTC Catalytic Activity Quantification of FIG. 34.

| | Virus | | | | | | |
|---|---|---|---|---|---|---|---|
| Group | name | Virus | Mouse | μmol | OD 490 | Fold change/ | |
| (male) | (code) | batch | code | citrullline | nm/vg | wtOTC | Mean ± SD |
| 39 | OTCwt | Second | A4670 | 66.4 | 0.016 | 106% | 100 ± 68 |
| | (16137) | prep | A4672 | 6.4 | 0.010 | 64% | |
| | | | A4673 | 1.5 | 0.007 | 43% | |
| | | | A4681 | 3.5 | 0.010 | 63% | |
| | | | A4692 | 20.1 | 0.035 | 223% | |
| 40 | OTC 3 | Second | A4696 | 12.1 | 0.008 | 54% | 179 ± 81 |
| | (16139) | prep | A4675 | 58.9 | 0.047 | 301% | |
| | | | A4676 | 51.5 | 0.032 | 206% | |
| | | | A4677 | 90.6 | 0.021 | 137% | |
| | | | A4678 | 49.6 | 0.031 | 197% | |
| 41 | OTC 21 | Third | A4876 | 20.8 | 0.048 | 306% | 265 ± 85 |
| | (17115) | prep | A4877 | 47.0 | 0.044 | 284% | |
| | | | A4878 | 20.4 | 0.017 | 112% | |
| | | | A4882 | 113.6 | 0.052 | 350% | |
| | | | A4884 | 117.1 | 0.042 | 271% | |
| | untreated | | | 6.1 | | | |

TABLE 52
Viral Genome Quantification of FIG. 34.

| | Virus | | | | |
|---|---|---|---|---|---|
| Group | name | Virus | Mouse | Viral | |
| (male) | (code) | batch | code | Genomes/cell | Mean ± SD |
| 39 | OTCwt | Second | A4670 | 39.6 | 14.66 ± 14.25 |
| | (16137) | prep | A4672 | 10.7 | |
| | | | A4673 | 10.9 | |
| | | | A4681 | 8.5 | |
| | | | A4692 | 3.6 | |
| 40 | OTC 3 | Second | A4696 | 16.5 | 16.74 ± 9.66 |
| | (16139) | prep | A4675 | 10.5 | |
| | | | A4676 | 10.1 | |
| | | | A4677 | 33.4 | |
| | | | A4678 | 13.2 | |
| 41 | OTC 21 | Third prep | A4876 | 6.4 | 14.38 ± 7.34 |
| | (17115) | | A4877 | 11.3 | |
| | | | A4878 | 11.3 | |
| | | | A4882 | 17.4 | |
| | | | A4884 | 25.5 | |

TABLE 53
Urinary Orotic Acid Quantification of FIG. 35.

| | Virus | | μmol Orotic acid/mmol creatinine | | | | |
|---|---|---|---|---|---|---|---|
| Group | name | Mouse | | T2 | T3 | T6 | T8 |
| (male) | (code) | code | T0 | weeks | weeks | weeks | weeks |
| 39 | OTCwt | A4670 | 235.4 | 29.4 | 43.1 | 17.4 | 31.6 |
| | (16137) | A4672 | 1648.9 | 79.0 | 33.9 | 47.4 | 29.3 |
| | | A4673 | 127.2 | 90.0 | 17.8 | 62.8 | 55.4 |
| | | A4681 | 75.1 | 64.1 | 70.4 | 92.2 | 114.1 |
| | | A4692 | 197.3 | 38.3 | 19.5 | 43.1 | 43.0 |
| 40 | OTC 3 | A4696 | 307.62 | 49.2 | 39.8 | 101.4 | 85.14 |
| | (16139) | A4675 | 214.95 | 79.3 | 51.3 | 200.5 | 113.52 |
| | | A4676 | 575.46 | 154.3 | 60 | 62.3 | 38.29 |
| | | A4677 | 252.58 | 61 | 61.1 | 74.2 | 65.9 |
| | | A4678 | 512.53 | 66.1 | 70 | 91.6 | 44.73 |
| 41 | OTC 21 | A4876 | 316.0 | 28.3 | 14.8 | 31.3 | 32.2 |
| | (17115) | A4877 | 217.2 | 37.1 | 21.5 | 30.2 | 24.7 |
| | | A4878 | 273.6 | 6.3 | 34.5 | 63.2 | 49.9 |
| | | A4882 | 157.1 | 27.9 | 28.5 | 27.0 | 34.1 |
| | | A4884 | | 31.2 | 14.8 | 31.7 | 38.7 |
| 46 | untreated | | 851.3 | 511.8 | | 309 | 405.2 |

TABLE 54
Urinary Orotic Acid Quantification of FIG. 37.

| | Virus | | μmol Orotic acid/mmol creatinine | | | | |
|---|---|---|---|---|---|---|---|
| Group | name | Mouse | | T2 | T4 | T6 | T8 |
| (male) | (code) | code | T0 | weeks | weeks | weeks | weeks |
| 42 | OTCwt | A5395 | 559.02 | 470.19 | 580.13 | 305.10 | 62.97 |
| | (16137) | A5398 | 429.38 | 78.11 | 31.35 | 68.17 | 116.55 |
| | | A5399 | 519.97 | 46.97 | 50.99 | 56.61 | 165.75 |
| | | A5400 | 440.58 | 64.12 | 167.41 | 224.87 | |
| | | A5393 | 187.13 | 395.56 | 264.16 | 300.05 | |
| 43 | OTC 21 | A5394 | 333.56 | 58.71 | 65.14 | 58.52 | 76.58 |
| | (17115) | A5396 | 559.02 | 23.52 | 31.11 | 23.52 | 56.60 |
| | | A5401 | 429.38 | 44.84 | 53.38 | 92.60 | 75.40 |
| | | A5402 | 519.97 | 20.46 | 23.67 | 35.93 | 58.22 |
| | | A5409 | 405.13 | 55.9 | 20.68 | 72.33 | 95.12 |

TABLE 55
Western Blot Quantification of FIG. 38.

| | | | | Band | | |
|---|---|---|---|---|---|---|
| | | | | intensity | | |
| | | | | OTC/ | | |
| | | | | HSP70 | | |
| | Virus | | | (nor- | Fold | |
| Group | name | Virus | Mouse | malized | change | |
| (male) | (code) | batch | code | on Vg) | OTC-wt | Mean ± SD |
| 42 | OTCwt | Second | A5395 | 0.33 | 51% | 100 ± 49 |
| | (16137) | prep | A5398 | 0.31 | 104% | |
| | | | A5399 | 0.30 | 79% | |
| | | | A5400 | 0.45 | 166% | |
| | | | A5393 | 0.33 | 51% | |
| 43 | OTC 21 | Third | A5394 | 2.01 | 151% | 325 ± 232 |
| | (17115) | prep | A5396 | 1.74 | 165% | |
| | | | A5401 | 1.69 | 352% | |
| | | | A5402 | 0.98 | 715% | |
| | | | A5409 | 1.40 | 242% | |

TABLE 56
Orotic Acid Catalytic Quantification of FIG. 38.

| | Virus | | | | OD | Fold | |
|---|---|---|---|---|---|---|---|
| Group | name | Virus | Mouse | μmol | 490 | change/ | |
| (male) | (code) | batch | code | citrullline | nm/vg | wtOTC | Mean ± SD |
| 42 | OTCwt | Second | A5395 | 1.17 | 0.50 | 209% | 100 ± 74 |
| | (16137) | prep | A5398 | 10.23 | 0.19 | 81% | |
| | | | A5399 | 6.70 | 0.14 | 58% | |
| | | | A5400 | 8.47 | 0.12 | 52% | |
| | | | A5393 | 2.81 | | | |
| 43 | OTC 21 | Third | A5394 | 140.02 | 0.26 | 110% | 220 ± 106 |
| | (17115) | prep | A5396 | 140.48 | 0.77 | 323% | |
| | | | A5401 | 135.60 | 0.55 | 231% | |
| | | | A5402 | 86.99 | 0.77 | 323% | |
| | | | A5409 | 44.61 | 0.27 | 112% | |
| | untreated | | | 1.75 | | | |

TABLE 57
Viral Genome Quantification of FIG. 38.

| | Virus | | | Viral | |
|---|---|---|---|---|---|
| | name | Virus | Mouse | Genomes/ | |
| Group (male) | (code) | batch | code | cell | Mean ± SD |
| 42 | OTCwt | Second | A5395 | 0.17 | 0.74 ± 0.61 |
| | (16137) | prep | A5398 | 1.00 | |
| | | | A5399 | 1.10 | |
| | | | A5400 | 1.40 | |
| | | | A5393 | 0.01 | |
| 43 | OTC | Third | A5394 | 6.77 | 3.19 ± 2.08 |
| | 21 | prep | A5396 | 2.31 | |
| | (17115) | | A5401 | 3.12 | |
| | | | A5402 | 1.47 | |
| | | | A5409 | 2.30 | |

TABLE 58
Western Blot Quantification of FIG. 39.

| | | | | Band | | |
|---|---|---|---|---|---|---|
| | | | | intensity | | |
| | Virus | | | OTC/HSP70 | Fold | |
| Group | name | Virus | Mouse | (normalized | change | Mean ± |
| (male) | (code) | batch | code | on Vg) | OTC-wt | SD |
| 44 | OTCwt | Second | A5272 | 0.90 | 97% | 100 ± 21 |
| | (16137) | prep | A5273 | 0.68 | 74% | |
| | | | A5278 | 0.94 | 102% | |
| | | | A5281 | 1.16 | 126% | |
| 45 | OTC | Third | A5275 | 2.53 | 193% | 192 ± 58 |
| | 21 | prep | A5277 | 1.40 | 275% | |
| | (17115) | | A5279 | 1.16 | 152% | |
| | | | A5284 | 1.98 | 125% | |
| | | | A5361 | 0.90 | 215% | |

TABLE 59
OTC Catalytic Activity Quantification of FIG. 39.

| | Virus | | | | OD | Fold | |
|---|---|---|---|---|---|---|---|
| Group | name | Virus | Mouse | μmol | 490 | change/ | |
| (male) | (code) | batch | code | citrullline | nm/vg | wtOTC | Mean ± SD |
| 44 | OTCwt | Second | A5272 | 11.09 | 0.50 | 153% | 100 ± 38 |
| | (16137) | prep | A5273 | 8.95 | 0.19 | 103% | |
| | | | A5278 | 49.80 | 0.14 | 72% | |
| | | | A5281 | 45.25 | 0.12 | 73% | |
| 45 | OTC 21 | Third | A5275 | 26.30 | 0.26 | 97% | 227 ± 156 |
| | (17115) | prep | A5277 | 2.05 | 0.77 | 489% | |
| | | | A5279 | 10.80 | 0.55 | 229% | |
| | | | A5284 | 164.15 | 0.77 | 197% | |
| | | | A5361 | 15.05 | 0.27 | 121% | |
| | untreated | | | 1.75 | | | |

TABLE 60
Viral Genome Quantification of FIG. 39.

| | Virus | | | Viral | | | |
|---|---|---|---|---|---|---|---|
| Group | name | Virus | Mouse | Genomes/cell | | | |
| (male) | (code) | batch | code | Exp1 | Exp 2 | Mean | Group Mean ± SD |
| 44 | OTCwt | Second | A5272 | 0.31 | 0.5 | 0.41 | 2.19 ± 1.85 |
| | (16137) | prep | A5273 | 0.58 | 1.0 | 0.79 | |
| | | | A5278 | 2.04 | 6.0 | 4.02 | |
| | | | A5281 | 1.65 | 5.4 | 3.54 | |
| 45 | OTC 21 | Third | A5275 | 0.85 | 2.5 | 1.67 | 1.53 ± 1.76 |
| | (17115) | prep | A5277 | 0.13 | 0.1 | 0.11 | |
| | | | A5279 | 0.34 | 0.5 | 0.43 | |
| | | | A5284 | 2.16 | 6.8 | 4.50 | |
| | | | A5361 | 0.62 | 1.3 | 0.94 | |

TABLE 61
Urinary Orotic Acid Quantification of FIG. 40.

| | Virus | | | | | | |
|---|---|---|---|---|---|---|---|
| Group | name | Mouse | μmol Orotic acid/mmol creatinine | | | | |
| (male) | (code) | code | T0 | T2 weeks | T4 weeks | T6 weeks | T8 weeks |
| 44 | OTCwt | A5272 | 204.8 | 89.63 | 78.47 | 130.8 | 111.84 |
| | (16137) | A5273 | 1723.4 | 278.25 | 408.82 | 150.96 | 118 |
| | | A5278 | 139.9 | 339.79 | 932.68 | 283 | 86.2 |
| | | A5281 | 335.8 | 36.62 | 51.76 | 73.16 | 87 |
| 45 | OTC 21 | A5277 | 400 | 378.36 | 225.73 | 177.03 | 261.88 |
| | (17115) | A5279 | 355 | 59.82 | 67.54 | 33.74 | 67.47 |
| | | A5284 | 600 | 55.58 | 82.89 | 88.18 | 114.99 |
| | | A5361 | 1255.9 | 92.19 | 180.56 | 140.2 | 107.25 |
| | untreated | | 1140 | 334 | 279.9 | 511.8 | 252.4 |

TABLE 62
OTC Catalytic Activity Quantification in FIG. 41.

| Group (male) | Virus name (code) | Dose | Mouse code | μmol citrullline | Mean ± SD |
|---|---|---|---|---|---|
| 44 | OTCwt (16137) | 2.5E11 | A5272 | 1.26 | 6.02 ± 5.7 |
| | | | A5273 | 11.50 | |
| | | | A5278 | 10.33 | |
| | | | A5281 | 1.00 | |
| 42 | OTCwt (16137) | 5.0E11 | A5395 | 5.59 | 3.94 ± 1.65 |
| | | | A5398 | 5.11 | |
| | | | A5399 | 3.35 | |
| | | | A5400 | 4.23 | |
| | | | A5393 | 1.41 | |
| 39 | OTCwt (16137) | 1.0E12 | A4670 | 66.4 | 20.28 ± 38 |
| | | | A4672 | 6.4 | |
| | | | A4673 | 5.0 | |
| | | | A4681 | 3.5 | |
| | | | A4692 | 20.1 | |
| 41 | OTC 21 (17115) | 2.5E11 | A5275 | 5.6 | 12.8 ± 18.2 |
| | | | A5277 | 1.8 | |
| | | | A5279 | 40 | |
| | | | A5284 | 3.8 | |
| 43 | OTC 21 (17115) | 5E11 | A5394 | 50 | 48.11 ± 24.66 |
| | | | A5396 | 70.24 | |
| | | | A5401 | 67.8 | |
| | | | A5402 | 43.5 | |
| | | | A5409 | 9 | |
| 45 | OTC 21 (17115) | 1E12 | A4876 | 47 | 49.72 ± 39.1 |
| | | | A4877 | 20 | |
| | | | A4878 | 113.6 | |
| | | | A4882 | 52 | |
| | | | A4884 | 16 | |
| | untreated | | | 0.87 | 1.04 ± 0.23 |
| | | | | 1.20 | |
| | C57-WT | | | 49 | 45.63 ± 4.9 |
| | | | | 39.9 | |
| | | | | 48 | |

TABLE 63
Ammonia Challenge Experimantal Groups and Dosage.

| | Virus | | | | Mouse | | | |
|---|---|---|---|---|---|---|---|---|
| Group | name | Virus | Mouse | | weight | | μl | |
| (male) | (code) | batch | code | Vg/Kg | (gr) | Vg/mouse | virus | PBS |
| 47 | OTCwt | Second | A5819 | 5.0E+11 | 22 | 2.20E+10 | 1.83 | 48.17 |
| | (16137) | prep | A5820 | 5.0E+11 | 25 | 2.50E+10 | 2.08 | 47.92 |
| | | | A5830 | 5.0E+11 | 22 | 2.20E+10 | 1.83 | 48.17 |
| | | | A5935 | 5.0E+11 | 30 | 3.00E+10 | 2.50 | 47.50 |
| 48 | OTC 21 | Third | A5821 | 5.0E+11 | 20 | 2.00E+10 | 2.08 | 47.92 |
| | (17115) | prep | A5822 | 5.0E+11 | 23 | 2.30E+10 | 2.40 | 47.60 |
| | | | A5932 | 5.0E+11 | 26 | 2.60E+10 | 2.71 | 47.29 |
| | | | A5934 | 5.0E+11 | 21 | 2.10E+10 | 2.19 | 47.81 |
| 49 | untreated | | A6416 | | 17 | | | 50 |
| | | | A6417 | | 25 | | | 50 |
| | | | A6422 | | 21 | | | 50 |
| | | | A6423 | | 25 | | | 50 |
| 50 | Wt mice | | A5405 | | 26 | | | 50 |
| | | | A5406 | | 30 | | | 50 |
| | | | A5404 | | 21 | | | 50 |
| | | | A5965 | | 30 | | | 50 |

TABLE 65
First Ammonia Challenge Quantification of FIG. 42.

| | | | | μmol | | | | |
|---|---|---|---|---|---|---|---|---|
| | Virus | | | OA/ | Behavioral test (score) | | | |
| | name | Mouse | mmol | mmol | | Sound | | |
| Group | (code) | code | NH₄ | creatinine | Seizures | sensitivity | Gait | Total |
| 47 | OTCwt | A5819 | 2.58 | 65.3 | 1.5 | 1 | 2 | 4.5 |
| | (16137) | A5820 | 1.27 | 125.2 | 2 | 3 | 2 | 7 |
| | | A5830 | 0.92 | 93.0 | 1.5 | 2 | 2 | 5.5 |
| | | A5935 | 2.60 | 49.3 | 2 | 2 | 2 | 6 |
| 48 | OTC | A5821 | 1.19 | 45.6 | 2 | 3 | 2 | 7 |
| | 21 | A5822 | 0.94 | 50.0 | 2 | 3 | 2 | 7 |
| | (17115) | A5932 | 1.32 | 57.2 | 2 | 3 | 2 | 7 |
| | | A5934 | 0.96 | 45.6 | 2 | 2.5 | 2 | 6.5 |
| 49 | untreated | A6416 | 2.62 | 210 | 0 | 1 | 0 | 1 |
| | | A6417 | 4.59 | 353 | 1 | 1 | 1 | 3 |
| | | A6422 | 5.96 | 420 | 0 | 1 | 1 | 2 |
| | | A6423 | 10.48 | | 0 | 0 | 0 | 0 |
| 50 | Wt | A5405 | 3.86 | 70 | 1 | 2 | 2 | 5 |
| | mice | A5406 | 3.62 | 38 | 2 | 2 | 2 | 6 |
| | | A5404 | 3.24 | | 2 | 2 | 2 | 6 |
| | | A5965 | 3.24 | | 2 | 2 | 2 | 6 |

TABLE 66
Second Ammonia Challenge Quantification of FIG. 44.

| | | | | μmol | | | | |
|---|---|---|---|---|---|---|---|---|
| | Virus | | | OA/ | Behavioral test (score) | | | |
| | name | Mouse | mmol | mmol | | Sound | | |
| Group | (code) | code | NH₄ | creatinine | Seizures | sensitivity | Gait | Total |
| 47 | OTCwt | A5819 | 3.37 | 140.7 | 1 | 1 | 2 | 4 |
| | (16137) | A5820 | 2.56 | 117.7 | 2 | 2 | 1 | 5 |
| | | A5830 | 2.84 | 93.9 | 1.5 | 2 | 1 | 4.5 |
| | | A5935 | 1.58 | 89.2 | 1 | 2 | 2 | 5 |
| 48 | OTC 21 | A5821 | 1.86 | 90.9 | 2 | 2 | 2 | 6 |
| | (17115) | A5822 | 3.62 | 78.8 | 2 | 2 | 1 | 5 |
| | | A5932 | 1.66 | 81.8 | 1 | 2 | 1 | 4 |
| | | A5934 | 2.44 | 81.2 | 1 | 2 | 2 | 5 |
| 49 | untreated | A6416 | 5.57 | 582.7 | 0 | 0 | 0 DIED | 0 |
| | | A6417 | 4.23 | 341.1 | 0 | 0 | 0 DIED | 0 |
| | | A6422 | 4.76 | 472.0 | 0 | 1 | 1 | 2 |
| | | A6423 | 6.80 | 378.0 | 0 | 1 | 1 | 0 |
| 50 | Wt mice | A5405 | 3.87 | 102.3 | 1 | 2 | 2 | 5 |
| | | A5406 | 2.54 | 108.0 | 2 | 2 | 2 | 6 |
| | | A5404 | 2.62 | | 2 | 2 | 2 | 6 |

TABLE 67
Western Blot Quantification of FIG. 44.

| | | | | Band | | |
|---|---|---|---|---|---|---|
| | | | | intensity | | |
| | Virus | | | OTC/Tubulin | Fold | |
| Group | name | Mouse | | (normalized | change | Mean ± |
| (male) | (code) | code | Vg/Kg | on Vg) | OTC-wt | SD |
| 47 | OTCwt | A5819 | 5.00E+11 | 1.179 | 138% | 100 ± 29 |
| | (16137) | A5820 | 5.00E+11 | 0.741 | 87% | |
| | | A5830 | 5.00E+11 | 0.601 | 70% | |
| | | A5935 | 5.00E+11 | 0.905 | 106% | |
| 48 | OTC 21 | A5821 | 5.00E+11 | 6.145 | 717% | 600.7 ± 115 |
| | (17115) | A5822 | 5.00E+11 | 4.436 | 518% | |
| | | A5932 | 5.00E+11 | 5.826 | 680% | |
| | | A5934 | 5.00E+11 | 4.183 | 488% | |

TABLE 68
OTC Catalytic Activity Quantification of FIG. 44.

| | Virus | | | |
|---|---|---|---|---|
| Group | name | Mouse | μmol | Mean ± |
| (male) | (code) | code | citrullline | SD |
| 47 | OTCwt | A5819 | 7.27 | 6.75 ± 2.9 |
| | (16137) | A5820 | 9.67 | |
| | | A5830 | 2.77 | |
| | | A5935 | 7.27 | |
| 48 | OTC 21 | A5821 | 78.57 | 95.10 ± 22.15 |
| | (17115) | A5822 | 76.37 | |
| | | A5932 | 102.07 | |
| | | A5934 | 123.37 | |
| 49 | untreated | A6416 | 1.87 | 0.87 ± 1.4 |
| | | A6417 | −0.13 | |
| | | A6422 | | |
| | | A6423 | | |
| 50 | Wt mice | A5405 | 92.67 | 82.52 ± 14.3 |
| | | A5406 | 72.37 | |
| | | A5404 | | |
| | | A5965 | | |

TABLE 69
Viral Genome Quantification of FIG. 44.

| | Virus | | Viral | |
|---|---|---|---|---|
| Group | name | Mouse | Genomes/ | |
| (male) | (code) | code | cell | Mean ± SD |
| 47 | OTCwt | A5819 | 1.4 | 1.0 ± 0.5 |
| | (16137) | A5820 | 1.0 | |
| | | A5830 | 0.3 | |
| | | A5935 | 1.3 | |
| 48 | OTC 21 | A5821 | 1.0 | 1.5 ± 0.5 |
| | (17115) | A5822 | 1.5 | |
| | | A5932 | 1.3 | |
| | | A5934 | 2.1 | |

1. A method comprising:
concomitantly administering an adeno-associated virus (AAV) vector to a subject that has or is suspected of having a urea cycle disorder and synthetic nanocarriers coupled to an immunosuppressant, wherein the AAV vector comprises a nucleic acid sequence that encodes an enzyme associated with the urea cycle disorder and an expression control sequence.

2. The method of claim 1, wherein the urea cycle disorder is ornithine transcarbamylase synthetase (OTC) deficiency.

3. The method of claim 1, wherein the AAV vector and synthetic nanocarriers coupled to an immunosuppressant are in an amount effective to reduce humoral and cellular immune responses to the AAV vector.

4. The method of claim 1, wherein the AAV vector and synthetic nanocarriers coupled to an immunosuppressant are concomitantly administered during early disease onset.

5. The method of claim 1, wherein the subject is not administered a steroid as an additional therapeutic.

6. The method of claim 1, wherein the method further comprises administering a steroid as an additional therapeutic in a reduced amount.

7. The method of claim 1, wherein the subject has been previously administered the AAV vector and synthetic nanocarriers coupled to an immunosuppressant concomitantly.

8. The method of claim 1, wherein the method further comprises administering the AAV vector to the subject at a subsequent point in time.

9. The method of claim 1, wherein the concomitant administration of the AAV vector and synthetic nanocarriers coupled to an immunosuppressant is repeated.

10. The method of claim 1, wherein the sequence encoding the enzyme associated with the urea cycle disorder is a codon-optimized sequence.

11. The method of claim 10, wherein the enzyme associated with the urea cycle disorder is OTC.

12. The method of claim 11, wherein the sequence encoding the OTC is a sequence that encodes the OTC of OTC-CO3 or OTC-CO21.

13. The method of claim 11, wherein the sequence encoding the OTC is a sequence as set forth in SEQ ID NO: 1-11 or 13, or a portion thereof.

14. The method of claim 11, wherein the sequence encoding the OTC is a sequence that encodes the OTC of SEQ ID NO: 13.

15-42. (canceled)

43. A composition comprising:
a dose of the AAV vector of claim 1.

44. The composition of claim 43, wherein the composition further comprises a dose of synthetic nanocarriers of claim 1.

45. The composition of claim 43, wherein the composition is a kit.

46-47. (canceled)

48. A composition comprising a nucleic acid comprising the nucleic acid sequence of claim 1.

49-54. (canceled)

55. A composition comprising a nucleic acid comprising a sequence as set forth in any one of the sequences provided herein, such as SEQ ID NO: 4, 8 or 9, or a portion thereof, and that encodes an OTC.

56-58. (canceled)

59. A composition comprising a viral vector comprising a composition of claim 48.

60-62. (canceled)
